(19) United States
(12) Patent Application Publication
(10) Pub. No.: US 2022/0119786 A1
(43) Pub. Date: Apr. 21, 2022

(54) GENETICALLY-MODIFIED CELLS COMPRISING A MODIFIED TRANSFERRIN GENE

(71) Applicant: Precision BioSciences, Inc., Durham, NC (US)

(72) Inventors: Cassandra Gorsuch, Durham, NC (US); Derek Jantz, Durham, NC (US); James Jefferson Smith, Morrisville, NC (US); Clayton Beard, Durham, NC (US)

(73) Assignee: Precision BioSciences, Inc., Durham, NC (US)

(21) Appl. No.: 17/422,118

(22) PCT Filed: Jan. 10, 2020
(86) PCT No.: PCT/US20/13198

§ 371 Date: Jul. 9, 2021

Related U.S. Application Data
(60) Provisional application No. 62/790,848, filed on Jan. 10, 2019.

(51) Int. Cl. C12N 9/22; C12N 15/86; C07K 14/79; A61K 38/46

(52) U.S. Cl. CPC C12N 9/22; A61K 38/465; C07K 14/79; C12N 15/86

(57) ABSTRACT

Disclosed herein are engineered nucleases that bind and cleave a recognition sequence within intron 1 of a transferrin gene, and methods of using such engineered nucleases to produce a genetically-modified eukaryotic cell comprising a modified transferrin gene. Further provided are pharmaceutical compositions and methods for treatment of a variety of conditions through expression of a polypeptide of interest encoded by an exogenous nucleic acid molecule inserted in intron 1 of a transferrin gene and expressed under the control of the endogenous transferrin promoter.

### FIELD OF THE INVENTION

The invention relates to the field of molecular biology and recombinant nucleic acid technology. In particular, the invention relates to engineered nucleases having specificity for a recognition sequence within a transferrin gene. The invention further relates to the use of such nucleases in the preparation of genetically-modified eukaryotic cells comprising a transferrin gene that has been modified within intron 1 for the purpose of expressing a polypeptide of interest.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jan. 10, 2020, is named P1090.70031WO00-SEQ, and is 60,000 bytes in size.

### BACKGROUND OF THE INVENTION

Gene therapy remains a logical approach for the treatment of diseases resulting from an insufficiency of natural protein production, or for diseases that could benefit from therapeutic protein expression. Traditional gene therapy approaches rely on the use of an exogenous promoter to drive expression of a therapeutic transgene. Although such transgenes are not intended to integrate into a subject's genome, a low level of random integration does occur. This integration is not precise, and there is a risk that the exogenous promoter could land next to, and change the expression of, other genes in the subject's chromosome (e.g., oncogenes), thereby raising the risk of cancer and other genotoxicities.

One way to overcome these challenges involves the use of a nuclease-based, targeted integration approach in which a nuclease is used to generate a cleavage site at a specific locus of a gene, and the coding sequence for a therapeutic transgene is inserted into the cleavage site in such a manner that its expression is driven by the gene's endogenous promoter.

The present invention focuses on the use of the transferrin gene for this “promoter stealing” approach. Transferrin is a highly-expressed secreted glycoprotein, which functions to transport iron from the liver and intestine to proliferating cells in the body. The transferrin polypeptide is encoded by the transferrin (“TF”) gene. The secretion of transferrin from cells is enabled by a signal peptide that is fused to the protein during translation. The N-terminal fragment of the signal peptide is encoded by exon 1 of the transferrin gene, and the remaining C-terminal fragment is encoded by the first 14 base pairs of exon 2.

The present invention relies on the insertion of an exogenous nucleic acid molecule into intron 1 of the transferrin gene between exon 1 and exon 2. In general, this exogenous nucleic acid molecule comprises a coding sequence for a polypeptide of interest along with several other elements, including an exogenous splice acceptor sequence, which allows expression of the construct to be driven by the endogenous transferrin promoter. The exogenous nucleic acid molecule also comprises bases encoding the C-terminal fragment of a signal peptide, such as the transferrin signal peptide, allowing for production of a full-length signal peptide for protein secretion. A polyA signal is included at the 3′ end of the exogenous nucleic acid molecule to terminate translation and prevent expression of the endogenous transferrin protein itself.

To facilitate the insertion of an exogenous nucleic acid molecule into intron 1 of the transferrin gene, the present invention utilizes engineered, site-specific, rare-cutting nucleases. Methods for producing engineered, site-specific nucleases are known in the art. For example, zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein that binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). A CRISPR endonuclease comprises two components: (1) clustered regularly interspaced short palindromic repeats-associated endonuclease; and (2) a short “guide RNA” comprising a ˜20 nucleotide targeting sequence that directs the nuclease to a location of interest in the genome. By expressing multiple guide RNAs in the same cell, each having a different targeting sequence, it is possible to target DNA breaks simultaneously to multiple sites in in the genome.

In the preferred embodiment of the invention, the DNA break-inducing agent is an engineered homing endonuclease (also called a “meganuclease”). Homing endonucleases are a group of naturally-occurring nucleases, which recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Homing endonucleases are commonly grouped into four families: the LAGLIDADG (SEQ ID NO: 2) family, the GIY-YIG family, the His-Cys box family, and the HNH family. These families are characterized by structural motifs, which affect catalytic activity and recognition sequence. For instance, members of the LAGLIDADG (SEQ ID NO: 2) family are characterized by having either one or two copies of the conserved LAGLIDADG (SEQ ID NO: 2) motif (see Chevalier et al. (2001), Nucleic Acids Res. 29(18): 3757-3774). The LAGLIDADG (SEQ ID NO: 2) homing endonucleases with a single copy of the LAGLIDADG (SEQ ID NO: 2) motif form homodimers, whereas members with two copies of the LAGLIDADG (SEQ ID NO: 2) motif are found as monomers.

I-CreI (SEQ ID NO: 1) is a member of the LAGLIDADG (SEQ ID NO: 2) family of homing endonucleases, which recognizes and cuts a 22 basepair recognition sequence in the chloroplast chromosome of the algae Chlamydomonas reinhardtii. Genetic selection techniques have been used to modify the wild-type I-CreI cleavage site preference (Sussman et al. (2004), J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58). Methods for rationally-designing mono-LAGLIDADG (SEQ ID NO: 2) homing endonucleases were previously described, which are capable of comprehensively redesigning I-CreI and other homing endonucleases to target widely-divergent DNA sites, including sites in mammalian, yeast, plant, bacterial, and viral genomes (WO 2007/047859).

As first described in WO 2009/059195, I-CreI and its engineered derivatives are normally dimeric but can be fused into a single polypeptide using a short peptide linker that joins the C-terminus of a first subunit to the N-terminus of a second subunit (Li, et al. (2009) Nucleic Acids Res. 37:1650-62; Grizot, et al. (2009) Nucleic Acids Res. 37:5405-19.) Thus, a functional “single-chain” meganuclease can be expressed from a single transcript. These engineered meganucleases demonstrate extremely low frequency of off-target cutting.

In the present invention, the particular architecture of the transferrin gene has been used in combination with site-specific engineered nucleases to improve on previous promoter stealing approaches in several respects. Because exon 1 of the transferrin gene only encodes a fragment of the signal peptide, and no part of the transferrin protein itself, a therapeutic protein can be produced that does not include any fragments of the endogenous transferrin protein. The present invention also avoids the expression of fragmented endogenous proteins and provides for the insertion of a simpler construct compared to those used in previous methods in other genes. Further, the present invention allows for the administration of just two deliverables: the first providing a nuclease, and the second providing a repair template. In some cases, for example, both may be delivered by an AAV. In other cases, the nuclease may be delivered as an mRNA encapsulated in a lipid nanoparticle, and the repair template delivered by AAV. Previous methods described in the art require at least three deliverables to provide all nuclease components and a repair template to target cells. Accordingly, the present invention fulfills a need in the art for improved gene editing approaches to enable expression of exogenous polypeptides of interest in vivo.

### SUMMARY OF THE INVENTION

The present invention provides engineered nucleases that bind and cleave a recognition sequence within intron 1 of a transferrin gene (e.g., intron 1 of the human transferrin gene (SEQ ID NO: 4) or intron 1 of the mouse transferrin gene (SEQ ID NO: 12)). Further provided are methods comprising the delivery of a template nucleic acid encoding an exogenous nucleic acid molecule (e.g., encoding a polypeptide of interest) and a nucleic acid encoding an engineered nuclease to a eukaryotic cell in order to produce a genetically-modified eukaryotic cell having a modified transferrin gene capable of driving expression of the polypeptide of interest using the endogenous transferrin promoter. Additionally, the present invention includes pharmaceutical compositions and methods for treatment of a variety of conditions through expression of a polypeptide of interest encoded by an exogenous nucleic acid sequence positioned within intron 1 of a transferrin gene at an engineered nuclease cleavage site.

In one aspect, the invention provides an engineered meganuclease that binds and cleaves a recognition sequence within intron 1 of a transferrin gene, wherein the engineered meganuclease comprises a first subunit and a second subunit, wherein the first subunit binds to a first recognition half-site of the recognition sequence and comprises a first hypervariable (HVR1) region, and wherein the second subunit binds to a second recognition half-site of the recognition sequence and comprises a second hypervariable (HVR2) region.

In some embodiments, the recognition sequence comprises SEQ ID NO: 19. In some such embodiments, the HVR1 region comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence corresponding to residues 215-270 of SEQ ID NO: 23. In certain embodiments, the HVR1 region comprises an amino acid sequence corresponding to residues 215-270 of SEQ ID NO: 23 with up to 1, 2, 3, 4, 5, 6, 7, 8, 9, 10, or 11 amino acid substitutions. In some such embodiments, the HVR1 region comprises one or more residues corresponding to residues 215, 217, 219, 221, 223, 224, 229, 231, 233, 235, 237, 259, 261, 266, and 268 of SEQ ID NO: 23. In some such embodiments, the HVR1 region comprises residues corresponding to residues 215, 217, 219, 221, 223, 224, 229, 231, 233, 235, 237, 259, 261, 266, and 268 of SEQ ID NO: 23. In some embodiments, the HVR1 region comprises Y, R, K, or D at a residue corresponding to residue 257 of SEQ ID NO: 23. In specific embodiments, the HVR1 region comprises residues 215-270 of SEQ ID NO: 23.

In particular embodiments, the first subunit comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 198-344 of SEQ ID NO: 23. In some embodiments, the first subunit comprises an amino acid sequence corresponding to residues 198-344 of SEQ ID NO: 23 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In certain embodiments, the first subunit comprises G, S, or A at a residue corresponding to residue 210 of SEQ ID NO: 23. In certain embodiments, the first subunit comprises E, Q, or K at a residue corresponding to residue 271 of SEQ ID NO: 23. In some embodiments, the first subunit comprises a residue corresponding to residue 271 of SEQ ID NO: 23. In some embodiments, the first subunit comprises residues 198-344 of SEQ ID NO: 23.

In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 24-79 of SEQ ID NO: 23. In certain embodiments, the HVR2 region comprises an amino acid sequence corresponding to residues 24-79 of SEQ ID NO: 23 with up to 1, 2, 3, 4, 5, 6, 7, 8, 9, 10, or 11 amino acid substitutions.

In other embodiments, the HVR2 region comprises one or more residues corresponding to residues 24, 26, 28, 30, 32, 33, 38, 40, 42, 44, 46, 68, 70, 75, and 77 of SEQ ID NO: 23. In particular embodiments, the HVR2 region comprises residues corresponding to residues 24, 26, 28, 30, 32, 33, 38, 40, 42, 44, 46, 68, 70, 75, and 77 of SEQ ID NO: 23. In particular embodiments, the HVR2 region comprises a residue corresponding to residue 41 of SEQ ID NO: 23. In some embodiments, the HVR2 region comprises Y, R, K, or D at a residue corresponding to residue 66 of SEQ ID NO: 23. In particular embodiments, the HVR2 region comprises residues 24-79 of SEQ ID NO: 23.

In some embodiments, the second subunit comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 7-153 of SEQ ID NO: 23. In particular embodiments, the second subunit comprises an amino acid sequence corresponding to residues 7-153 of SEQ ID NO: 23 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In certain embodiments, the second subunit comprises G, S, or A at a residue corresponding to residue 19 of SEQ ID NO: 23. In certain embodiments, the second subunit comprises E, Q, or K at a residue corresponding to residue 80 of SEQ ID NO: 23. In some embodiments, the second subunit comprises a residue corresponding to residue 80 of SEQ ID NO: 23. In some embodiments, the second subunit comprises residues 7-153 of any one of SEQ ID NO: 23.

In some embodiments, the first subunit of the engineered meganuclease has at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 198-344 of SEQ ID NO: 23, and the second subunit comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 7-153 of SEQ ID NO: 23. In certain embodiments, the first subunit and/or the second subunit can comprise up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions relative to residues 198-344 and residues 7-153, respectively, of SEQ ID NO: 23.

In some embodiments, the engineered meganuclease comprises a linker, wherein the linker covalently joins the first subunit and the second subunit.

In particular embodiments, the engineered meganuclease comprises the amino acid sequence of SEQ ID NO: 23.

In some embodiments, the recognition sequence comprises SEQ ID NO: 21. In some such embodiments, the HVR1 region comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence corresponding to residues 215-270 of SEQ ID NO: 26. In certain embodiments, the HVR1 region comprises an amino acid sequence corresponding to residues 215-270 of SEQ ID NO: 26 with up to 1, 2, 3, 4, 5, 6, 7, 8, 9, 10, or 11 amino acid substitutions. In some such embodiments, the HVR1 region comprises one or more residues corresponding to residues 215, 217, 219, 221, 223, 224, 229, 231, 233, 235, 237, 259, 261, 266, and 268 of SEQ ID NO: 26. In some such embodiments, the HVR1 region comprises residues corresponding to residues 215, 217, 219, 221, 223, 224, 229, 231, 233, 235, 237, 259, 261, 266, and 268 of SEQ ID NO: 26. In some embodiments, the HVR1 region comprises Y, R, K, or D at a residue corresponding to residue 257 of SEQ ID NO: 26. In particular embodiments, the HVR1 region comprises residues 215-270 of SEQ ID NO: 26.

In particular embodiments, the first subunit comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 198-344 of SEQ ID NO: 26. In some embodiments, the first subunit comprises an amino acid sequence corresponding to residues 198-344 of SEQ ID NO: 26 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In certain embodiments, the first subunit comprises G, S, or A at a residue corresponding to residue 210 of SEQ ID NO: 26. In certain embodiments, the first subunit comprises E, Q, or K at a residue corresponding to residue 271 of SEQ ID NO: 26. In some embodiments, the first subunit comprises a residue corresponding to residue 271 of SEQ ID NO: 26. In some embodiments, the first subunit comprises residues 198-344 of SEQ ID NO: 26.

In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 24-79 of SEQ ID NO: 26. In certain embodiments, the HVR2 region comprises an amino acid sequence corresponding to residues 24-79 of SEQ ID NO: 26 with up to 1, 2, 3, 4, 5, 6, 7, 8, 9, 10, or 11 amino acid substitutions.

In particular embodiments, the HVR2 region comprises one or more residues corresponding to residues 24, 26, 28, 30, 32, 33, 38, 40, 42, 44, 46, 68, 70, 75, and 77 of SEQ ID NO: 26. In some particular embodiments, the HVR2 region comprises residues corresponding to residues 24, 26, 28, 30, 32, 33, 38, 40, 42, 44, 46, 68, 70, 75, and 77 of SEQ ID NO: 26. In some embodiments, the HVR2 region comprises Y, R, K, or D at a residue corresponding to residue 66 of SEQ ID NO: 26. In some embodiments, the HVR2 region comprises residues 24-79 of SEQ ID NO: 26.

In some embodiments, the second subunit comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 7-153 of SEQ ID NO: 26. In particular embodiments, the second subunit comprises an amino acid sequence corresponding to residues 7-153 of SEQ ID NO: 26 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In certain embodiments, the second subunit comprises G, S, or A at a residue corresponding to residue 19 of SEQ ID NO: 26. In certain embodiments, the second subunit comprises E, Q, or K at a residue corresponding to residue 80 of SEQ ID NO: 26. In some embodiments, the second subunit comprises a residue corresponding to residue 80 of SEQ ID NO: 26. In some embodiments, the second subunit comprises residues 7-153 of any one of SEQ ID NO: 26.

In some embodiments, the first subunit of the engineered meganuclease has at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 198-344 of SEQ ID NO: 26, and the second subunit comprises an amino acid sequence having at least 80%, at least 85%, at least 90%, at least 91%, at least 92%, at least 93%, at least 94%, at least 95%, at least 96%, at least 97%, at least 98%, at least 99% or 100%, sequence identity to an amino acid sequence corresponding to residues 7-153 of SEQ ID NO: 26. In certain embodiments, the first subunit and/or the second subunit can comprise up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions relative to residues 198-344 and residues 7-153, respectively, of SEQ ID NO: 26.

In some embodiments, the engineered meganuclease comprises a linker, wherein the linker covalently joins the first subunit and the second subunit.

In particular embodiments, the engineered meganuclease comprises the amino acid sequence of SEQ ID NO: 26.

In some aspects, the invention provides a nucleic acid sequence encoding any engineered meganuclease of the invention. In a particular embodiment, the polynucleotide is an mRNA. In one such embodiment, the mRNA is packaged within a lipid nanoparticle.

In one embodiment, the invention provides a recombinant DNA construct comprising a nucleic acid sequence encoding any engineered meganuclease of the invention. In one such embodiment, the recombinant DNA construct encodes a viral vector comprising the nucleic acid sequence encoding the engineered meganuclease. In such an embodiment, the viral vector is an adenoviral vector, a lentiviral vector, a retroviral vector, or an adeno-associated viral (AAV) vector. In a particular embodiment, the viral vector is a recombinant AAV vector. In one embodiment, the invention provides a viral vector comprising a nucleic acid sequence encoding any engineered meganuclease disclosed herein. In some embodiments, the viral vector is an adenoviral vector, a lentiviral vector, a retroviral vector, or an adeno-associated viral (AAV) vector. In a particular embodiment, the viral vector is a recombinant AAV vector. In some embodiments, the recombinant AAV vector comprises a polynucleotide comprising, from 5′ to 3′, a first inverted terminal repeat (ITR), a nucleic acid sequence encoding any engineered meganuclease disclosed herein, and a second ITR. In particular embodiments, the recombinant AAV vector comprises a polynucleotide comprising, from 5′ to 3′, a first ITR, a promoter, a nucleic acid sequence encoding any engineered meganuclease disclosed herein, a polyA signal, and a second ITR., wherein the promoter is operably linked to (i.e., drives expression of) the engineered meganuclease.

In another aspect, the invention provides a method for producing a genetically-modified eukaryotic cell comprising an exogenous nucleic acid molecule encoding a polypeptide of interest inserted into a chromosome of the eukaryotic cell, the method comprising introducing into a eukaryotic cell one or more nucleic acids including: (a) a nucleic acid encoding any engineered meganuclease of the invention, wherein the engineered meganuclease is expressed in the eukaryotic cell; and (b) a template nucleic acid comprising the exogenous nucleic acid molecule; wherein the engineered meganuclease produces a cleavage site in the chromosome at a recognition sequence comprising SEQ ID NO: 19 or 21; and wherein the exogenous nucleic acid molecule is inserted into the chromosome at the cleavage site. In some embodiments, the exogenous nucleic acid molecule further comprises sequences homologous to sequences flanking the cleavage site and the exogenous nucleic acid molecule is inserted at the cleavage site by homologous recombination.

In one embodiment of the method, the eukaryotic cell is a mammalian cell. In a particular embodiment, the mammalian cell is selected from a human cell, non-human primate cell, or a mouse cell. For example, the mammalian cell is a mammalian hepatocyte. In certain embodiments, the hepatocyte is within the liver of a human, a non-human primate, or a mouse.

In a particular embodiment of the method, the nucleic acid encoding the engineered meganuclease is introduced into the eukaryotic cell by an mRNA or a viral vector. In one such embodiment, the mRNA is packaged within a lipid nanoparticle. In another such an embodiment, the viral vector is an adenoviral vector, a lentiviral vector, a retroviral vector, or an adeno-associated viral (AAV) vector. In a particular embodiment, the viral vector is a recombinant AAV vector. In some embodiments, the recombinant AAV vector comprises a polynucleotide comprising, from 5′ to 3′, a first inverted terminal repeat (ITR), a nucleic acid sequence encoding any engineered meganuclease disclosed herein, and a second ITR. In particular embodiments, the recombinant AAV vector comprises a polynucleotide comprising, from 5′ to 3′, a first ITR, a promoter, a nucleic acid sequence encoding any engineered meganuclease disclosed herein, a polyA signal, and a second ITR., wherein the promoter is operably linked to (i.e., drives expression of) the engineered meganuclease.

In some embodiments, the template nucleic acid is introduced into the eukaryotic cell by a viral vector. In some such embodiments, the viral vector is an adenoviral vector, a lentiviral vector, a retroviral vector, or an adeno-associated viral (AAV) vector. In a particular embodiment, the viral vector is a recombinant AAV vector. In some embodiments, the recombinant AAV vector comprises, from 5′ to 3′, a first inverted terminal repeat (ITR), the template nucleic acid, and a second ITR.

In another aspect, the invention provides a method for producing a genetically-modified eukaryotic cell comprising an exogenous nucleic acid molecule encoding a polypeptide of interest inserted into a chromosome of the eukaryotic cell, the method comprising: (a) introducing any engineered meganuclease of the invention into a eukaryotic cell; and (b) introducing a template nucleic acid comprising the exogenous nucleic acid molecule into the eukaryotic cell; wherein the engineered meganuclease produces a cleavage site in the chromosome at a recognition sequence comprising SEQ ID NO: 19 or 21; and wherein the exogenous nucleic acid molecule is inserted into the chromosome at the cleavage site. In one embodiment of the method, the exogenous nucleic acid molecule further comprises sequences homologous to sequences flanking the cleavage site and the exogenous nucleic acid molecule is inserted at the cleavage site by homologous recombination.

In some embodiments of the method, the eukaryotic cell is a mammalian cell. In particular embodiments, the mammalian cell is selected from a human cell, non-human primate cell, or a mouse cell. For example, the mammalian cell is a mammalian hepatocyte. In some embodiments, the hepatocyte is within the liver of a human, a non-human primate, or a mouse.

In some embodiments of the method, the template nucleic acid is introduced into the eukaryotic cell by a viral vector. In some such embodiments, the viral vector is an adenoviral vector, a lentiviral vector, a retroviral vector, or an adeno-associated viral (AAV) vector. In a particular embodiment, the viral vector is a recombinant AAV vector. In some embodiments, the recombinant AAV vector comprises a polynucleotide comprising, from 5′ to 3′, a first inverted terminal repeat (ITR), the template nucleic acid, and a second ITR.

In a particular aspect, the invention provides a genetically-modified eukaryotic cell prepared by any methods for producing a genetically-modified eukaryotic cell of the invention disclosed herein.

In another aspect, the invention provides a nucleic acid molecule comprising, from 5′ to 3′: (a) an exogenous splice acceptor sequence; (b) a first nucleic acid sequence encoding a C-terminal fragment of a signal peptide; (c) a second nucleic acid sequence encoding an exogenous polypeptide of interest; and (d) a polyA signal.

In one embodiment, the first nucleic acid sequence is capable of being joined directly to the 3′ end of SEQ ID NO: 8 to generate a coding sequence for a human transferrin signal peptide having at least 80%, at least 85%, at least 90%, at least 95%, or more, sequence identity to SEQ ID NO: 7. In particular embodiments, the encoded transferrin signal peptide comprises SEQ ID NO: 7. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 9. In particular embodiments, the first nucleic acid sequence has at least 80%, at least 85%, at least 90%, at least 95%, or more, sequence identity to SEQ ID NO: 9, wherein the first two nucleotides of the first nucleic acid sequence are GG, GT, GA, or GC. In some embodiments, the first nucleic acid sequence comprises SEQ ID NO: 9.

In another embodiment, the first nucleic acid sequence is capable of being joined directly to the 3′ end of SEQ ID NO: 16 to generate a coding sequence for a mouse transferrin signal peptide having at least 80%, at least 85%, at least 90%, at least 95%, or more, sequence identity to SEQ ID NO: 15. In particular embodiments, the encoded transferrin signal peptide comprises SEQ ID NO: 15. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 17. In particular embodiments, the first nucleic acid sequence has at least 80%, at least 85%, at least 90%, at least 95%, or more, sequence identity to SEQ ID NO: 17, wherein the first two nucleotides of the first nucleic acid sequence are GG, GT, GA, or GC. In some embodiments, the first nucleic acid sequence comprises SEQ ID NO: 17.

In one embodiment, the nucleic acid molecule further comprises a 5′ homology arm, which is positioned 5′ upstream of the exogenous splice acceptor sequence, and a 3′ homology arm which is positioned 3′ downstream of the polyA signal, wherein the 5′ homology arm and the 3′ homology arm are homologous to sequences flanking an engineered nuclease cleavage site of interest within intron 1 of a transferrin gene.

In some embodiments, the exogenous splice acceptor sequence comprises an exogenous branch point comprising CCCTCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 10. In particular embodiments, the exogenous splice acceptor sequence comprises SEQ ID NO: 10.

In some embodiments, the exogenous splice acceptor sequence comprises an exogenous branch point comprising TCCCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 18. In particular embodiments, the exogenous splice acceptor sequence comprises SEQ ID NO: 18.

In certain embodiments, the exogenous splice acceptor sequence is not derived from intron 1 of the transferrin gene.

In some embodiments, the nucleic acid molecule comprises, from 5′ to 3′: (a) the exogenous splice acceptor sequence; (b) the first nucleic acid sequence; (c) an IRES sequence (SEQ ID NO: 29), a T2A sequence (SEQ ID NO: 30), a P2A sequence (SEQ ID NO: 31), an E2A sequence (SEQ ID NO: 32), or an F2A sequence (SEQ ID NO: 33); (d) a third nucleic acid sequence encoding a signal peptide; (e) the second nucleic acid sequence; and (f) the polyA signal. In some such embodiments, the signal peptide encoded by the third nucleic acid sequence comprises an amino acid sequence having at least 80%, at least 85%, at least 90%, at least 95%, or more, sequence identity to SEQ ID NO: 7. In particular embodiments, the signal peptide encoded by the third nucleic acid sequence comprises SEQ ID NO: 7. In some such embodiments, the signal peptide encoded by the third nucleic acid sequence comprises an amino acid sequence having at least 80%, at least 85%, at least 90%, at least 95%, or more, sequence identity to SEQ ID NO: 15. In particular embodiments, the signal peptide encoded by the third nucleic acid sequence comprises SEQ ID NO: 15.

In some embodiments, the exogenous polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

In some embodiments, the polyA signal comprises a nucleic acid sequence having at least 80%, at least 85%, at least 90%, at least 95%, or more, sequence identity to SEQ ID NO: 34. In certain embodiments, the polyA signal comprises a nucleic acid sequence of SEQ ID NO: 34.

In another aspect, the invention provides a genetically-modified eukaryotic cell comprising a modified transferrin gene, wherein the modified transferrin gene comprises an exogenous nucleic acid molecule within intron 1, and wherein the exogenous nucleic acid molecule comprises, from 5′ to 3′: (a) an exogenous splice acceptor sequence; (b) a first nucleic acid sequence encoding a C-terminal fragment of a signal peptide; (c) a second nucleic acid sequence encoding a polypeptide of interest; and (d) a polyA signal.

In one embodiment, the first nucleic acid sequence is capable of being joined directly to the 3′ end of SEQ ID NO: 8 to generate a coding sequence for a human transferrin signal peptide having at least 80%, at least 85%, at least 90%, at least 95%, or more, sequence identity to SEQ ID NO: 7. In particular embodiments, the encoded transferrin signal peptide comprises SEQ ID NO: 7. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 9. In particular embodiments, the first nucleic acid sequence has at least 80%, at least 85%, at least 90%, at least 95%, or more, sequence identity to SEQ ID NO: 9, wherein the first two nucleotides of the first nucleic acid sequence are GG, GT, GA, or GC. In some embodiments, the first nucleic acid sequence comprises SEQ ID NO: 9.

In another embodiment, the first nucleic acid sequence is capable of being joined directly to the 3′ end of SEQ ID NO: 16 to generate a coding sequence for a mouse transferrin signal peptide having at least 80%, at least 85%, at least 90%, at least 95%, or more, sequence identity to SEQ ID NO: 15. In particular embodiments, the encoded transferrin signal peptide comprises SEQ ID NO: 15. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 17. In particular embodiments, the first nucleic acid sequence has at least 80%, at least 85%, at least 90%, at least 95%, or more, sequence identity to SEQ ID NO: 17, wherein the first two nucleotides of the first nucleic acid sequence are GG, GT, GA, or GC. In some embodiments, the first nucleic acid sequence comprises SEQ ID NO: 17.

In some embodiments, the exogenous splice acceptor sequence comprises an exogenous branch point comprising CCCTCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 10. In particular embodiments, the exogenous splice acceptor sequence comprises SEQ ID NO: 10.

In some embodiments, the exogenous splice acceptor sequence comprises an exogenous branch point comprising TCCCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 18. In particular embodiments, the exogenous splice acceptor sequence comprises SEQ ID NO: 18.

In certain embodiments, the exogenous splice acceptor sequence is not derived from intron 1 of the transferrin gene.

In some embodiments, the exogenous nucleic acid molecule comprises, from 5′ to 3′: (a) the exogenous splice acceptor sequence; (b) the first nucleic acid sequence; (c) an IRES sequence (SEQ ID NO: 29), a T2A sequence (SEQ ID NO: 30), a P2A sequence (SEQ ID NO: 31), an E2A sequence (SEQ ID NO: 32), or an F2A sequence (SEQ ID NO: 33); (d) a third nucleic acid sequence encoding a signal peptide; (e) the second nucleic acid sequence; and (f) the polyA signal. In some such embodiments, the signal peptide encoded by the third nucleic acid sequence comprises an amino acid sequence having at least 80%, at least 85%, at least 90%, at least 95%, or more, sequence identity to SEQ ID NO: 7. In particular embodiments, the signal peptide encoded by the third nucleic acid sequence comprises SEQ ID NO: 7. In some such embodiments, the signal peptide encoded by the third nucleic acid sequence comprises an amino acid sequence having at least 80%, at least 85%, at least 90%, at least 95%, or more, sequence identity to SEQ ID NO: 15. In particular embodiments, the signal peptide encoded by the third nucleic acid sequence comprises SEQ ID NO: 15.

In some embodiments, the polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

In some embodiments, the polyA signal comprises a nucleic acid sequence having at least 80%, at least 85%, at least 90%, at least 95%, or more, sequence identity to SEQ ID NO: 34. In particular embodiments, the polyA signal comprises a nucleic acid sequence of SEQ ID NO: 34.

In some embodiments, an endogenous promoter of the modified transferrin gene is operably linked to the exogenous nucleic acid molecule. In such embodiments, the endogenous promoter of the transferrin gene drives expression of the exogenous nucleic acid molecule.

In some embodiments, the genetically-modified eukaryotic cell expresses a polypeptide comprising: (a) a transferrin signal peptide having at least 80%, at least 85%, at least 90%, at least 95%, or more, sequence identity to SEQ ID NO: 7; and (b) the polypeptide of interest; wherein the polypeptide of interest is secreted by the genetically-modified eukaryotic cell. In some embodiments, the genetically-modified eukaryotic cell expresses a polypeptide comprising: (a) a transferrin signal peptide having at least 80%, at least 85%, at least 90%, at least 95%, or more, sequence identity to SEQ ID NO: 15; and (b) the polypeptide of interest; wherein the polypeptide of interest is secreted by the genetically-modified eukaryotic cell.

In some embodiments, the exogenous nucleic acid molecule is positioned within intron 1 at an engineered nuclease cleavage site. In some embodiments, the engineered nuclease cleavage site is within an engineered meganuclease recognition sequence, a TALEN recognition sequence, a compact TALEN recognition sequence, a megaTAL recognition sequence, a zinc finger nuclease recognition sequence, or a CRISPR system nuclease recognition sequence.

In some embodiments, the engineered nuclease cleavage site is within an engineered meganuclease recognition sequence. In certain embodiments, the engineered meganuclease recognition sequence comprises SEQ ID NO: 19 or 21.

In certain embodiments, the engineered nuclease cleavage site is a TALEN cleavage site within a TALEN spacer sequence. In some embodiments, the engineered nuclease cleavage site is a zinc finger nuclease cleavage site within a zinc finger nuclease spacer sequence. In some embodiments, the engineered nuclease cleavage site is within a CRISPR system nuclease recognition sequence.

In some embodiments, the eukaryotic cell is a mammalian cell. In particular embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In another aspect, the invention provides a pharmaceutical composition comprising a pharmaceutically-acceptable carrier and a therapeutically effective amount of: (a) (i) a nucleic acid encoding an engineered nuclease having specificity for a recognition sequence within intron 1 of a transferrin gene, or (ii) an engineered nuclease protein having specificity for a recognition sequence within intron 1 of a transferrin gene; and (b) a template nucleic acid comprising an exogenous nucleic acid molecule, wherein the exogenous nucleic acid molecule comprises, from 5′ to 3′: (i) an exogenous splice acceptor sequence; (ii) a first nucleic acid sequence encoding a C-terminal fragment of a signal peptide; (iii) a second nucleic acid sequence encoding a polypeptide of interest; and (iv) a polyA signal.

In one embodiment, the first nucleic acid sequence is capable of being joined directly to the 3′ end of SEQ ID NO: 8 to generate a coding sequence for a human transferrin signal peptide having at least 80%, at least 85%, at least 90%, at least 95%, or more, sequence identity to SEQ ID NO: 7. In particular embodiments, the encoded transferrin signal peptide comprises SEQ ID NO: 7. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 9. In particular embodiments, the first nucleic acid sequence has at least 80%, at least 85%, at least 90%, at least 95%, or more, sequence identity to SEQ ID NO: 9, wherein the first two nucleotides of the first nucleic acid sequence are GG, GT, GA, or GC. In some embodiments, the first nucleic acid sequence comprises SEQ ID NO: 9.

In another embodiment, the first nucleic acid sequence is capable of being joined directly to the 3′ end of SEQ ID NO: 16 to generate a coding sequence for a mouse transferrin signal peptide having at least 80%, at least 85%, at least 90%, at least 95%, or more, sequence identity to SEQ ID NO: 15. In particular embodiments, the encoded transferrin signal peptide comprises SEQ ID NO: 15. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 17. In particular embodiments, the first nucleic acid sequence has at least 80%, at least 85%, at least 90%, at least 95%, or more, sequence identity to SEQ ID NO: 17, wherein the first two nucleotides of the first nucleic acid sequence are GG, GT, GA, or GC. In some embodiments, the first nucleic acid sequence comprises SEQ ID NO: 17.

In some embodiments, the exogenous nucleic acid molecule further comprises a 5′ homology arm which is positioned 5′ upstream of the exogenous splice acceptor sequence, and a 3′ homology arm which is positioned 3′ downstream of the polyA signal, wherein the 5′ homology arm and the 3′ homology arm are homologous to sequences flanking a cleavage site generated by the engineered nuclease within intron 1 of the transferrin gene.

In some embodiments, the exogenous splice acceptor sequence comprises an exogenous branch point comprising CCCTCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 10. In particular embodiments, the exogenous splice acceptor sequence comprises SEQ ID NO: 10.

In some embodiments, the exogenous splice acceptor sequence comprises an exogenous branch point comprising TCCCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 18. In particular embodiments, the exogenous splice acceptor sequence comprises SEQ ID NO: 18.

In certain embodiments, the exogenous splice acceptor sequence is not derived from intron 1 of the transferrin gene.

In some embodiments, the exogenous nucleic acid molecule comprises, from 5′ to 3′: (a) the exogenous splice acceptor sequence; (b) the first nucleic acid sequence; (c) an IRES sequence (SEQ ID NO: 29), a T2A sequence (SEQ ID NO: 30), a P2A sequence (SEQ ID NO: 31), an E2A sequence (SEQ ID NO: 32), or an F2A sequence (SEQ ID NO: 33); (d) a third nucleic acid sequence encoding a signal peptide; (e) the second nucleic acid sequence; and (f) the polyA signal. In some such embodiments, the signal peptide encoded by the third nucleic acid sequence comprises an amino acid sequence having at least 80%, at least 85%, at least 90%, at least 95%, or more, sequence identity to SEQ ID NO: 7. In particular embodiments, the signal peptide encoded by the third nucleic acid sequence comprises SEQ ID NO: 7. In some such embodiments, the signal peptide encoded by the third nucleic acid sequence comprises an amino acid sequence having at least 80%, at least 85%, at least 90%, at least 95%, or more, sequence identity to SEQ ID NO: 15. In particular embodiments, the signal peptide encoded by the third nucleic acid sequence comprises SEQ ID NO: 15.

In some embodiments, the polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

In some embodiments, the polyA signal comprises a nucleic acid sequence having at least 80%, at least 85%, at least 90%, at least 95%, or more, sequence identity to SEQ ID NO: 34. In certain embodiments, the polyA signal comprises a nucleic acid sequence of SEQ ID NO: 34.

In a particular embodiment of the pharmaceutical composition, the nucleic acid encoding the engineered nuclease is an mRNA. In certain embodiments, the mRNA is packaged within a lipid nanoparticle. In some embodiments, a viral vector comprises the template nucleic acid. In some such embodiments, the viral vector is an adenoviral vector, a lentiviral vector, a retroviral vector, or an adeno-associated viral (AAV) vector. In particular embodiments, a recombinant AAV vector comprises the template nucleic acid. In some such embodiments, the pharmaceutical composition comprises a therapeutically effective amount of: (a) an mRNA encoding the engineered nuclease, wherein the mRNA is packaged within a lipid nanoparticle; and (b) a recombinant AAV vector comprising the template nucleic acid. In some such embodiments, the recombinant AAV vector comprises a polynucleotide comprising, from 5′ to 3′, a first ITR, a template nucleic acid, and a second ITR.

In certain embodiments, the pharmaceutical composition comprises: (a) only one population of lipid nanoparticles comprising the mRNA molecule encoding the engineered nuclease; and (b) only one population of recombinant AAV vectors comprising the template nucleic acid. Such embodiments, using only one population of lipid nanoparticles comprising a nucleic acid molecule encoding the engineered nuclease, is distinguished from methods which require two or more populations of lipid nanoparticles to deliver unique mRNAs encoding unique proteins and/or nucleic acids which, when expressed together in a cell, act in concert to cleave a nuclease recognition sequence. For example, engineered TALEN nucleases and engineered zinc finger nucleases would typically require a plurality of populations of lipid nanoparticles, each comprising a different nucleic acid molecule encoding a different part of the functional engineered nuclease (i.e., a left TALEN and a right TALEN, or a left zinc finger nuclease and a right zinc finger nuclease).

In another embodiment, a first viral vector comprises the nucleic acid encoding the engineered nuclease. In particular embodiments, a first recombinant AAV vector comprises the nucleic acid encoding the engineered nuclease. In some such embodiments, the first recombinant AAV vector comprises a polynucleotide comprising, from 5′ to 3′, a first ITR, a nucleic acid encoding the engineered nuclease, and a second ITR. In some embodiments, the first recombinant AAV vector comprises a polynucleotide comprising, from 5′ to 3′, a first ITR, a promoter, a nucleic acid encoding the engineered nuclease, a polyA signal, and a second ITR, wherein the promoter is operably linked to (i.e., drives expression of) the engineered nuclease. In some such embodiments, a second viral vector comprises the template nucleic acid. In particular embodiments, a second recombinant AAV vector comprises the template nucleic acid. In some such embodiments, the second recombinant AAV vector comprises a polynucleotide comprising, from 5′ to 3′, a first ITR, a template nucleic acid, and a second ITR.

In some such embodiments, the pharmaceutical composition comprises only two populations of viral vectors, wherein a first population of viral vectors comprises the nucleic acid encoding the engineered nuclease, and wherein a second population of viral vectors comprises the template nucleic acid. Such embodiments, using only one population of viral vectors comprising a nucleic acid molecule encoding the engineered nuclease, is distinguished from methods which require two or more populations of viral vector to deliver unique nucleic acids encoding unique proteins and/or nucleic acids which, when expressed together in a cell, act in concert to cleave a nuclease recognition sequence. For example, engineered TALEN nucleases and engineered zinc finger nucleases would typically require a plurality of populations of viral vectors, each comprising a different nucleic acid molecule encoding a different part of the functional engineered nuclease (i.e., a left TALEN and a right TALEN, or a left zinc finger nuclease and a right zinc finger nuclease).

In some such embodiments, the pharmaceutical composition comprises: (a) a first population of recombinant AAV vectors comprising the nucleic acid encoding the engineered nuclease; and (b) a second population of recombinant AAV vectors comprising the template nucleic acid.

In some embodiments, the engineered nuclease is an engineered meganuclease, a TALEN, a compact TALEN, a megaTAL, a ZFN, or a CRISPR system nuclease.

In particular embodiments, the engineered nuclease is an engineered meganuclease having specificity for a meganuclease recognition sequence within intron 1 of the transferrin gene. In certain embodiments, the meganuclease recognition sequence comprises SEQ ID NO: 19 or 21. In some embodiments, the engineered nuclease is any engineered meganuclease disclosed herein.

In certain embodiments, the engineered nuclease is a TALEN having specificity for a TALEN recognition sequence within intron 1 of the transferrin gene.

In some embodiments, the engineered nuclease is a zinc finger nuclease having specificity for a zinc finger nuclease recognition sequence within intron 1 of the transferrin gene.

In some embodiments, the engineered nuclease is a CRISPR system nuclease having specificity for a recognition sequence within intron 1 of the transferrin gene.

In some embodiments, the polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

In another aspect, the invention provides a method for producing a genetically-modified eukaryotic cell comprising a modified transferrin gene, the method comprising introducing into a eukaryotic cell: (a) (i) a nucleic acid encoding an engineered nuclease having specificity for a recognition sequence within intron 1 of a transferrin gene, wherein the engineered nuclease is expressed in the eukaryotic cell, or (ii) an engineered nuclease protein having specificity for a recognition sequence within intron 1 of a transferrin gene; and (b) a template nucleic acid comprising an exogenous nucleic acid molecule, wherein the exogenous nucleic acid molecule comprises, from 5′ to 3′: (i) an exogenous splice acceptor sequence; (ii) a first nucleic acid sequence encoding a C-terminal fragment of a signal peptide; (iii) a second nucleic acid sequence encoding a polypeptide of interest; and (iv) a polyA signal; wherein the engineered nuclease produces a cleavage site at the recognition sequence, and wherein the exogenous nucleic acid molecule is inserted into intron 1 of the transferrin gene at the cleavage site, thereby generating the modified transferrin gene in the eukaryotic cell.

In particular embodiments of the method, upon generation of the modified transferrin gene, the endogenous promoter of the transferrin gene is operably linked to the exogenous nucleic acid molecule. In some embodiments, the endogenous promoter of the transferrin gene drives expression of the exogenous nucleic acid molecule.

In some embodiments of the method, the exogenous nucleic acid molecule further comprises a 5′ homology arm which is positioned 5′ upstream of the exogenous splice acceptor sequence, and a 3′ homology arm which is positioned 3′ downstream of the polyA signal, wherein the 5′ homology arm and the 3′ homology arm are homologous to sequences flanking the cleavage site, and wherein the exogenous nucleic acid molecule is inserted into the cleavage site by homologous recombination.

In one embodiment of the method, the first nucleic acid sequence is capable of being joined directly to the 3′ end of SEQ ID NO: 8 to generate a coding sequence for a human transferrin signal peptide having at least 80%, at least 85%, at least 90%, at least 95%, or more, sequence identity to SEQ ID NO: 7. In particular embodiments, the encoded transferrin signal peptide comprises SEQ ID NO: 7. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 9. In particular embodiments, the first nucleic acid sequence has at least 80%, at least 85%, at least 90%, at least 95%, or more, sequence identity to SEQ ID NO: 9, wherein the first two nucleotides of the first nucleic acid sequence are GG, GT, GA, or GC. In some embodiments, the first nucleic acid sequence comprises SEQ ID NO: 9.

In another embodiment of the method, the first nucleic acid sequence is capable of being joined directly to the 3′ end of SEQ ID NO: 16 to generate a coding sequence for a mouse transferrin signal peptide having at least 80%, at least 85%, at least 90%, at least 95%, or more, sequence identity to SEQ ID NO: 15. In particular embodiments, the encoded transferrin signal peptide comprises SEQ ID NO: 15. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 17. In particular embodiments, the first nucleic acid sequence has at least 80%, at least 85%, at least 90%, at least 95%, or more, sequence identity to SEQ ID NO: 17, wherein the first two nucleotides of the first nucleic acid sequence are GG, GT, GA, or GC. In some embodiments, the first nucleic acid sequence comprises SEQ ID NO: 17.

In particular embodiments of the method, the genetically-modified eukaryotic cell expresses a polypeptide comprising: (a) a transferrin signal peptide having at least 80%, at least 85%, at least 90%, at least 95%, or more, sequence identity to SEQ ID NO: 7 and (b) the polypeptide of interest; wherein the polypeptide of interest is secreted by the genetically-modified eukaryotic cell. In other particular embodiments of the method, the genetically-modified eukaryotic cell expresses a polypeptide comprising: (a) a transferrin signal peptide having at least 80%, at least 85%, at least 90%, at least 95%, or more, sequence identity to SEQ ID NO: 15 and (b) the polypeptide of interest; wherein the polypeptide of interest is secreted by the genetically-modified eukaryotic cell.

In some embodiments of the method, the exogenous splice acceptor sequence comprises an exogenous branch point comprising CCCTCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 10. In particular embodiments, the exogenous splice acceptor sequence comprises SEQ ID NO: 10.

In some embodiments of the method, the exogenous splice acceptor sequence comprises an exogenous branch point comprising TCCCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 18. In particular embodiments, the exogenous splice acceptor sequence comprises SEQ ID NO: 18.

In certain embodiments of the method, the exogenous splice acceptor sequence is not derived from intron 1 of the transferrin gene.

In some embodiments of the method, the exogenous nucleic acid molecule comprises, from 5′ to 3′: (a) the exogenous splice acceptor sequence; (b) the first nucleic acid sequence; (c) an IRES sequence (SEQ ID NO: 29), a T2A sequence (SEQ ID NO: 30), a P2A sequence (SEQ ID NO: 31), an E2A sequence (SEQ ID NO: 32), or an F2A sequence (SEQ ID NO: 33); (d) a third nucleic acid sequence encoding a signal peptide; (e) the second nucleic acid sequence; and (f) the polyA signal. In some such embodiments, the signal peptide encoded by the third nucleic acid sequence comprises an amino acid sequence having at least 80%, at least 85%, at least 90%, at least 95%, or more, sequence identity to SEQ ID NO: 7. In particular embodiments, the signal peptide encoded by the third nucleic acid sequence comprises SEQ ID NO: 7. In some such embodiments, the signal peptide encoded by the third nucleic acid sequence comprises an amino acid sequence having at least 80%, at least 85%, at least 90%, at least 95%, or more, sequence identity to SEQ ID NO: 15. In particular embodiments, the signal peptide encoded by the third nucleic acid sequence comprises SEQ ID NO: 15.

In some embodiments of the method, the polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

In some embodiments of the method, the polyA signal comprises a nucleic acid sequence having at least 80%, at least 85%, at least 90%, at least 95%, or more, sequence identity to SEQ ID NO: 34. In certain embodiments, the polyA signal comprises a nucleic acid sequence of SEQ ID NO: 34.

In one embodiment of the method, the nucleic acid encoding the engineered nuclease is an mRNA. In particular embodiments, the mRNA is packaged within a lipid nanoparticle.

In certain embodiments of the method, a viral vector comprises the template nucleic acid. In some such embodiments, the viral vector is an adenoviral vector, a lentiviral vector, a retroviral vector, or an adeno-associated viral (AAV) vector. In certain embodiments, a recombinant AAV vector comprises the template nucleic acid. In some such embodiments, the recombinant AAV vector comprises a polynucleotide comprising, from 5′ to 3′, a first ITR, a template nucleic acid, and a second ITR.

In some embodiments, the method comprises contacting the eukaryotic cell with: (a) a lipid nanoparticle comprising an mRNA encoding the engineered nuclease; and (b) a recombinant AAV vector comprising the template nucleic acid. In certain embodiments, the eukaryotic cell is contacted with: (a) only one population of lipid nanoparticles comprising the mRNA encoding the engineered nuclease; and (b) only one population of recombinant AAV vectors comprising the template nucleic acid. Such embodiments, using only one population of lipid nanoparticles comprising a nucleic acid molecule encoding the engineered nuclease, is distinguished from methods which require two or more populations of lipid nanoparticles to deliver unique mRNAs encoding unique proteins and/or nucleic acids which, when expressed together in a cell, act in concert to cleave a nuclease recognition sequence. For example, engineered TALEN nucleases and engineered zinc finger nucleases would typically require a plurality of populations of lipid nanoparticles, each comprising a different nucleic acid molecule encoding a different part of the functional engineered nuclease (i.e., a left TALEN and a right TALEN, or a left zinc finger nuclease and a right zinc finger nuclease).

In a particular embodiment of the method, a first viral vector comprises the nucleic acid encoding the engineered nuclease. In particular embodiments, a first recombinant AAV vector comprises the nucleic acid encoding the engineered nuclease. In some such embodiments, the first recombinant AAV vector comprises a polynucleotide comprising, from 5′ to 3′, a first ITR, a nucleic acid encoding the engineered nuclease, and a second ITR. In some embodiments, the first recombinant AAV vector comprises a polynucleotide comprising, from 5′ to 3′, a first ITR, a promoter, a nucleic acid encoding the engineered nuclease, a polyA signal, and a second ITR, wherein the promoter is operably linked to (i.e., drives expression of) the engineered nuclease. In some such embodiments, a second viral vector comprises the template nucleic acid. In particular embodiments, a second recombinant AAV vector comprises the template nucleic acid. In some such embodiments, the second recombinant AAV vector comprises a polynucleotide comprising, from 5′ to 3′, a first ITR, a template nucleic acid, and a second ITR.

In some such embodiments, the method comprises contacting the eukaryotic cell with only two populations of viral vectors, wherein a first population of viral vectors comprises the nucleic acid encoding the engineered nuclease, and wherein a second population of viral vectors comprises the template nucleic acid. Such embodiments, using only one population of viral vectors comprising a nucleic acid molecule encoding the engineered nuclease, is distinguished from methods which require two or more populations of viral vector to deliver unique nucleic acids encoding unique proteins and/or nucleic acids which, when expressed together in a cell, act in concert to cleave a nuclease recognition sequence. For example, engineered TALEN nucleases and engineered zinc finger nucleases would typically require a plurality of populations of viral vectors, each comprising a different nucleic acid molecule encoding a different part of the functional engineered nuclease (i.e., a left TALEN and a right TALEN, or a left zinc finger nuclease and a right zinc finger nuclease).

In some such embodiments of the method, the eukaryotic cell is contacted with: (a) a first population of recombinant AAV vectors comprising the nucleic acid encoding the engineered nuclease; and (b) a second population of recombinant AAV vectors comprising the template nucleic acid.

In some embodiments of the method, the engineered nuclease is an engineered meganuclease, a TALEN, a compact TALEN, a megaTAL, a ZFN, or a CRISPR system nuclease.

In some such embodiments of the method, the engineered nuclease is an engineered meganuclease having specificity for a recognition sequence within intron 1 of the transferrin gene. In particular embodiments, the meganuclease recognition sequence comprises SEQ ID NO: 19 or 21. In further embodiments, the engineered nuclease is any engineered meganuclease described herein.

In some embodiments of the method, the engineered nuclease is a TALEN having specificity for a TALEN recognition sequence within intron 1 of the transferrin gene.

In some embodiments of the method, the engineered nuclease is a zinc finger nuclease having specificity for a zinc finger nuclease recognition sequence within intron 1 of the transferrin gene.

In some embodiments of the method, the engineered nuclease is a CRISPR system nuclease having specificity for a recognition sequence within intron 1 of the transferrin gene.

In some embodiments of the method, the eukaryotic cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In another aspect, the invention provides a method for producing a genetically-modified cell in a mammalian subject, wherein the genetically-modified cell comprises a modified transferrin gene, the method comprising delivering to a target cell in the subject: (a) (i) a nucleic acid encoding an engineered nuclease having specificity for a recognition sequence within intron 1 of a transferrin gene, wherein the engineered nuclease is expressed in the target cell, or (ii) an engineered nuclease protein having specificity for a recognition sequence within intron 1 of a transferrin gene; and (b) a template nucleic acid comprising an exogenous nucleic acid molecule, wherein the exogenous nucleic acid molecule comprises, from 5′ to 3′: (i) an exogenous splice acceptor sequence; (ii) a first nucleic acid sequence encoding a C-terminal fragment of a signal peptide; (iii) a second nucleic acid sequence encoding a polypeptide of interest; and (iv) a polyA signal; wherein the engineered nuclease produces a cleavage site at the recognition sequence within intron 1 of the transferrin gene, and wherein the exogenous nucleic acid molecule is inserted into intron 1 of the transferrin gene at the cleavage site, thereby generating a modified transferrin gene in the target cell in the subject.

In some embodiments of the method, the exogenous nucleic acid molecule further comprises a 5′ homology arm which is positioned 5′ upstream of the exogenous splice acceptor sequence, and a 3′ homology arm which is positioned 3′ downstream of the polyA signal, wherein the 5′ homology arm and the 3′ homology arm are homologous to sequences flanking the cleavage site, and wherein the exogenous nucleic acid molecule is inserted into the cleavage site by homologous recombination.

In some embodiments of the method, upon generation of the modified transferrin gene, the endogenous promoter of the transferrin gene is operably linked to the exogenous nucleic acid molecule. In some embodiments, the endogenous promoter of the transferrin gene drives expression of the exogenous nucleic acid molecule.

In one embodiment of the method, the first nucleic acid sequence is capable of being joined directly to the 3′ end of SEQ ID NO: 8 to generate a coding sequence for a human transferrin signal peptide having at least 80%, at least 85%, at least 90%, at least 95%, or more, sequence identity to SEQ ID NO: 7. In particular embodiments, the encoded transferrin signal peptide comprises SEQ ID NO: 7. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 9. In particular embodiments, the first nucleic acid sequence has at least 80%, at least 85%, at least 90%, at least 95%, or more, sequence identity to SEQ ID NO: 9, wherein the first two nucleotides of the first nucleic acid sequence are GG, GT, GA, or GC. In some embodiments, the first nucleic acid sequence comprises SEQ ID NO: 9.

In another embodiment of the method, the first nucleic acid sequence is capable of being joined directly to the 3′ end of SEQ ID NO: 16 to generate a coding sequence for a mouse transferrin signal peptide having at least 80%, at least 85%, at least 90%, at least 95%, or more, sequence identity to SEQ ID NO: 15. In particular embodiments, the encoded transferrin signal peptide comprises SEQ ID NO: 15. In some embodiments, the first two nucleotides of the first nucleic acid sequence are GG, GT, GA, or GC, and the remaining nucleotides encode a polypeptide having at least 25%, at least 50%, at 75%, or 100% sequence identity to SEQ ID NO: 35. In certain embodiments, the first nucleic acid sequence has at least 80%, at least 85%, at least 90%, at least 95%, or more, sequence identity to SEQ ID NO: 17. In particular embodiments, the first nucleic acid sequence has at least 80%, at least 85%, at least 90%, at least 95%, or more, sequence identity to SEQ ID NO: 17, wherein the first two nucleotides of the first nucleic acid sequence are GG, GT, GA, or GC. In some embodiments, the first nucleic acid sequence comprises SEQ ID NO: 17.

In some embodiments of the method, the genetically-modified cell expresses a polypeptide comprising: (a) a transferrin signal peptide having at least 80%, at least 85%, at least 90%, at least 95%, or more, sequence identity to SEQ ID NO: 7; and (b) the polypeptide of interest; wherein the polypeptide of interest is secreted by the genetically-modified cell. In other particular embodiments of the method, the genetically-modified eukaryotic cell expresses a polypeptide comprising: (a) a transferrin signal peptide having at least 80%, at least 85%, at least 90%, at least 95%, or more, sequence identity to SEQ ID NO: 15 and (b) the polypeptide of interest; wherein the polypeptide of interest is secreted by the genetically-modified eukaryotic cell.

In some embodiments of the method, the exogenous splice acceptor sequence comprises an exogenous branch point comprising CCCTCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 10. In particular embodiments, the exogenous splice acceptor sequence comprises SEQ ID NO: 10.

In some embodiments of the method, the exogenous splice acceptor sequence comprises an exogenous branch point comprising TCCCAG. In some embodiments, the exogenous splice acceptor sequence comprises an exogenous splice acceptor site comprising AG. In some such embodiments, the exogenous splice acceptor sequence has at least 80%, at least 85%, at least 90%, at least 95%, or more, sequence identity to SEQ ID NO: 18. In particular embodiments, the exogenous splice acceptor sequence comprises SEQ ID NO: 18.

In certain embodiments of the method, the exogenous splice acceptor sequence is not derived from intron 1 of the transferrin gene.

In some embodiments of the method, the exogenous nucleic acid molecule comprises, from 5′ to 3′: (a) the exogenous splice acceptor sequence; (b) the first nucleic acid sequence; (c) an IRES sequence (SEQ ID NO: 29), a T2A sequence (SEQ ID NO: 30), a P2A sequence (SEQ ID NO: 31), an E2A sequence (SEQ ID NO: 32), or an F2A sequence (SEQ ID NO: 33); (d) a third nucleic acid sequence encoding a signal peptide; (e) the second nucleic acid sequence; and (f) the polyA signal. In some such embodiments, the signal peptide encoded by the third nucleic acid sequence comprises an amino acid sequence having at least 80%, at least 85%, at least 90%, at least 95%, or more, sequence identity to SEQ ID NO: 7. In particular embodiments, the signal peptide encoded by the third nucleic acid sequence comprises SEQ ID NO: 7. In some such embodiments, the signal peptide encoded by the third nucleic acid sequence comprises an amino acid sequence having at least 80%, at least 85%, at least 90%, at least 95%, or more, sequence identity to SEQ ID NO: 15. In particular embodiments, the signal peptide encoded by the third nucleic acid sequence comprises SEQ ID NO: 15.

In some embodiments of the method, the polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

In some embodiments of the method, the polyA signal comprises a nucleic acid sequence having at least 80%, at least 85%, at least 90%, at least 95%, or more, sequence identity to SEQ ID NO: 34. In certain embodiments, the polyA signal comprises a nucleic acid sequence of SEQ ID NO: 34.

In particular embodiments of the method, the nucleic acid encoding the engineered nuclease is an mRNA. In particular embodiments, the mRNA is packaged within a lipid nanoparticle. In some embodiments, a viral vector comprises the template nucleic acid. In some such embodiments, the viral vector is an adenoviral vector, a lentiviral vector, a retroviral vector, or an adeno-associated viral (AAV) vector. In particular embodiments, a recombinant AAV vector comprises the template nucleic acid. In some such embodiments, the recombinant AAV vector comprises a polynucleotide comprising, from 5′ to 3′, a first ITR, a template nucleic acid, and a second ITR.

In some embodiments, the method comprises delivering to the target cell: (a) a lipid nanoparticle comprising an mRNA encoding the engineered nuclease; and (b) a recombinant AAV vector comprising the template nucleic acid. In certain embodiments, the method comprises delivering to the target cell: (a) only one population of lipid nanoparticles comprising the mRNA encoding the engineered nuclease; or (b) only one population of recombinant AAV vectors comprising the template nucleic acid. Such embodiments, using only one population of lipid nanoparticles comprising a nucleic acid molecule encoding the engineered nuclease, is distinguished from methods which require two or more populations of lipid nanoparticles to deliver unique mRNAs encoding unique proteins and/or nucleic acids which, when expressed together in a cell, act in concert to cleave a nuclease recognition sequence. For example, engineered TALEN nucleases and engineered zinc finger nucleases would typically require a plurality of populations of lipid nanoparticles, each comprising a different nucleic acid molecule encoding a different part of the functional engineered nuclease (i.e., a left TALEN and a right TALEN, or a left zinc finger nuclease and a right zinc finger nuclease).

In another embodiment of the method, a first viral vector comprises the nucleic acid encoding the engineered nuclease. In particular embodiments, a first recombinant AAV vector comprises the nucleic acid encoding the engineered nuclease. In some such embodiments, the first recombinant AAV vector comprises a polynucleotide comprising, from 5′ to 3′, a first ITR, a nucleic acid encoding the engineered nuclease, and a second ITR. In some embodiments, the first recombinant AAV vector comprises a polynucleotide comprising, from 5′ to 3′, a first ITR, a promoter, a nucleic acid encoding the engineered nuclease, a polyA signal, and a second ITR, wherein the promoter is operably linked to (i.e., drives expression of) the engineered nuclease. In some such embodiments, a second viral vector comprises the template nucleic acid. In particular embodiments, a second recombinant AAV vector comprises the template nucleic acid. In some such embodiments, the second recombinant AAV vector comprises a polynucleotide comprising, from 5′ to 3′, a first ITR, the template nucleic acid, and a second ITR.

In some such embodiments, the method comprises delivering to the target cell only two populations of viral vectors, wherein a first population of viral vectors comprises the nucleic acid encoding the engineered nuclease, and wherein a second population of viral vectors comprises the template nucleic acid. Such embodiments, using only one population of viral vectors comprising a nucleic acid molecule encoding the engineered nuclease, is distinguished from methods which require two or more populations of viral vector to deliver unique nucleic acids encoding unique proteins and/or nucleic acids which, when expressed together in a cell, act in concert to cleave a nuclease recognition sequence. For example, engineered TALEN nucleases and engineered zinc finger nucleases would typically require a plurality of populations of viral vectors, each comprising a different nucleic acid molecule encoding a different part of the functional engineered nuclease (i.e., a left TALEN and a right TALEN, or a left zinc finger nuclease and a right zinc finger nuclease).

In some such embodiments, the method comprises delivering to the target cell: (a) a first population of recombinant AAV vectors comprising the nucleic acid encoding the engineered nuclease; and (b) a second population of recombinant AAV vectors comprising the template nucleic acid.

In some embodiments of the method, the engineered nuclease is an engineered meganuclease, a TALEN, a compact TALEN, a megaTAL a ZFN, or a CRISPR system nuclease.

In some embodiments of the method, the engineered nuclease is an engineered meganuclease having specificity for a meganuclease recognition sequence within intron 1 of the transferrin gene. In certain embodiments, the meganuclease recognition sequence comprises SEQ ID NO: 19 or 21. In some embodiments, the engineered nuclease is any engineered meganuclease of the invention.

In some embodiments of the method, the engineered nuclease is a TALEN having specificity for a TALEN recognition sequence within intron 1 of the transferrin gene.

In some embodiments of the method, the engineered nuclease is a zinc finger nuclease having specificity for a zinc finger nuclease recognition sequence within intron 1 of the transferrin gene.

In some embodiments of the method, the engineered nuclease is a CRISPR having specificity for a recognition sequence within intron 1 of the transferrin gene.

In some embodiments of the method, the cell is a mammalian cell. In some embodiments, the mammalian cell is selected from a human cell, non-human primate cell, or a mouse cell. In particular embodiments, the mammalian cell is a hepatocyte. In some embodiments, the hepatocyte is within the liver of a human, a non-human primate, or a mouse.

In another aspect, the invention provides a method for treating a disease in a subject in need thereof, the method comprising administering to the subject an effective amount of any of the pharmaceutical compositions disclosed herein.

In some embodiments of the method, the engineered nuclease produces a single or double strand break at a cleavage site of a recognition sequence within intron 1 of the transferrin gene, and wherein the exogenous nucleic acid molecule is inserted into intron 1 of the transferrin gene at the cleavage site, thereby generating a modified transferrin gene in the target cell in the subject.

In some embodiments, the method is effective to generate in the subject a genetically-modified target cell in vivo comprising a modified transferrin gene, wherein the modified transferrin gene comprises the exogenous nucleic acid molecule inserted within intron 1 of the transferrin gene. In some embodiments, upon generation of the modified transferrin gene, the endogenous promoter of the transferrin gene is operably linked to the exogenous nucleic acid molecule. In some embodiments, the endogenous promoter of the transferrin gene drives expression of the exogenous nucleic acid molecule.

In some embodiments of the method, the genetically-modified target cell expresses a polypeptide comprising: (a) a transferrin signal peptide having at least 80%, at least 85%, at least 90%, at least 95%, or more, sequence identity to SEQ ID NO: 7; and (b) the polypeptide of interest; wherein the polypeptide of interest is secreted by the genetically-modified target cell in vivo. In some embodiments of the method, the genetically-modified target cell expresses a polypeptide comprising: (a) a transferrin signal peptide having at least 80%, at least 85%, at least 90%, at least 95%, or more, sequence identity to SEQ ID NO: 15; and (b) the polypeptide of interest; wherein the polypeptide of interest is secreted by the genetically-modified target cell in vivo.

In some embodiments of the method, the polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

In particular embodiments of the method, the disease is Pompe disease, Fabry disease, Gaucher disease, Hunter syndrome, Marateaux-Lamy syndrome, Marquio A syndrome, lysosomal acid lipase deficiency, alpha-1-antitrypsin deficiency, adenosine deaminase deficiency, or Hurler syndrome. In particular embodiments, the method is effective to treat the disease.

In some embodiments, the method is effective to produce levels of the polypeptide of interest in the subject that are therapeutically beneficial or curative for the disease.

In another aspect, the present disclosure provides an engineered nuclease or a nucleic acid molecule encoding an engineered nuclease, such as an engineered meganuclease, TALEN nuclease, zinc finger nuclease, CRISPR system nuclease, compact TALEN, and/or megaTAL described herein for use as a medicament. The present disclosure further provides the use of an engineered nuclease or a nucleic acid molecule encoding an engineered nuclease described herein in the manufacture of a medicament for treating a disease in a subject in need thereof. In one such embodiment, the medicament is useful in the treatment of Pompe disease, Fabry disease, Gaucher disease, Hunter syndrome, Marateaux-Lamy syndrome, Marquio A syndrome, lysosomal acid lipase deficiency, alpha-1-antitrypsin deficiency, adenosine deaminase deficiency, or Hurler syndrome. In some embodiments, the engineered nuclease or a nucleic acid molecule encoding an engineered nuclease described herein is useful for manufacturing a medicament for producing levels of the polypeptide of interest in the subject that are therapeutically beneficial or curative for the disease.

### BRIEF DESCRIPTION OF THE FIGURES

FIGS. 1A and 1B. Schematic of the region surrounding intron 1 of a transferrin gene and exemplary exogenous nucleic acid molecules. The region surrounding intron 1 of a transferrin gene includes an upstream endogenous transferrin promoter, exon 1, which encodes an N-terminal fragment of the transferrin signal peptide, an endogenous splice donor site at the 5′ end of intron 1, an endogenous splice acceptor site at the 3′ end of intron 1, and exon 2 which encodes the C-terminal fragment of the transferrin signal peptide. The transferrin gene is modified using any of the nucleases of the invention to introduce an exogenous nucleic acid molecule encoding a polypeptide of interest. As shown in FIG. 1A, in some instances, the exogenous nucleic acid molecule includes an exogenous splice acceptor sequence, a sequence encoding the C-terminal fragment of the transferrin signal peptide, a sequence encoding a polypeptide of interest, and a polyA signal. As shown in FIG. 1B, in other instances, the exogenous nucleic acid molecule may include an exogenous splice acceptor sequence, a sequence encoding the C-terminal fragment of the transferrin signal peptide, a skipping sequence such as a 2A sequence or IRES sequence, a sequence encoding a full-length signal peptide, a sequence encoding a polypeptide of interest, and a polyA signal.

FIG. 2. TFN 3-4 and TFN 19-20 meganuclease recognition sequences in intron 1 of the human transferrin gene. Each recognition sequence targeted by a recombinant meganuclease of the invention comprises two recognition half-sites. Each recognition half-site comprises 9 base pairs, separated by a 4 base pair central sequence. The TFN 3-4 recognition sequence (SEQ ID NO: 19) is located in intron 1 of the human transferrin gene (SEQ ID NO: 4) and comprises two recognition half-sites referred to as TFN3 and TFN4. The TFN 19-20 recognition sequence (SEQ ID NO: 21) is located in intron 1 of the mouse transferrin gene (SEQ ID NO: 12) and comprises two recognition half-sites referred to as TFN19 and TFN20.

FIG. 3. The recombinant meganucleases of the invention comprise two subunits, wherein the first subunit comprising the HVR1 region binds to a first recognition half-site (e.g., TFN3 or TFN19) and the second subunit comprising the HVR2 region binds to a second recognition half-site (e.g., TFN4 or TFN20). In embodiments where the recombinant meganuclease is a single-chain meganuclease, the first subunit comprising the HVR1 region is positioned as either the N-terminal or C-terminal subunit. Likewise, the second subunit comprising the HVR2 region is positioned as either the N-terminal or C-terminal subunit.

FIG. 4. Schematic of a reporter assay in CHO cells for evaluating recombinant meganucleases targeting recognition sequences found in intron 1 of the transferrin gene (SEQ ID NO: 4 or SEQ ID NO: 12). For the engineered meganucleases described herein, a CHO cell line was produced in which a reporter cassette was integrated stably into the genome of the cell. The reporter cassette comprised, in 5′ to 3′ order: an SV40 Early Promoter; the 5′ 2/3 of the GFP gene; the recognition sequence for an engineered meganuclease of the invention (e.g., the TFN 3-4 recognition sequence or the TFN 19-20 recognition sequence); the recognition sequence for the CHO-23/24 meganuclease (WO/2012/167192); and the 3′ 2/3 of the GFP gene. Cells stably transfected with this cassette did not express GFP in the absence of a DNA break-inducing agent. Meganucleases were introduced by transduction of plasmid DNA or mRNA encoding each meganuclease. When a DNA break was induced at either of the meganuclease recognition sequences, the duplicated regions of the GFP gene recombined with one another to produce a functional GFP gene. The percentage of GFP-expressing cells could then be determined by flow cytometry as an indirect measure of the frequency of genome cleavage by the meganucleases.

FIGS. 5A and 5B. Efficiency of recombinant meganucleases for recognizing and cleaving recognition sequences in intron 1 of the human transferrin gene (SEQ ID NO: 4) or the mouse transferrin gene (SEQ ID NO: 12) in a CHO cell reporter assay. The TFN 3-4x.2 recombinant meganuclease set forth in SEQ ID NO: 23 was engineered to target the TFN 3-4 recognition sequence (SEQ ID NO: 19), and the TFN 19-20x.76 recombinant meganuclease set forth in SEQ ID NO: 26 was engineered to target the TFN 19-20 recognition sequence (SEQ ID NO: 21). Each meganuclease was screened for efficacy in the CHO cell reporter assay. The results shown provide the percentage of GFP-expressing cells observed in each assay, which indicates the efficacy of each meganuclease for cleaving a TFN target recognition sequence or the CHO-23/24 recognition sequence. A negative control (TFN 3-4 bs or TFN 19-20 bs) was further included in each assay. A) Meganucleases targeting the TFN 3-4 recognition sequence. B) Meganucleases targeting the TFN 19-20 recognition sequence.

FIGS. 6A and 6B. Time course of engineered meganuclease efficacy in the CHO cell reporter assay. The TFN 3-4x.2 (SEQ ID NO: 23) and TFN 19-20x.76 (SEQ ID NO: 26) meganucleases were evaluated in the CHO reporter assay with the percentage of GFP-expressing cells determined 2, 5, and 7 days after introduction of meganuclease-encoding mRNA into the CHO reporter cells. A CHO 23/24 meganuclease was also included at each time point as a positive control. A) Results of CHO cell reporter assay with the TFN 3-4x.2 meganuclease and positive control. B) Results of CHO cell reporter assay with the TFN 19-20x.76 meganuclease.

FIGS. 7A and 7B. Schematics showing the design of repair donor plasmids. Each repair plasmid included a repair cassette comprising, from 5′ to 3′: a 5′ (left) homology arm, a splice acceptor sequence derived from intron 1 of the mouse transferrin gene, a sequence encoding the C-terminal fragment of the transferrin signal peptide found in exon 2, a sequence encoding a SEAP reporter transgene, an SV40 polyA signal, and a 3′ (right) homology arm. Additionally, each repair cassette was flanked by two target sequences for the TFN 19-20 meganuclease to allow the plasmid to be cleaved and the repair cassette to be linearized for introduction to cells. One repair construct (Repair TFN T2A SEAP) further included a T2A ribosome-skipping peptide sequence and a full transferrin signal peptide sequence before the SEAP transgene (FIG. 7A). A second repair construct (Repair TFN SEAP) without a T2A or full signal peptide sequence was also included (FIG. 7B). Each repair donor plasmid further comprised a nuclease cassette comprising a JeT promoter, a sequence encoding either the TFN 19-20x.76 meganuclease or a truncated, nonfunctional TFN 19-20x.76 nuclease (truncTFN 19-20), and an SV40 polyA signal.

FIG. 8. Secreted embryonic alkaline phosphatase (SEAP) expression from the transferrin intron 1 locus. FL83B (murine liver cell line) cells were electroporated with repair constructs and assayed for SEAP expression in the conditioned media. Both repair constructs that included the TFN 19-20x.76 nuclease yielded SEAP expression by ten days post electroporation.

FIG. 9. PCR verification of insertion of the SEAP transgene into the transferrin intron 1. The forward primer anneals to chromosomal sequence outside of the homology arm and the reverse primer anneals to the SEAP sequence.

FIG. 10. Schematic showing the design of AAV vectors for in vivo studies. A) A donor AAV (referred to as “Repair A AAV”) contained a repair cassette comprising, from 5′ to 3′: a 5′ inverted terminal repeat (ITR), a 5′ (left) homology arm, a splice acceptor sequence derived from intron 1 of the mouse transferrin gene, a sequence encoding the C-terminal fragment of the transferrin signal peptide found in exon 2, a T2A sequence, a full transferrin signal peptide sequence, a sequence encoding a SEAP reporter transgene, an SV40 polyA signal, a 3′ (right) homology arm, and a 3′ ITR. B) A second AAV encoding the TFN 19-20x.76 meganuclease driven by a TBG promoter was also prepared (referred to as “TFN 19-20 AAV”) using a vector comprising, from 5′ to 3′: a 5′ ITR, a TBG promoter, a sequence encoding the TFN 19-20x.76 meganuclease, a WPRE sequence, a bHG polyA signal, and a 3′ ITR.

FIG. 11. Secreted SEAP expression from the transferrin locus. FVB mice were dosed with 2.7×10¹² vg/mouse of the repair A AAV in combination with 2.7×10¹¹ vg/mouse of the TFN 19-20 AAV. Additionally, two control cohorts were included. One control cohort was injected with PBS alone while the other cohort received the same amount of the repair A AAV alone.

FIG. 12. Secreted SEAP expression from the transferrin locus. FVB mice were dosed with 3.0×10¹² vg/mouse of the repair A AAV in combination with 3.0×10¹¹ vg/mouse of the TFN 19-20 AAV. Additionally, two control cohorts were included. One control cohort was injected with 3.0×10¹² vg/mouse of the repair A AAV construct alone while the other cohort received 3.0×10¹¹ vg/mouse of the TFN 19-20 AAV alone.

FIG. 13. SEAP insertion frequency at the TFN 19-20 recognition sequence. Mice were administered 2.7×10¹² vg/mouse of the repair A AAV construct with 2.7×10¹¹ vg/mouse of the TFN19-20 AAV or PBS as a control. Each bar represents independent mice in the experiment. If no bars are present, then a mouse was not injected.

FIG. 14. SEAP insertion frequency at the TFN 19-20 recognition sequence. Mice were administered 3.0×10¹² vg/mouse of the repair A AAV construct with 3.0×10¹¹ vg/mouse of the TFN19-20 AAV or 3.0×10¹¹ vg/mouse of the TFN 19-20 AAV alone as a control. Each bar represents independent mice in the experiment. If no bars are present, then a mouse was not injected.

FIG. 15. Percent insertion or deletion (indels) at the TFN 19-20 recognition sequence. Mice were administered 2.7×10¹² vg/mouse of the repair A AAV construct with 2.7×10¹¹ vg/mouse of the TFN19-20 AAV or 2.7×10¹² vg/mouse of the repair A AAV alone or PBS as controls. Each bar represents independent mice in the experiment. If no bars are present, then a mouse was not injected.

FIG. 16. Percent indels at the TFN 19-20 recognition sequence. Mice were administered 3.0×10¹² vg/mouse of the repair A AAV construct with 3.0×10¹¹ vg/mouse of the TFN19-20 AAV or 3.0×10¹² vg/mouse of the repair A AAV or PBS as controls. Each bar represents independent mice in the experiment. If no bars are present, then a mouse was not injected.

FIG. 17. Schematic showing the design of AAV vectors for in vivo studies. A) A donor AAV (referred to as “Repair B”) contained a repair cassette comprising, from 5′ to 3′: a 5′ inverted terminal repeat (ITR), a first inverted TFN 19-20 recognition sequence, a 5′ (left) homology arm, a splice acceptor sequence derived from intron 1 of the mouse transferrin gene, a sequence encoding the C-terminal fragment of the transferrin signal peptide found in exon 2, a T2A sequence, a full transferrin signal peptide sequence, a sequence encoding a SEAP reporter transgene, an SV40 polyA signal, a 3′ (right) homology arm, a second inverted TFN 19-20 recognition sequence, and a 3′ ITR. B) A second AAV encoding the TFN 19-20x.76 meganuclease driven by a TBG promoter was also prepared (referred to as TFN 19-20) using a vector comprising, from 5′ to 3′: a 5′ ITR, a TBG promoter, a sequence encoding the TFN 19-20x.76 meganuclease, a WPRE sequence, a bHG polyA signal, and a 3′ ITR.

FIG. 18. Schematic showing the design of AAV vectors for in vivo studies. A) A donor AAV (referred to as “Repair C”) contained a repair cassette comprising, from 5′ to 3′: a 5′ inverted terminal repeat (ITR), a 5′ (left) homology arm, a splice acceptor sequence derived from intron 1 of the mouse transferrin gene, a sequence encoding the C-terminal fragment of the transferrin signal peptide found in exon 2, a sequence encoding a SEAP reporter transgene, an SV40 polyA signal, a 3′ (right) homology arm, a second inverted TFN 19-20 recognition sequence and a 3′ ITR. B) A second AAV encoding the TFN 19-20x.76 meganuclease driven by a TBG promoter was also prepared (referred to as TFN 19-20) using a vector comprising, from 5′ to 3′: a 5′ ITR, a TBG promoter, a sequence encoding the TFN 19-20x.76 meganuclease, a WPRE sequence, a bHG polyA signal, and a 3′ ITR.

FIG. 19. Schematic showing the design of AAV vectors for in vivo studies. A) A donor AAV (referred to as “Repair D”) contained a repair cassette comprising, from 5′ to 3′: a 5′ inverted terminal repeat (ITR), a first inverted TFN 19-20 recognition sequence, a 5′ (left) homology arm, a splice acceptor sequence derived from intron 1 of the mouse transferrin gene, a sequence encoding the C-terminal fragment of the transferrin signal peptide found in exon 2, a sequence encoding a SEAP reporter transgene, an SV40 polyA signal, a 3′ (right) homology arm, a second inverted TFN 19-20 recognition sequence, and 3′ ITR. B) A second AAV encoding the TFN 19-20x.76 meganuclease driven by a TBG promoter was also prepared (referred to as TFN 19-20) using a vector comprising, from 5′ to 3′: a 5′ ITR, a TBG promoter, a sequence encoding the TFN 19-20x.76 meganuclease, a WPRE sequence, a bHG polyA signal, and a 3′ ITR.

FIG. 20. Secreted SEAP expression from the transferrin locus. FVB mice were dosed with 3.0×10¹² vg/mouse of either repair A, repair B, repair C, or repair D AAV in combination with 3.0×10¹¹ vg/mouse of the TFN 19-20 AAV. Additionally, two control cohorts were included. One control cohort was injected with the same amount of the TFN 19-20 AAV alone, and a second control cohort was injected with PBS.

FIG. 21. SEAP insertion frequency at the TFN 19-20 recognition sequence. Mice were administered 3.0×10¹² vg/mouse of either the repair A, repair B, repair C, or repair D AAV with 3.0×10¹¹ vg/mouse of the TFN19-20 AAV. Each bar represents independent mice in the experiment. If no bars are present, then a mouse was not injected.

FIG. 22. Percent indels at the TFN 19-20 recognition sequence. Mice were administered 3.0×10¹² vg/mouse of either the repair A, repair B, repair C, or repair D AAV with 3.0×10¹¹ vg/mouse of the TFN19-20 AAV or the TFN 19-20 AAV or PBS as controls. Each bar represents independent mice in the experiment. If no bars are present, then a mouse was not injected.

### BRIEF DESCRIPTION OF THE SEQUENCES

SEQ ID NO: 1 sets forth the amino acid sequence of the wild-type I-CreI meganuclease from Chlamydomonas reinhardtii.

SEQ ID NO: 2 sets forth the amino acid sequence of the LAGLIDADG motif.

SEQ ID NO: 3 sets forth the nucleic acid sequence of exon 1 of the human transferrin gene.

SEQ ID NO: 4 sets forth the nucleic acid sequence of intron 1 of the human transferrin gene.

SEQ ID NO: 5 sets forth the nucleic acid sequence of exon 2 of the human transferrin gene.

SEQ ID NO: 6 sets forth the nucleic acid sequence encoding the human transferrin gene signal peptide.

SEQ ID NO: 7 sets forth the amino acid sequence of the human transferrin signal peptide.

SEQ ID NO: 8 sets forth the nucleic acid sequence encoding a signal peptide fragment in exon 1 of the human transferrin gene.

SEQ ID NO: 9 sets forth the nucleic acid sequence encoding a signal peptide fragment in exon 2 of the human transferrin gene.

SEQ ID NO: 10 sets forth the nucleic acid sequence of the endogenous human transferrin gene splice acceptor sequence.

SEQ ID NO: 11 sets forth the nucleic acid sequence of exon 1 of the mouse transferrin gene.

SEQ ID NO: 12 sets forth the nucleic acid sequence of intron 1 of the mouse transferrin gene.

SEQ ID NO: 13 sets forth the nucleic acid sequence of exon 2 of the mouse transferrin gene.

SEQ ID NO: 14 sets forth the nucleic acid sequence encoding the mouse transferrin gene signal peptide.

SEQ ID NO: 15 sets forth the amino acid sequence of the mouse transferrin signal peptide.

SEQ ID NO: 16 sets forth the nucleic acid sequence encoding a signal peptide fragment in exon 1 of the mouse transferrin gene.

SEQ ID NO: 17 sets forth the nucleic acid sequence encoding a signal peptide fragment in exon 2 of the mouse transferrin gene.

SEQ ID NO: 18 sets forth the nucleic acid sequence of the endogenous mouse transferrin gene splice acceptor sequence.

SEQ ID NO: 19 sets forth the nucleic acid sequence of the TFN 3-4 recognition sequence (sense strand).

SEQ ID NO: 20 sets forth the nucleic acid sequence of the TFN 3-4 recognition sequence (antisense strand).

SEQ ID NO: 21 sets forth the nucleic acid sequence of the TFN 19-20 recognition sequence (sense strand).

SEQ ID NO: 22 sets forth the nucleic acid sequence of the TFN 19-20 recognition sequence (antisense strand).

SEQ ID NO: 23 sets forth the amino acid sequence of the TFN 3-4x.2 meganuclease.

SEQ ID NO: 24 sets forth the amino acid sequence of the TFN3 half-site-binding subunit of the TFN 3-4x.2 meganuclease.

SEQ ID NO: 25 sets forth the amino acid sequence of the TFN4 half-site-binding subunit of the TFN 3-4x.2 meganuclease.

SEQ ID NO: 26 sets forth the amino acid sequence of the TFN 19-20x.76 meganuclease.

SEQ ID NO: 27 sets forth the amino acid sequence of the TFN19 half-site-binding subunit of the TFN 19-20x.76 meganuclease.

SEQ ID NO: 28 sets forth the amino acid sequence of the TFN20 half-site-binding subunit of the TFN 19-20x.76 meganuclease.

SEQ ID NO: 29 sets forth the nucleic acid sequence of an internal ribosome entry site (IRES).

SEQ ID NO: 30 sets forth the nucleic acid sequence encoding the T2A peptide.

SEQ ID NO: 31 sets forth the nucleic acid sequence encoding the P2A peptide.

SEQ ID NO: 32 sets forth the nucleic acid sequence encoding the E2A peptide.

SEQ ID NO: 33 sets forth the nucleic acid sequence encoding the F2A peptide.

SEQ ID NO: 34 sets forth the nucleic acid sequence encoding a polyA signal.

SEQ ID NO: 35 sets forth the amino acid sequence LCLA.

SEQ ID NO: 36 sets forth the nucleic acid sequence for the P1 primer of Example 2.

SEQ ID NO: 37 sets forth the nucleic acid sequence for the P2 primer of Example 2.

SEQ ID NO: 38 sets forth the nucleic acid sequence for the binding site forward primer of Example 4.

SEQ ID NO: 39 sets forth the nucleic acid sequence for the binding site reverse primer of Example 4.

SEQ ID NO: 40 sets forth the nucleic acid sequence for the binding site probe of Example 4.

SEQ ID NO: 41 sets forth the nucleic acid sequence for the HDR forward primer of Example 4.

SEQ ID NO: 42 sets forth the nucleic acid sequence for the HDR reverse primer of Example 4.

SEQ ID NO: 43 sets forth the nucleic acid sequence for the HDR probe of Example 4.

SEQ ID NO: 44 sets forth the nucleic acid sequence for the indel reference forward primer of Example 4.

SEQ ID NO: 45 sets forth the nucleic acid sequence for the indel reference reverse primer of Example 4.

SEQ ID NO: 46 sets forth the nucleic acid sequence for the indel reference probe of Example 4.

SEQ ID NO: 47 sets forth the nucleic acid sequence for the HDR reference forward primer of Example 4.

SEQ ID NO: 48 sets forth the nucleic acid sequence for the HDR reference reverse primer of Example 4.

SEQ ID NO: 49 sets forth the nucleic acid sequence for the HDR reference probe of Example 4.

SEQ ID NO: 50 sets forth the nucleic acid sequence of a peptide linker sequence.

SEQ ID NO: 51 sets forth the nucleic acid sequence of a plasmid cassette including repair cassette and nuclease expression cassette.

SEQ ID NO: 52 sets forth the nucleic acid sequence of a plasmid cassette including repair cassette and nuclease expression cassette.

SEQ ID NO: 53 sets forth the nucleic acid sequence of a repair donor plasmid cassette (repair A).

SEQ ID NO: 54 sets forth the nucleic acid sequence of a repair donor plasmid cassette (repair B).

SEQ ID NO: 55 sets forth the nucleic acid sequence of a repair donor plasmid cassette (repair C).

SEQ ID NO: 56 sets forth the nucleic acid sequence of a repair donor plasmid cassette (repair D).

SEQ ID NO: 57 sets forth the nucleic acid sequence of a nuclease expression cassette.

SEQ ID NO: 58 sets forth the nucleic acid sequence of an inverted TFN 3-4 recognition sequence (sense strand).

SEQ ID NO: 59 sets forth the nucleic acid sequence of an inverted TFN 3-4 recognition sequence (antisense strand).

SEQ ID NO: 60 sets forth the nucleic acid sequence of an inverted TFN 19-20 recognition sequence (sense strand).

SEQ ID NO: 61 sets forth the nucleic acid sequence of an inverted TFN 19-20 recognition sequence (antisense strand).

SEQ ID NO: 62 sets forth the nucleic acid sequence of an internal ribosome entry site (IRES).

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein, are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “exogenous” or “heterologous” in reference to a nucleotide sequence or amino acid sequence is intended to mean a sequence that is purely synthetic, that originates from a foreign species, or, if from the same species, is substantially modified from its native form in composition and/or genomic locus by deliberate human intervention.

As used herein, the term “endogenous” in reference to a nucleotide sequence or protein is intended to mean a sequence or protein that is naturally comprised within or expressed by a cell.

As used herein, the terms “nuclease” and “endonuclease” are used interchangeably to refer to naturally-occurring or engineered enzymes, which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be, for example, an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of meganuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two meganuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053. In some embodiments, a linker may have at least 80%, at least 85%, at least 90%, at least 91%, at least 92%, at least 93%, at least 94%, at least 95%, at least 96%, at least 97%, at least 98%, at least 99%, or more, sequence identity to SEQ ID NO: 50, which sets forth residues 154-195 of SEQ ID NOs: 23 or 26. In some embodiments, a linker may have an amino acid sequence comprising SEQ ID NO: 50, which sets forth residues 154-195 of any one of SEQ ID NOs: 23 or 26.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, 51 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12^{th} and 13^{th} amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, End1, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the term “megaTAL” refers to a single-chain endonuclease comprising a transcription activator-like effector (TALE) DNA binding domain with an engineered, sequence-specific homing endonuclease.

As used herein, the term “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, 51 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein), which bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation.

As used herein, with respect to a protein, the term “recombinant” or “engineered” means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from engineered or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion or substitution of an amino acid residue in the recombinant sequence relative to a reference sequence (e.g., a wild-type or a native sequence).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized and bound by the I-TevI domain, followed by a nonspecific spacer 4-16 basepairs in length, followed by a second sequence 16-22 bp in length that is recognized and bound by the TAL-effector domain (this sequence typically has a 5′ T base). Cleavage by a compact TALEN produces two basepair 3′ overhangs. In the case of a CRIPSR system nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRIPSR system nuclease can produce blunt ends (such as by a class 2, type II CRISPR nuclease) or overhanging ends (such as by a class 2, type V CRISPR nuclease), depending on the CRISPR nuclease. In those embodiments wherein a Cpf1 CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence length requirements for the PAM and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize and bind to an 18-bp recognition sequence comprising a pair of nine basepair “half-sites” separated by a 2-10 basepair “spacer” sequence, and cleavage by the nuclease (i.e., a left zinc finger and a right zinc finger pair) creates a blunt end or a 5′ overhang of variable length (frequently four basepairs).

As used herein, the term “target site” or “target sequence” refers to a region of the chromosomal DNA of a cell comprising a recognition sequence for a nuclease.

As used herein, the term “DNA-binding affinity” or “binding affinity” means the tendency of a nuclease to non-covalently associate with a reference DNA molecule (e.g., a recognition sequence or an arbitrary sequence). Binding affinity is measured by a dissociation constant, K_{d}. As used herein, a nuclease has “altered” binding affinity if the K_{d} of the nuclease for a reference recognition sequence is increased or decreased by a statistically significant percent change relative to a reference nuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

As used herein, a nuclease has “altered” specificity if it binds to and cleaves a recognition sequence, which is not bound to and cleaved by a reference nuclease (e.g., a wild-type) under physiological conditions, or if the rate of cleavage of a recognition sequence is increased or decreased by a biologically significant amount (e.g., at least 2×, or 2×-10×) relative to a reference nuclease.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at the target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function.

As used herein, “homology arms” or “sequences homologous to sequences flanking a meganuclease cleavage site” refer to sequences flanking the 5′ and 3′ ends of a nucleic acid molecule which promote insertion of the nucleic acid molecule into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein with respect to both amino acid sequences and nucleic acid sequences, the terms “percent identity,” “sequence identity,” “percentage similarity,” “sequence similarity,” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment and despite the fact that X and Y may be different numbers.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease, or by a monomer of a TALEN or zinc finger nuclease.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of any one of SEQ ID NOs: 23 or 26. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity. In some embodiments, variable residues within a hypervariable region correspond to one or more of positions 24, 26, 28, 30, 32, 33, 38, 40, 42, 44, 46, 68, 70, 75, and 77 of any one of SEQ ID NOs: 23 or 26. In other embodiments, variable residues within a hypervariable region correspond to one or more of positions 215, 217, 219, 221, 223, 224, 229, 231, 233, 235, 237, 259, 261, 266, and 268 of any one of SEQ ID NOs: 23 or 26. In particular embodiments, variable residues can include position 41 of SEQ ID NO: 23.

As used herein, a “transferrin gene” refers to a gene encoding a polypeptide having an ability to bind iron, including a mammalian transferrin, such as the human transferrin protein (Swiss-Prot accession number P02787); murine transferrin protein (GenBank accession AAL34533.1); or macaque transferrin protein (GenBank accession ACB11584.1). Exemplary transferrin genes include the human transferrin gene (NCBI Gene No. 7018), the murine transferrin gene (NCBI Gene No. 22041), and the rhesus transferrin gene (NCBI Gene No. 100426895). The term transferrin gene also refers to naturally occurring DNA sequence variations of a transferrin gene, such as a single nucleotide polymorphism (SNP). Exemplary SNPs may be found through the publicly accessible National Center for Biotechnology Information dbSNP Short Genetic Variations database.

As used herein, the term “operably linked” refers to a functional linkage between an endogenous promoter of a transferrin gene (e.g., the human transferrin promoter) and an exogenous nucleic acid sequence positioned within intron 1 of the transferrin gene, such that the endogenous transferrin promoter controls expression of the exogenous nucleic acid sequence.

As used herein, the term “is capable of being joined directly” or “joined directly” refers to the positioning of two nucleic acid sequences directly adjacent to one another following the splicing of intervening intron sequences, such that the two sequences are transcribed in-frame as a single transcript to generate a polypeptide (e.g., a signal peptide). By way of example, an exogenous nucleic acid sequence can be inserted into intron 1 of the transferrin gene, wherein the exogenous nucleic acid sequence comprises, from 5′ to 3′, an exogenous splice acceptor sequence, a first nucleic acid sequence encoding a C-terminal fragment of a signal peptide, a second nucleic acid sequence encoding a polypeptide of interest, and a polyA signal. In such an example, during transcription, the intervening endogenous intron sequence and the inserted exogenous splice acceptor sequence will be spliced out, such that exon 1 of the transferrin gene, which encodes the N-terminal fragment of the signal peptide, is joined directly to the first nucleic acid sequence encoding the C-terminal fragment of the signal peptide, such that the two fragments can be transcribed in-frame as a single transcript to generate a signal peptide.

As used herein, the term “template nucleic acid” or “repair template” refers to a nucleic acid construct (e.g., an mRNA or a vector) comprising an exogenous nucleic acid molecule of the invention. The template nucleic acid may include additional nucleic acid sequences (e.g., nucleic acids encoding promoters or replication sequences) that are not part of the exogenous nucleic acid molecule inserted within intron 1 of a transferrin gene. In some embodiments, the template nucleic acid comprises an exogenous splice acceptor sequence; a first nucleic acid sequence encoding a C-terminal fragment of a signal peptide; (c) a second nucleic acid sequence encoding an exogenous polypeptide of interest; and (d) a polyA signal. In some other embodiments, the template nucleic acid comprises (a) an exogenous splice acceptor sequence; (b) a first nucleic acid sequence encoding a C-terminal fragment of a signal peptide; (c) a 2A sequence or IRES sequence; (d) a third nucleic acid sequence encoding a signal peptide; (e) a second nucleic acid sequence encoding an exogenous polypeptide of interest; and (f) a poly A signal. In such embodiments, the template nucleic acid can include 5′ and 3′ homology arms that are positioned 5′ and 3′ relative to the exogenous splice acceptor sequence and poly A signal. In other such embodiments, the template nucleic acid can further include one or more nuclease recognition sequences described herein that are 5′ and 3′ relative to the 5′ and 3′ homology arms. Exemplary and non-limiting template nucleic acid sequences are provided as SEQ ID NOs: 51-56.

As used herein, the term “exogenous nucleic acid” or “exogenous nucleic acid molecule” refers to a nucleic acid that does not naturally occur within intron 1 of the transferrin gene but is capable of being inserted within intron 1 at an engineered nuclease cleavage site, as described herein. Exogenous nucleic acid molecules of the invention can comprise, for example: (a) an exogenous splice acceptor sequence; (b) a first nucleic acid sequence encoding a C-terminal fragment of a signal peptide; (c) a second nucleic acid sequence encoding a polypeptide of interest; and (d) a polyA signal. The exogenous nucleic acid may include additional elements, such as an IRES sequence (SEQ ID NO: 29) or a 2A sequence (e.g., T2A (SEQ ID NO: 30), P2A (SEQ ID NO: 31), E2A (SEQ ID NO: 32), or F2A (SEQ ID NO: 33)), and/or a sequence encoding a full-length signal peptide, such as a full-length transferrin signal peptide (e.g., SEQ ID NO: 7 or SEQ ID NO: 15).

As used herein, the term “exogenous splice acceptor sequence” refers to a nucleotide sequence comprising an exogenous branch point sequence and an exogenous splice acceptor site sequence. In such sequences, the exogenous branch point is 5′ upstream of the exogenous splice acceptor site and enables the exogenous splice acceptor site to join with the endogenous splice donor site in intron 1 of the transferrin gene. The exogenous branch point and the exogenous splice acceptor site can be separated by a number of nucleotides or, alternatively, can be adjacent to one another. The exogenous branch point and/or the exogenous splice acceptor site can be derived from intron 1 of a transferrin gene but be located in a position at which it is not naturally found in the transferrin gene. Alternatively, the exogenous branch point and/or the exogenous splice acceptor site can be derived from a gene other than the transferrin gene. In some embodiments, the exogenous splice acceptor sequence may comprise the branch point (CCCTCAG) and/or splice acceptor site (AG) found in intron 1 of the human transferrin gene. In other embodiments, the exogenous splice acceptor sequence may comprise the branch point (TCCCAG) and/or splice acceptor site (AG) found in intron 1 of the mouse transferrin gene. Branch point, splice donor, and splice acceptor sequences useful in the invention can be determined from gene sequences using methods known in the art (e.g., Desmet et al., Human Splicing Finder: an online bioinformatics tool to predict splicing signals. Nucleic Acid Research, 2009).

As used herein, the term “C-terminal fragment of a signal peptide” refers to the portion of a signal peptide sequence, which is not encoded by exon 1 of a transferrin gene (i.e., the N-terminal fragment). It is understood that exon 1 of the human and mouse transferrin genes comprise 43 nucleotides (see, SEQ ID NOs: 8 and 16, respectively), wherein nucleotides 1-42 encode 14 amino acids. The nucleotide at position 43 is a “G” which, after splicing, pairs with the “GG” at the 5′ end of exon 2 to encode a glycine. Thus, it is further understood that the first two nucleotides of an exogenous nucleic acid encoding a C-terminal fragment of a signal peptide pair with the nucleotide at position 43 of exon 1 in order to encode an amino acid, and that the remaining nucleotides of the exogenous nucleic acid sequence encode the remaining amino acids of the C-terminal fragment of the signal peptide.

As used herein, the term “exogenous polypeptide of interest” or “polypeptide of interest” refers to a polypeptide that is encoded by a sequence in an exogenous nucleic acid molecule of the invention, and which is not normally encoded by the gene (e.g., a transferrin gene) in which the exogenous nucleic acid molecule is located.

The terms “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. As used herein, a “vector” can also refer to a viral vector. Viral vectors can include, without limitation, retroviral vectors, lentiviral vectors, adenoviral vectors, and adeno-associated viral vectors (AAV).

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the terms “treatment” or “treating a subject” refers to the administration of an engineered nuclease of the invention, or a nucleic acid encoding an engineered nuclease of the invention, in combination with a template including an exogenous nucleic acid molecule encoding a polypeptide of interest to a subject having a disorder (e.g., such as one in Table 3). In some aspects, an engineered nuclease of the invention or a nucleic acid encoding the same is administered during treatment in the form of a pharmaceutical composition of the invention.

The term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In some specific embodiments, an effective amount of the engineered nuclease and/or template nucleic acid (e.g., an engineered meganuclease) comprises about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding the engineered nuclease or of a template nucleic acid. In specific embodiments, an effective amount of a nucleic acid encoding an engineered nuclease and/or a template nucleic acid, or a pharmaceutical composition comprising a nucleic acid encoding an engineered nuclease and/or a template nucleic acid disclosed herein, reduces at least one symptom of a disease in a subject.

The term “gc/kg” or “gene copies/kilogram” refers to the number of copies of a nucleic acid encoding an engineered nuclease or the number of copies of a template nucleic acid described herein per weight in kilograms of a subject that is administered the nucleic acid encoding the engineered nuclease and/or the template nucleic acid.

The term “lipid nanoparticle” refers to a lipid composition having a typically spherical structure with an average diameter between 10 and 1000 nanometers. In some formulations, lipid nanoparticles can comprise at least one cationic lipid, at least one non-cationic lipid, and at least one conjugated lipid. Lipid nanoparticles known in the art that are suitable for encapsulating nucleic acids, such as mRNA, are contemplated for use in the invention.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

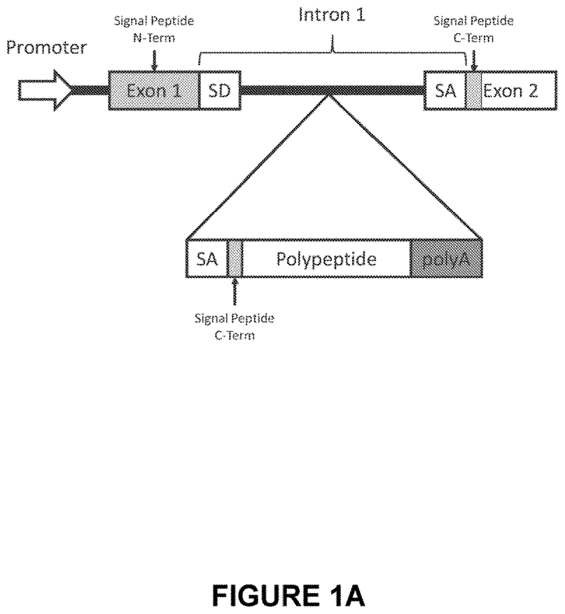

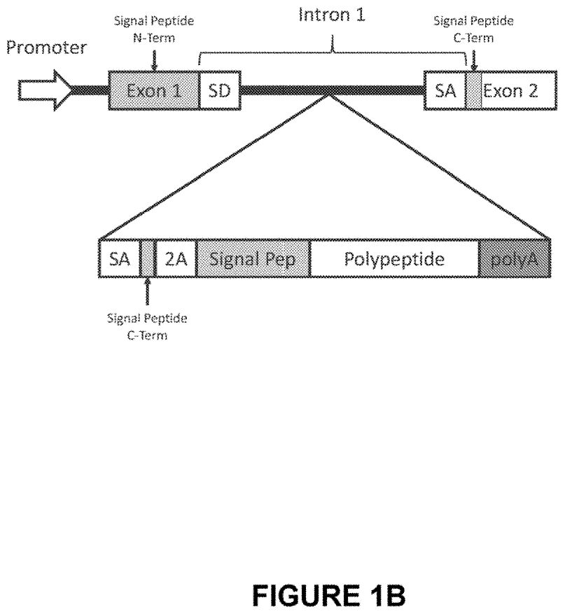

The present invention is based, in part, on the hypothesis that the transferrin gene of a cell can be genetically-modified to express and secrete an exogenous polypeptide under the control of the endogenous transferrin promoter. Specifically, the transferrin gene can be modified by introducing an exogenous nucleic acid molecule into intron 1 at a position between the endogenous splice donor site and the beginning of exon 2. In general, the exogenous nucleic acid molecule comprises a number of elements (see, FIG. 1), arranged 5′ to 3′ as follows: (i) an exogenous splice acceptor sequence, (ii) a first nucleic acid sequence encoding the C-terminal fragment of a signal peptide, such as the C-terminal fragment of the transferrin signal peptide which is encoded by exon 2 of the transferrin gene, (iii) a second nucleic acid sequence encoding the polypeptide of interest, and (iv) a polyA signal.

The exogenous splice acceptor sequence comprises an exogenous branch point sequence and an exogenous splice acceptor site sequence and is capable of joining with the endogenous splice donor site of intron 1. The exogenous splice acceptor sequence operably links the exogenous nucleic acid molecule with the endogenous transferrin promoter. In other words, it allows expression of the exogenous nucleic acid molecule to be driven by the endogenous transferrin promoter. The exogenous splice acceptor sequence can comprise sequences that are identical to, or derived from, sequences found at the 3′ end of intron 1 of a transferrin gene, including the naturally-occurring branch point and splice acceptor site sequences. Alternatively, the exogenous splice acceptor sequence can comprise sequences derived from a gene other than the transferrin gene.

The transferrin gene naturally encodes a signal peptide that allows for protein secretion. The N-terminal fragment of the signal peptide is encoded by exon 1 of the gene, while the C-terminal fragment is encoded by the first 14 base pairs of exon 2. Thus, in order for a complete signal peptide to be produced, the exogenous nucleic acid molecule of the invention includes a first nucleic acid sequence encoding a C-terminal fragment of a signal peptide (e.g., the base pairs from exon 2 of the transferrin gene). Once the endogenous splice donor site and the exogenous splice acceptor sequence join, this approach allows for a complete signal peptide to be encoded and fused to the polypeptide of interest, which can then be secreted from the cell.

The exogenous nucleic acid molecule further comprises a second nucleic acid sequence encoding the polypeptide of interest. This can be any polypeptide of interest, including a secreted polypeptide, which produces a therapeutic effect in a subject suffering from a disease. The exogenous nucleic acid molecule also comprises a polyA signal to terminate translation.

Other elements can also be included in the exogenous nucleic acid molecule to modulate and/or optimize insertion of the construct and expression of the polypeptide of interest. For example, the exogenous nucleic acid molecule can optionally include an IRES sequence or 2A sequence (e.g., T2A, P2A, E2A, or F2A), in combination with an additional nucleic acid sequence encoding a full-length signal peptide. Additionally, the exogenous nucleic acid molecule can optionally include nuclease binding sites.

The approaches described herein have a number of significant advantages for therapeutic treatment. For example, in some embodiments, a secreted polypeptide is produced that does not include any fragment of the endogenous transferrin protein other than the signal peptide. Additionally, in these embodiments, no fragments of the endogenous transferrin protein are produced when the gene is modified as described.

Accordingly, provided herein are engineered nucleases that bind and cleave a recognition sequence within intron 1 of a transferrin gene, such as intron 1 of the human transferrin gene (SEQ ID NO: 4) or intron 1 of the mouse transferrin gene (SEQ ID NO: 12). The present invention also provides methods of using such engineered nucleases to produce a genetically-modified eukaryotic cell comprising a modified transferrin gene. Further provided herein are pharmaceutical compositions and methods for treatment of a variety of conditions (e.g., a disease in Table 3) through expression of a polypeptide of interest (e.g., a polypeptide of interest in Table 3) encoded by an exogenous nucleic acid molecule inserted in intron 1 of a transferrin gene and expressed under the control of the endogenous transferrin promoter.

### 2.2 Nucleases for Recognizing and Cleaving Recognition Sequences within Intron 1 of a Transferrin Gene

It is known in the art that it is possible to use a site-specific nuclease to make a DNA break in the genome of a living cell, and that such a DNA break can result in permanent modification of the genome via homologous recombination with a transgenic DNA sequence. The use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus. Such exogenous nucleic acids can encode any sequence or polypeptide of interest.

Thus, in different embodiments, a variety of different types of nucleases are useful for practicing the invention. In one embodiment, the invention can be practiced using engineered recombinant meganucleases. In another embodiment, the invention can be practiced using a CRIPSR system nuclease or CRISPR system nickase. Methods for making CRIPSR system nucleases and CRISPR system nickases that recognize pre-determined DNA sites are known in the art, for example Ran, et al. (2013) Nat Protoc. 8:2281-308. In another embodiment, the invention can be practiced using TALENs or Compact TALENs. Methods for making TALE domains that bind to pre-determined DNA sites are known in the art, for example Reyon et al. (2012) Nat Biotechnol. 30:460-5. In another embodiment, the invention can be practiced using zinc finger nucleases (ZFNs). In a further embodiment, the invention can be practiced using megaTALs.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site), and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. For example, for an engineered meganuclease of the invention, DNA cleavage can occur between positions 13 and 14 of the sense strand or antisense strand of the 22 base pair recognition sequence.

In some examples, nucleases of the invention have been engineered to bind and cleave a TFN 3-4 recognition sequence (SEQ ID NO: 19). The TFN 3-4 recognition sequence is positioned within intron 1 of the human transferrin gene (SEQ ID NO: 4). An exemplary TFN 3-4 meganuclease (e.g., TFN 3.4x.2) is provided in SEQ ID NO: 23.

In some examples, nucleases of the invention have been engineered to bind and cleave a TFN 19-20 recognition sequence (SEQ ID NO: 21). The TFN 19-20 recognition sequence is positioned within intron 1 of the mouse transferrin gene (SEQ ID NO: 12). An exemplary TFN 19-20 meganuclease (e.g., TFN 19-20x.76) is provided in SEQ ID NO: 26.

Recombinant meganucleases of the invention comprise a first subunit, comprising a first hypervariable (HVR1) region, and a second subunit, comprising a second hypervariable (HVR2) region. Further, the first subunit binds to a first recognition half-site in the recognition sequence (e.g., the TFN3 or TFN19 half-site), and the second subunit binds to a second recognition half-site in the recognition sequence (e.g., the TFN4 or TFN20 half-site). In embodiments where the engineered meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit. An exemplary TFN 3-4 meganuclease of the invention is provided in Table 1. An exemplary TFN 19-20 meganuclease of the invention is provided in Table 2.

TABLE 1
Exemplary meganuclease engineered to bind and cleave the TFN
3-4 recognition sequence (SEQ ID NO: 19).

| | AA | TFN3 | TFN3 | TFN4 | TFN4 |
|---|---|---|---|---|---|
| | SEQ | Subunit | Subunit | Subunit | Subunit |
| Meganuclease | ID | Residues | SEQ ID | Residues | SEQ ID |
| TFN 3-4x.2 | 23 | 198-344 | 24 | 7-153 | 25 |

TABLE 2
Exemplary meganuclease engineered to bind and cleave the
TFN 19-20 recognition sequence (SEQ ID NO: 21).

| | AA | TFN19 | TFN19 | TFN20 | TFN20 |
|---|---|---|---|---|---|
| | SEQ | Subunit | Subunit | Subunit | Subunit |
| Meganuclease | ID | Residues | SEQ ID | Residues | SEQ ID |
| TFN 19-20x.76 | 26 | 198-344 | 27 | 7-153 | 28 |

### 2.3 Templates Encoding Exogenous Nucleic Acids

The present invention further provides a template nucleic acid (e.g., a repair template) that includes an exogenous nucleic acid molecule. By providing the template nucleic acid and a nucleic acid encoding an engineered nuclease of the invention (or an engineered nuclease polypeptide) to a target cell, an exogenous nucleic acid molecule carried by the template nucleic acid can be inserted within intron 1 of the transferrin gene at a cleavage site of the engineered nuclease. Upon insertion, the exogenous nucleic acid molecule becomes operably linked to the endogenous transferrin promoter which, in turn, can drive expression of the exogenous nucleic acid molecule, including expression of a polypeptide of interest encoded therein.

In some embodiments, the exogenous nucleic acid molecule comprises, from 5′ to 3′: (a) an exogenous splice acceptor sequence; (b) a first nucleic acid sequence encoding a C-terminal fragment of a signal peptide; (c) a second nucleic acid sequence encoding a polypeptide of interest (e.g., a polypeptide in Table 3); and (d) a polyA signal. Each of these elements is described in further detail, below.

The exogenous splice acceptor sequence is a nucleotide sequence comprising an exogenous branch point sequence and an exogenous splice acceptor site sequence. In such sequences, the exogenous branch point is 5′ upstream of the exogenous splice acceptor site and enables the exogenous splice acceptor site to join with the endogenous splice donor site in intron 1 of the transferrin gene. The exogenous branch point and the exogenous splice acceptor site can be separated by a number of nucleotides or, alternatively, can be adjacent to one another, as long as they retain their intended function.

The exogenous branch point and/or the exogenous splice acceptor site can be derived from intron 1 of a transferrin gene but be located in a position at which they are not naturally found in the transferrin gene. Alternatively, the exogenous branch point and/or the exogenous splice acceptor site can be derived from a gene other than the transferrin gene. In some embodiments, the exogenous splice acceptor sequence may comprise the branch point (CCCTCAG) and/or splice acceptor site (AG) found in intron 1 of the human transferrin gene. In other embodiments, the exogenous splice acceptor sequence may comprise the branch point (TCCCAG) and/or splice acceptor site (AG) found in intron 1 of the mouse transferrin gene.

In further embodiments, the exogenous splice acceptor sequence may comprise an exogenous branch point derived from the transferrin gene and an exogenous splice acceptor site derived from another gene. Similarly, in other embodiments, the exogenous splice acceptor sequence may comprise an exogenous branch point derived from another gene and an exogenous splice acceptor site derived from the transferrin gene.

In some embodiments, the exogenous splice acceptor sequence has at least 80%, at least 85%, at least 90%, at least 95%, or more, sequence identity to SEQ ID NO: 10. In a particular embodiment, the exogenous splice acceptor sequence comprises SEQ ID NO: 10. In another such embodiment, the exogenous splice acceptor sequence has at least 80%, at least 85%, at least 90%, at least 95%, or more, sequence identity to SEQ ID NO: 18. In a particular embodiment, the exogenous splice acceptor sequence comprises SEQ ID NO: 18.

The exogenous nucleic acid molecule further includes a nucleic acid sequence encoding the C-terminal fragment of a signal peptide, such as the C-terminal fragment of the transferrin signal peptide, which is naturally encoded by exon 2. The C-terminal fragment can include any range of nucleic acids that can be combined with the N-terminal fragment encoded by exon 1 of the transferrin gene to produce a signal peptide that is capable of directing secretion of the encoded polypeptide of interest. In particular embodiments, the C-terminal end can include between 1-14 nucleotides of the transferrin signal peptide that are present in exon 2, as long as the resulting signal peptide retains the ability to facilitate secretion of a polypeptide. In some embodiments, all 14 nucleotides present in exon 2, which encode the C-terminal fragment of the signal peptide are included. In these embodiments, when combined with the N-terminal fragment encoded by exon 1 of the transferrin gene, a functional signal peptide can be generated and fused to the encoded polypeptide of interest, which can then be secreted from the cell.

Accordingly, in some embodiments, the exogenous nucleic acid molecule includes a first nucleic acid encoding the C-terminal fragment of the transferrin signal peptide, wherein the first nucleic acid has at least 80%, at least 85%, at least 90%, at least 95%, or more, sequence identity to SEQ ID NO: 9. In some embodiments, the exogenous nucleic acid molecule includes a first nucleic acid encoding the C-terminal fragment of the transferrin signal peptide, wherein the first nucleic acid has at least 80%, at least 85%, at least 90%, at least 95%, or more, sequence identity to SEQ ID NO: 17.

The exogenous nucleic acid molecule further includes a second nucleic acid sequence encoding a polypeptide of interest. The encoded polypeptide can be any polypeptide of interest. For example, the polypeptide of interest can be acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase. In some embodiments, the second nucleic acid encodes a polypeptide having at least 80%, at least 85%, at least 90%, at least 95%, or more, sequence identity to any one of acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase. In certain embodiments, the polypeptide is one that confers therapeutic benefits to a subject (e.g., a human) having a disease upon expression from the endogenous transferrin promoter. See Table 3 for examples of related therapeutic uses for expressing the indicated polypeptides of interest.

The exogenous nucleic acid additionally includes a polyadenylation signal (polyA signal). The polyA signal is a nucleic acid sequence (e.g., including AAUAAA) within the 3′ UTR that directs binding of a polyadenylation protein complex within the sequence. Various polyadenylation signals are known such as tk polyA (Cole et al., Mol. Cell Biol., 5, 2104-2113, 1985), SV40 late (Schek et al., Mol. Cell Biol. 12, 5386-5393, 1992) and early polyA or BGH polyA (described for example in U.S. Pat. No. 5,122,458). In some embodiments, the polyA signal comprises a nucleic acid sequence having at least 80%, at least 85%, at least 90%, at least 95%, or more, sequence identity to SEQ ID NO: 34. In certain embodiments, the polyA signal comprises a nucleic acid sequence of SEQ ID NO: 34. The polyadenylation signal of SEQ ID NO: 34 may be substituted with other hexanucleotide sequences with homology to AAUAAA as long as they are capable of signaling polyadenylation of mRNAs. Non limiting examples of homologous hexanucleotide sequences include AAAAAA, AUUAAA, AAUAUA, AAUAAU, UAUAAA, AAUUAA, AAUAAG, AGUAAA, GAUAAA, AAUGAA, AAUAGA, AAGAAA, ACUAAA, CAUAAA, AAUCAA, AACAAA, AAUCAA, and AAUAAC.

The exogenous nucleic acid molecule can further include other elements such as IRES or 2A sequences, or nucleic acid sequences encoding full-length signal peptides, as further described herein. For example, the exogenous nucleic acid molecule may include an IRES sequence (SEQ ID NO: 29) or a 2A sequence, such as T2A (SEQ ID NO: 30), P2A (SEQ ID NO: 31), E2A (SEQ ID NO: 32), or F2A (SEQ ID NO: 33). In some such embodiments, the nucleic acid molecule comprises an IRES sequence (SEQ ID NO: 29), a T2A sequence (SEQ ID NO: 30), a P2A sequence (SEQ ID NO: 31), an E2A sequence (SEQ ID NO: 32), or an F2A sequence (SEQ ID NO: 33) positioned 3′ downstream of the exogenous splice acceptor sequence and 5′ upstream of the second nucleic acid sequence encoding a polypeptide of interest.

In embodiments where the exogenous nucleic acid molecule includes an IRES or 2A sequence, a sequence encoding a full-length signal peptide is also included 3′ downstream of the IRES or 2A sequence and 5′ upstream of the second nucleic acid sequence encoding a polypeptide of interest. The full-length signal peptide is included in instances where the IRES or 2A sequence is present since the IRES or 2A sequence creates a break from the signal peptide that is otherwise formed by the endogenous N-terminal signal peptide encoded by exon 1 of the transferrin gene and the C-terminal signal peptide fragment encoded by the first nucleic acid sequence. The nucleic acid sequence encoding the signal peptide can be positioned 3′ downstream of the IRES or 2A sequence and 5′ upstream of the second nucleic acid sequence. The signal peptide can be any signal peptide which enables the polypeptide of interest to be secreted. In some such embodiments, the exogenous nucleic acid molecule comprises a nucleic acid sequence encoding a signal peptide and having at least 80%, at least 85%, at least 90%, at least 95%, or more, sequence identity to SEQ ID NO: 7 (i.e., the human transferrin signal peptide). In certain embodiments, the nucleic acid sequence encoding the signal peptide comprises SEQ ID NO: 7. In other embodiments, the exogenous nucleic acid molecule comprises a nucleic acid sequence encoding a signal peptide and having at least 80%, at least 85%, at least 90%, at least 95%, or more, sequence identity to SEQ ID NO: 15 (i.e., the mouse transferrin signal peptide). In certain embodiments, the nucleic acid sequence encoding the signal peptide comprises SEQ ID NO: 14. In other embodiments, the nucleic acid sequence encoding the signal peptide is not derived from a transferrin gene.

In particular embodiments, where the exogenous nucleic acid molecule includes an IRES or 2A sequence and full-length signal peptide, the exogenous nucleic acid molecule comprises, from 5′ to 3′: (a) an exogenous splice acceptor sequence; (b) a first nucleic acid sequence encoding a C-terminal fragment of a signal peptide; (c) an IRES sequence (SEQ ID NO: 29) or 2A sequence (e.g., T2A (SEQ ID NO: 30), P2A (SEQ ID NO: 31), E2A (SEQ ID NO: 32), or F2A (SEQ ID NO: 33)); (d) a nucleic acid sequence encoding a full-length signal peptide; (e) a second nucleic acid sequence encoding a polypeptide of interest (e.g., a polypeptide in Table 3); and (f) a polyA signal.

In some embodiments, an exogenous nucleic acid molecule of the invention includes homology arms to promote insertion of the sequence via homologous recombination at an engineered nuclease cleavage site within intron 1 of the transferrin gene. Accordingly, in some embodiments, the exogenous nucleic acid molecule further comprises a 5′ homology arm, which is positioned 5′ upstream of the exogenous splice acceptor sequence, and a 3′ homology arm, which is positioned 3′ downstream of the polyA signal, wherein the 5′ homology arm and the 3′ homology arm are homologous to sequences flanking an engineered nuclease cleavage site of interest within intron 1 of a transferrin gene. In specific embodiments, the 5′ and 3′ homology arms have at least 70%, at least 80%, at least 85%, at least 90%, at least 91%, at least 92%, at least 93%, at least 94%, at least 95%, at least 96%, at least 97%, at least 98%, at least 99%, or 100% sequence identity to the corresponding transferrin sequence in the chromosome of the eukaryotic cell. The 5′ and 3′ homology arms can comprise at least 10, at least 20, at least 30, at least 40, at least 50, at least 75, at least 100, at least 150, at least 200, at least 250, at least 500 base pairs, at least 1000 base pairs or any length sufficient to promote recombination of the exogenous nucleic acid molecule into the eukaryotic chromosome.

In some embodiments, the exogenous nucleic acid molecule comprises, from 5′ to 3′: (a) a 5′ homology arm; (b) an exogenous splice acceptor sequence; (c) a first nucleic acid sequence encoding a C-terminal fragment of a signal peptide; (d) a second nucleic acid sequence encoding a polypeptide of interest (e.g., a polypeptide in Table 3); (e) a polyA signal; and (f) a 3′ homology arm.

In other particular embodiments, where the exogenous nucleic acid molecule includes an IRES or 2A sequence and full-length signal peptide, the exogenous nucleic acid molecule comprises, from 5′ to 3′: (a) a 5′ homology arm; (b) an exogenous splice acceptor sequence; (c) a first nucleic acid sequence encoding a C-terminal fragment of a signal peptide; (d) an IRES sequence (SEQ ID NO: 29) or 2A sequence (e.g., T2A (SEQ ID NO: 30), P2A (SEQ ID NO: 31), E2A (SEQ ID NO: 32), or F2A (SEQ ID NO: 33)); (e) a nucleic acid sequence encoding a full-length signal peptide; (f) a second nucleic acid sequence encoding a polypeptide of interest (e.g., a polypeptide in Table 3); (g) a polyA signal; and (h) a 3′ homology arm.

In some alternative embodiments, the exogenous nucleic acid molecule comprises a first engineered nuclease recognition sequence and a second engineered nuclease recognition sequence flanking the nucleic acid sequence encoding a polypeptide of interest. The engineered nuclease recognition sequence can be designed for an engineered meganuclease, a TALEN, a compact TALEN, a megaTAL, a zinc finger nuclease (ZFN), or a CRISPR system nuclease as described herein. The presence of engineered nuclease recognition sequences flanking the nucleic acid sequence encoding a polypeptide of interest allows for the engineered nuclease to cleave and linearize an exogenous nucleic acid molecule for insertion into the genome of a cell (e.g., intron 1 of the transferrin gene within a cell). In addition, the presence of engineered nuclease binding sites that are complementary to a nuclease binding site located within intron 1 of the transferrin gene when cleaved can further promote insertion of the exogenous nucleic acid molecule at the nuclease cleavage site. Such engineered nucleases that generate complementary overhangs include, for example, those that generate 5′ or 3′ overhangs following cleavage of a recognition sequence. In such embodiments, complementary engineered nuclease recognition sequences that are comprised by the exogenous nucleic acid sequence can be designed or oriented such that, when the exogenous nucleic acid sequence is inserted into the genome of a cell, a recognition sequence for the nuclease is not introduced into the genome. This prevents re-introduction of one or more new engineered nuclease recognition sequences, their subsequent cleavage, and loss of the inserted exogenous nucleic acid molecule from the genome of the cell. In the example of an engineered meganuclease recognition sequence, the elimination of the nuclease recognition sequence may be accomplished by inverting the nuclease recognition sequence such that the nuclease recognition sequence comprised by the exogenous nucleic acid molecule is the reverse complement of the 5′ to 3′ nuclease recognition sequence originally present in the genome of the cell. Examples of inverted recognition sequences for the TFN 3-4 and TFN 19-20 recognition sequences are provided as SEQ ID NOs: 58-61.

In some of these embodiments, the exogenous nucleic acid comprises from 5′ to 3′: (a) a first nuclease recognition sequence; (b) an exogenous splice acceptor sequence; (c) a first nucleic acid sequence encoding a C-terminal fragment of a signal peptide; (d) a second nucleic acid sequence encoding a polypeptide of interest (e.g., a polypeptide in Table 3); (e) a polyA signal; and (f) a second nuclease recognition sequence.

In some alternative embodiments, the exogenous nucleic acid comprises from 5′ to 3′: (a) a first nuclease recognition sequence; (b) a 5′ homology arm; (c) an exogenous splice acceptor sequence; (d) a first nucleic acid sequence encoding a C-terminal fragment of a signal peptide; (e) a second nucleic acid sequence encoding a polypeptide of interest (e.g., a polypeptide in Table 3); (f) a polyA signal; (g) a 3′ homology arm; and (h) a second nuclease recognition sequence.

In additional embodiments the exogenous nucleic acid comprises from 5′ to 3′: (a) a first nuclease recognition sequence; (b) an exogenous splice acceptor sequence; (c) a first nucleic acid sequence encoding a C-terminal fragment of a signal peptide; (d) an IRES sequence (SEQ ID NO: 29) or 2A sequence (e.g., T2A (SEQ ID NO: 30), P2A (SEQ ID NO: 31), E2A (SEQ ID NO: 32), or F2A (SEQ ID NO: 33)); (e) a nucleic acid sequence encoding a full-length signal peptide; (f) a second nucleic acid sequence encoding a polypeptide of interest (e.g., a polypeptide in Table 3); (g) a polyA signal; and (h) a second nuclease recognition sequence.

In further embodiments the exogenous nucleic acid comprises from 5′ to 3′: (a) a first nuclease recognition sequence; (b) a 5′ homology arm; (c) an exogenous splice acceptor sequence; (d) a first nucleic acid sequence encoding a C-terminal fragment of a signal peptide; (e) an IRES sequence (SEQ ID NO: 29) or 2A sequence (e.g., T2A (SEQ ID NO: 30), P2A (SEQ ID NO: 31), E2A (SEQ ID NO: 32), or F2A (SEQ ID NO: 33)); (f) a nucleic acid sequence encoding a full-length signal peptide; (g) a second nucleic acid sequence encoding a polypeptide of interest (e.g., a polypeptide in Table 3); (h) a polyA signal; (i) a 3′ homology arm and (j) a second nuclease recognition sequence.

### 2.4 Methods for Producing Genetically-Modified Cells

The invention provides methods for producing genetically-modified cells, both in vitro and in vivo, using engineered nucleases that bind and cleave recognition sequences found within intron 1 of a transferrin gene. Cleavage at such recognition sequences can allow for NHEJ at the cleavage site or insertion of an exogenous sequence via homologous recombination.

The invention further provides methods for treating a disease (e.g., Pompe disease, Fabry disease, Gaucher disease, Hunter syndrome, Marateaux-Lamy syndrome, Marquio A syndrome, lysosomal acid lipase deficiency, alpha-1-antitrypsin deficiency, adenosine deaminase deficiency, or Hurler syndrome) in a subject by administering a pharmaceutical composition comprising a pharmaceutically acceptable carrier and: (a) a nucleic acid encoding the engineered nuclease, or the engineered nuclease polypeptide, and (b) a template nucleic acid encoding an exogenous nucleic acid molecule of the invention, which encodes the polypeptide of interest.

In each case, the invention includes that an engineered nuclease of the invention, or a nucleic acid encoding the engineered nuclease, can be delivered (i.e., introduced) into cells that would typically express transferrin.

Engineered nucleases of the invention can be delivered into a cell in the form of protein or, preferably, as a nucleic acid encoding the engineered nuclease. Such nucleic acid can be DNA (e.g., circular or linearized plasmid DNA or PCR products) or RNA (e.g., mRNA).

For embodiments in which the engineered nuclease coding sequence is delivered in DNA form, it should be operably linked to a promoter to facilitate transcription of the nuclease gene. Mammalian promoters suitable for the invention include constitutive promoters such as the cytomegalovirus early (CMV) promoter (Thomsen et al. (1984), Proc Natl Acad Sci USA. 81(3):659-63) or the SV40 early promoter (Benoist and Chambon (1981), Nature. 290(5804):304-10) as well as inducible promoters such as the tetracycline-inducible promoter (Dingermann et al. (1992), Mol Cell Biol. 12(9):4038-45). An engineered nuclease of the invention can also be operably linked to a synthetic promoter. Synthetic promoters can include, without limitation, the JeT promoter (WO 2002/012514). In specific embodiments, a nucleic acid sequence encoding an engineered nuclease of the invention can be operably linked to a tissue-specific promoter, such as a liver-specific promoter. Examples of liver-specific promoters include, without limitation, the TBG promoter, the human alpha-1 antitrypsin promoter, hybrid liver-specific promoter (hepatic locus control region from ApoE gene (ApoE-HCR) and a liver-specific alpha1-antitrypsin promoter), human thyroxine binding globulin (TBG) promoter, and apolipoprotein A-II promoter.

In specific embodiments, a nucleic acid sequence encoding at least one engineered nuclease is delivered on a recombinant DNA construct or expression cassette. For example, the recombinant DNA construct can comprise an expression cassette (i.e., “cassette”) comprising a promoter and a nucleic acid sequence encoding an engineered nuclease described herein.

In some embodiments, mRNA encoding the engineered nuclease is delivered to a cell because this reduces the likelihood that the gene encoding the engineered nuclease will integrate into the genome of the cell.

Such mRNA encoding an engineered nuclease can be produced using methods known in the art such as in vitro transcription. In some embodiments, the mRNA is 5′ capped using 7-methyl-guanosine, anti-reverse cap analogs (ARCA) (U.S. Pat. No. 7,074,596), CleanCap® analogs such as Cap 1 analogs (Trilink, San Diego, Calif.), or enzymatically capped using vaccinia capping enzyme or similar. In some embodiments, the mRNA may be polyadenylated. The mRNA may contain various 5′ and 3′ untranslated sequence elements to enhance expression the encoded engineered nuclease and/or stability of the mRNA itself. Such elements can include, for example, posttranslational regulatory elements such as a woodchuck hepatitis virus posttranslational regulatory element. The mRNA may contain nucleoside analogs or naturally-occurring nucleosides, such as pseudouridine, 5-methylcytidine, N6-methyladenosine, 5-methyluridine, or 2-thiouridine. Additional nucleoside analogs include, for example, those described in U.S. Pat. No. 8,278,036.

Purified nuclease proteins can be delivered into cells to cleave genomic DNA, which allows for homologous recombination or non-homologous end-joining at the cleavage site with an exogenous nucleic acid molecule encoding a polypeptide of interest as described herein, by a variety of different mechanisms known in the art, including those further detailed herein.

In another particular embodiment, a nucleic acid encoding an endonuclease of the invention is introduced into the cell using a single-stranded DNA template. The single-stranded DNA can further comprise a 5′ and/or a 3′ AAV inverted terminal repeat (ITR) upstream and/or downstream of the sequence encoding the engineered nuclease. The single-stranded DNA can further comprise a 5′ and/or a 3′ homology arm upstream and/or downstream of the sequence encoding the engineered nuclease.

In another particular embodiment, genes encoding an endonuclease of the invention is introduced into a cell using a linearized DNA template. Such linearized DNA templates can be produced by methods known in the art. For example, a plasmid DNA encoding an endonuclease can be digested by one or more restriction enzymes such that the circular plasmid DNA is linearized prior to being introduced into a cell.

Purified engineered nuclease proteins, or nucleic acids encoding engineered nucleases, can be delivered into cells to cleave genomic DNA by a variety of different mechanisms known in the art, including those further detailed herein below. In some embodiments, about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding the engineered nuclease is administered to the subject. In some embodiments, at least about 1×10¹⁰ gc/kg, at least about 1×10¹¹ gc/kg, at least about 1×10¹² gc/kg, at least about 1×10¹³ gc/kg, or at least about 1×10¹⁴ gc/kg of a nucleic acid encoding the engineered nuclease is administered to the subject. In some embodiments, about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg of a nucleic acid encoding the engineered nuclease is administered to the subject. In certain embodiments, about 1×10¹² gc/kg to about 9×10¹³ gc/kg (e.g., about 1×10¹² gc/kg, about 2×10¹² gc/kg, about 3×10¹² gc/kg, about 4×10¹² gc/kg, about 5×10¹² gc/kg, about 6×10¹² gc/kg, about 7×10¹² gc/kg, about 8×10¹² gc/kg, about 9×10¹² gc/kg, about 1×10¹³ gc/kg, about 2×10¹³ gc/kg, about 3×10¹³ gc/kg, about 4×10¹³ gc/kg, about 5×10¹³ gc/kg, about 6×10¹³ gc/kg, about 7×10¹³ gc/kg, about 8×10¹³ gc/kg, or about 9×10¹³ gc/kg) of a nucleic acid encoding the engineered nuclease is administered to the subject.

The target tissue(s) for delivery of engineered nucleases of the invention include, without limitation, cells of the liver, such as a hepatocyte cell, a primary hepatocyte cell, a human hepatocyte, a human primary hepatocyte, a HepG2.2.15 cell, or a HepG2-hNTCP cell. As discussed, nucleases of the invention can be delivered as purified protein or as RNA or DNA encoding the nuclease. In one embodiment, nuclease proteins, or mRNA, or DNA vectors encoding nucleases, are supplied to target cells (e.g., cells in the liver) via injection directly to the target tissue. Alternatively, nuclease protein, mRNA, DNA, or cells expressing nucleases can be delivered systemically via the circulatory system.

In some embodiments, nuclease proteins, DNA/mRNA encoding nucleases, or cells expressing nuclease proteins are formulated for systemic administration, or administration to target tissues, in a pharmaceutically acceptable carrier in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, proteins/RNA/mRNA/cells are typically admixed with a pharmaceutically acceptable carrier. The carrier must, of course, be acceptable in the sense of being compatible with any other ingredients in the formulation and must not be deleterious to the patient. The carrier can be a solid or a liquid, or both, and can be formulated with the compound as a unit-dose formulation.

In some embodiments, the subject is administered a lipid nanoparticle formulation with about 0.1 mg/kg to about 3 mg/kg of mRNA encoding an engineered nuclease. In some embodiments, the subject is administered a lipid nanoparticle formulation with at least about 0.1 mg/kg, at least about 0.25 mg/kg, at least about 0.5 mg/kg, at least about 0.75 mg/kg, at least about 1.0 mg/kg, at least about 1.5 mg/kg, at least about 2.0 mg/kg, at least about 2.5 mg/kg, or at least about 3.0 mg/kg of mRNA encoding an engineered nuclease. In some embodiments, the subject is administered a lipid nanoparticle formulation within about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of mRNA encoding an engineered nuclease.

In some embodiments, the nuclease proteins, or DNA/mRNA encoding the nuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, engineered nucleases, or DNA/mRNA encoding nucleases, are coupled covalently or non-covalently to an antibody that recognizes a specific cell-surface receptor expressed on target cells such that the nuclease protein/DNA/mRNA binds to and is internalized by the target cells. Alternatively, engineered nuclease protein/DNA/mRNA can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

In some embodiments, nuclease proteins, or DNA/mRNA encoding nucleases, are encapsulated within biodegradable hydrogels for injection or implantation within the desired region of the liver (e.g., in proximity to hepatic sinusoidal endothelial cells or hematopoietic endothelial cells, or progenitor cells which differentiate into the same). Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, nuclease proteins, or DNA/mRNA encoding nucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the nuclease proteins or DNA/mRNA encoding the nucleases are encapsulated within liposomes or complexed using cationic lipids (see, e.g., LIPOFECTAMINE™, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, nuclease proteins, or DNA/mRNA encoding nucleases, are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease proteins, or DNA/mRNA encoding nucleases, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease proteins, or DNA/mRNA encoding nucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, nuclease proteins, or DNA/mRNA encoding nucleases, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and drug release.

In some embodiments, genes encoding a nuclease are introduced into a cell using a viral vector. Such vectors are known in the art and include retroviral vectors, lentiviral vectors, adenoviral vectors, and adeno-associated virus (AAV) vectors (reviewed in Vannucci, et al. (2013 New Microbiol. 36:1-22). Recombinant AAV vectors useful in the invention can have any serotype that allows for transduction of the virus into the cell and insertion of the nuclease gene into the cell genome. For example, in some embodiments, recombinant AAV vectors have a serotype of AAV2, AAV6, AAV8, or AAV9. In some embodiments, the viral vectors are injected directly into target tissues. In alternative embodiments, the viral vectors are delivered systemically via the circulatory system. It is known in the art that different AAV vectors tend to localize to different tissues. In liver target tissues, effective transduction of hepatocytes has been shown, for example, with AAV serotypes 2, 8, and 9 (Sands (2011) Methods Mol. Biol. 807:141-157). Accordingly, in some embodiments, the AAV serotype is AAV2. In alternative embodiments, the AAV serotype is AAV6. In other embodiments, the AAV serotype is AAV8. In still other embodiments, the AAV serotype is AAV9. AAV vectors can also be self-complementary such that they do not require second-strand DNA synthesis in the host cell (McCarty, et al. (2001) Gene Ther. 8:1248-54). Nucleic acids delivered by recombinant AAV vectors can include left (5′) and right (3′) inverted terminal repeats.

If the nuclease genes are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a particular embodiment, nuclease genes are operably linked to a promoter that drives gene expression preferentially in the target cells. Examples of liver-specific promoters include, without limitation, the human alpha-1 antitrypsin promoter, hybrid liver-specific promoter (hepatic locus control region from ApoE gene (ApoE-HCR) and a liver-specific alpha1-antitrypsin promoter), human thyroxine binding globulin (TBG) promoter, and apolipoprotein A-II promoter.

Methods and compositions are provided for delivering a nuclease disclosed herein to the liver of a subject. In one embodiment, native hepatocytes, which have been removed from the mammal, can be transduced with a vector encoding the engineered nuclease. Alternatively, native hepatocytes of the subject can be transduced ex vivo with a viral vector, such as a recombinant AAV vector, which encodes the engineered nuclease and/or a molecule that stimulates liver regeneration, such as a hepatotoxin. Preferably the hepatotoxin is uPA and has been modified to inhibit its secretion from the hepatocyte once expressed by the viral vector. In another embodiment, the vector encodes tPA, which can stimulate hepatocyte regeneration de novo. The transduced hepatocytes, which have been removed from the mammal, can then be returned to the mammal where conditions are provided that are conducive to expression of the engineered nuclease. Typically the transduced hepatocytes can be returned to the patient by infusion through the spleen or portal vasculature, and administration may be single or multiple over a period of 1 to 5 or more days.

In an in vivo aspect of the methods of the invention, a retroviral, pseudotype, or recombinant AAV vector is constructed, which encodes the engineered nuclease and is administered to the subject. Administration of a vector encoding the engineered nuclease can occur, for example, with administration of a recombinant AAV vector that encodes a secretion-impaired hepatotoxin, or encodes tPA, which stimulates hepatocyte regeneration without acting as a hepatotoxin.

In various embodiments of the methods described herein, the one or more engineered nucleases, polynucleotides encoding such engineered nucleases, or vectors comprising one or more polynucleotides encoding such engineered nucleases, as described herein, can be administered via any suitable route of administration known in the art. Accordingly, the one or more engineered nucleases, polynucleotides encoding such engineered nucleases, or vectors comprising one or more polynucleotides encoding such engineered nucleases, as described herein may be administered by an administration route comprising intravenous, intramuscular, intraperitoneal, subcutaneous, intrahepatic, transmucosal, transdermal, intraarterial, and sublingual. Other suitable routes of administration of the engineered nucleases, polynucleotides encoding such engineered nucleases, or vectors comprising one or more polynucleotides encoding such engineered nucleases may be readily determined by the treating physician as necessary.

In some embodiments, a therapeutically effective amount of an engineered nuclease described herein is administered to a subject in need thereof. As appropriate, the dosage or dosing frequency of the engineered nuclease may be adjusted over the course of the treatment, based on the judgment of the administering physician. Appropriate doses will depend, among other factors, on the specifics of any AAV vector chosen (e.g., serotype, etc.), on the route of administration, on the subject being treated (i.e., age, weight, sex, and general condition of the subject), and the mode of administration. Thus, the appropriate dosage may vary from patient to patient. An appropriate effective amount can be readily determined by one of skill in the art. Dosage treatment may be a single dose schedule or a multiple dose schedule. Moreover, the subject may be administered as many doses as appropriate. One of skill in the art can readily determine an appropriate number of doses. The dosage may need to be adjusted to take into consideration an alternative route of administration or balance the therapeutic benefit against any side effects.

The invention further provides for the introduction of an exogenous nucleic acid molecule into the cell, such that the exogenous nucleic acid molecule sequence is inserted into intron 1 of the transferrin gene at a nuclease cleavage site. In some embodiments, the exogenous nucleic acid molecule comprises a 5′ homology arm and a 3′ homology arm to promote homologous recombination of the exogenous nucleic acid molecule into the cell genome at the nuclease cleavage site.

Exogenous nucleic acid molecules of the invention may be introduced into the cell by any of the means previously discussed. In a particular embodiment, exogenous nucleic acid molecules are introduced by way of a viral vector, such as a lentivirus, retrovirus, adenovirus, or a recombinant AAV vector. Recombinant AAV vectors useful for introducing an exogenous nucleic acid molecule can have any serotype that allows for transduction of the virus into the cell and insertion of the exogenous nucleic acid molecule sequence into the cell genome. In some embodiments, recombinant AAV vectors have a serotype of AAV2, AAV6, AAV8, or AAV9. The recombinant AAV vectors can also be self-complementary such that they do not require second-strand DNA synthesis in the host cell. Exogenous nucleic acid molecules introduced using a recombinant AAV can be flanked by a 5′ (left) and 3′ (right) inverted terminal repeat.

In another particular embodiment, an exogenous nucleic acid molecule can be introduced into the cell using a single-stranded DNA template. The single-stranded DNA can comprise the exogenous nucleic acid molecule and, in particular embodiments, can comprise 5′ and 3′ homology arms to promote insertion of the nucleic acid sequence into the nuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, genes encoding a nuclease of the invention and/or an exogenous nucleic acid molecule of the invention can be introduced into the cell by transfection with a linearized DNA template. A plasmid DNA encoding an engineered nuclease and/or an exogenous nucleic acid molecule can, for example, be digested by one or more restriction enzymes such that the circular plasmid DNA is linearized prior to transfection into the cell.

When delivered to a cell, an exogenous nucleic acid of the invention can be operably linked to any promoter suitable for expression of the encoded polypeptide in the cell, including those mammalian promoters and inducible promoters previously discussed. An exogenous nucleic acid of the invention can also be operably linked to a synthetic promoter. Synthetic promoters can include, without limitation, the JeT promoter (WO 2002/012514).

### 2.5 Pharmaceutical Compositions

In some embodiments, the invention provides a pharmaceutical composition comprising a pharmaceutically acceptable carrier and: (a) (i) a nucleic acid encoding an engineered nuclease having specificity for a recognition sequence within intron 1 of a transferrin gene, or (ii) an engineered nuclease polypeptide having specificity for a recognition sequence within intron 1 of a transferrin gene; and (b) a template nucleic acid comprising an exogenous nucleic acid molecule, wherein the exogenous nucleic acid molecule comprises, from 5′ to 3′: (i) an exogenous splice acceptor sequence; (ii) a first nucleic acid sequence encoding a C-terminal fragment of a signal peptide; (iii) a second nucleic acid sequence encoding a polypeptide of interest; and (iv) a polyA signal.

In other embodiments, the invention provides a pharmaceutical composition comprising a pharmaceutically acceptable carrier and a genetically-modified cell of the invention, which can be delivered to a target tissue where the cell can then differentiate into a cell that expresses a modified transferrin gene, e.g., modified to express an exogenous nucleic acid.

In particular embodiments, the pharmaceutical compositions disclosed herein comprise only one population of lipid nanoparticles comprising a mRNA molecule encoding an engineered nuclease or, in other embodiments only one population of recombinant viral vectors (e.g., recombinant AAV vectors) comprising a nucleic acid encoding an engineered nuclease. Such embodiments, using only one population of lipid nanoparticles or viral vectors comprising a nucleic acid molecule encoding the engineered nuclease, can be distinguished from methods that require two or more populations of lipid nanoparticles or viral vectors to deliver unique nucleic acids encoding unique proteins and/or nucleic acids which, when expressed together in a cell, act in concert to cleave a nuclease recognition sequence. For example, engineered TALEN nucleases and engineered zinc finger nucleases would typically require a plurality of populations of lipid nanoparticles or viral vectors, each comprising a different nucleic acid molecule encoding a different part of the functional engineered nuclease (i.e., a left TALEN and a right TALEN, or a left zinc finger nuclease and a right zinc finger nuclease).

Pharmaceutical compositions of the invention can be useful for treating a subject having a disease, such as a disease shown in Table 3, by modifying transferrin in accordance with the present invention to express a polypeptide of interest (e.g., a polypeptide that can confer therapeutic benefits upon expression in the subject having the disease).

TABLE 3

| | | NCBI |
|---|---|---|
| Disease | Polypeptide of Interest | Gene No. |
| Pompe Disease | Acid alpha-glucosidase (GAA) | 2548 |
| Fabry Disease | Alpha-galactosidase | 2717 |
| Gaucher Disease | Glucosylceramidase beta | 2629 |
| Hunter Syndrome | Iduronate-2-sulfatase | 3423 |
| Marateaux-Lamy Syndrome | Arylsulfatase B | 411 |
| Marquio A Syndrome | N-acetylgalactosamine-6- | 2588 |
| | sulfatase | |
| LAL Deficiency | Lysosomal | 3988 |
| | acid lipase | |
| Alpha-1-Antitryp sin | Alpha-1-antitrypsin | 5265 |
| Deficiency | | |
| ADA Deficiency | Adenosine deaminase | 100 |
| Hurler Syndrome | Alpha-L-iduronidase | 3425 |

Such pharmaceutical compositions can be prepared in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, nuclease polypeptides (or DNA/RNA encoding the same or cells expressing the same) are typically admixed with a pharmaceutically acceptable carrier, and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents or biological molecules useful in the treatment of a disease in the subject. Likewise, the additional agent(s) and/or biological molecule(s) can be co-administered as a separate composition.

The pharmaceutical compositions described herein can include any engineered nuclease, or a nucleic acid encoding an engineered nuclease of the invention. The pharmaceutical compositions described herein can further include a template nucleic acid of the invention. In some embodiments, the pharmaceutical composition comprises about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a nucleic acid encoding an engineered nuclease and/or of a template nucleic acid. In some embodiments, the pharmaceutical composition comprises at least about 1×10¹⁰ gc/kg, at least about 1×10¹¹ gc/kg, at least about 1×10¹² gc/kg, at least about 1×10¹³ gc/kg, or at least about 1×10¹⁴ gc/kg of a nucleic acid encoding an engineered nuclease and/or of a template nucleic acid. In some embodiments, the pharmaceutical composition comprises about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg of a nucleic acid encoding an engineered nuclease and/or of a template nucleic acid. In certain embodiments, the pharmaceutical composition comprises about 1×10¹² gc/kg to about 9×10¹³ gc/kg (e.g., about 1×10¹² gc/kg, about 2×10¹² gc/kg, about 3×10¹² gc/kg, about 4×10¹² gc/kg, about 5×10¹² gc/kg, about 6×10¹² gc/kg, about 7×10¹² gc/kg, about 8×10¹² gc/kg, about 9×10¹² gc/kg, about 1×10¹³ gc/kg, about 2×10¹³ gc/kg, about 3×10¹³ gc/kg, about 4×10¹³ gc/kg, about 5×10¹³ gc/kg, about 6×10¹³ gc/kg, about 7×10¹³ gc/kg, about 8×10¹³ gc/kg, or about 9×10¹³ gc/kg) of a nucleic acid encoding an engineered nuclease and/or of a template nucleic acid.

In particular embodiments of the invention, the pharmaceutical composition comprise one or more mRNAs described herein encapsulated within lipid nanoparticles, which are described elsewhere herein.

Some lipid nanoparticles contemplated for use in the invention comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino)butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid comprises from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid comprises from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain particular embodiments, the phospholipid is DPPC, DSPC, or mixtures thereof.

In some embodiments, the non-cationic lipid (e.g., one or more phospholipids and/or cholesterol) comprises from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-di lauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-ω-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition comprises amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are charged more and all anionic charge carriers lose their charge.

Cationic compounds useful for amphoteric liposomes include those cationic compounds previously described herein above. Without limitation, strongly cationic compounds can include, for example: DC-Choi 3-β-[N—(N′,N′-dimethylmethane) carbamoyl] cholesterol, TC-Choi 3-β-[N—(N′, N′, N′-trimethylaminoethane) carbamoyl cholesterol, BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylarnide, DOTMA (1,2-dioleoyloxypropyl)-N,N,N-trimethylamronium chloride) (Lipofectin®), DORIE 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18)2Gly+ N,N-dioctadecylamido-glycine, CTAB cetyltrimethylarnmonium bromide, CpyC cetylpyridinium chloride, DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Some particular examples are PEG-modified diacylglycerols and dialkylglycerols.

In some embodiments, the neutral lipids comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

In some embodiments, the lipid nanoparticles have a composition that specifically enhances delivery and uptake in the liver, or specifically within hepatocytes.

### 2.6 Methods for Producing Recombinant Viral Vectors

In some embodiments, the invention provides viral vectors (e.g., recombinant AAV vectors) for use in the methods of the invention. Recombinant AAV vectors are typically produced in mammalian cell lines such as HEK-293. Because the viral cap and rep genes are removed from the vector to prevent its self-replication to make room for the therapeutic gene(s) to be delivered (e.g. the nuclease gene), it is necessary to provide these in trans in the packaging cell line. In addition, it is necessary to provide the “helper” (e.g. adenoviral) components necessary to support replication (Cots et al. (2013), Curr. Gene Ther. 13(5): 370-81). Frequently, recombinant AAV vectors are produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient.

Because recombinant AAV particles are typically produced (manufactured) in cells, precautions must be taken in practicing the current invention to ensure that the engineered nuclease is not expressed in the packaging cells. Because the viral genomes of the invention may comprise a recognition sequence for the nuclease, any nuclease expressed in the packaging cell line may be capable of cleaving the viral genome before it can be packaged into viral particles. This will result in reduced packaging efficiency and/or the packaging of fragmented genomes. Several approaches can be used to prevent nuclease expression in the packaging cells.

The nuclease can be placed under the control of a tissue-specific promoter that is not active in the packaging cells. For example, if a viral vector is developed for delivery of a nuclease gene(s) to muscle tissue, a muscle-specific promoter can be used. Examples of muscle-specific promoters include C5-12 (Liu, et al. (2004) Hum Gene Ther. 15:783-92), the muscle-specific creatine kinase (MCK) promoter (Yuasa, et al. (2002) Gene Ther. 9:1576-88), or the smooth muscle 22 (SM22) promoter (Haase, et al. (2013) BMC Biotechnol. 13:49-54). Examples of CNS (neuron)-specific promoters include the NSE, Synapsin, and MeCP2 promoters (Lentz, et al. (2012) Neurobiol Dis. 48:179-88). Examples of liver-specific promoters include, for example, albumin promoters (such as Palb), human α1-antitrypsin (such as Pa1AT), and hemopexin (such as Phpx) (Kramer et al., (2003) Mol. Therapy 7:375-85), hybrid liver-specific promoter (hepatic locus control region from ApoE gene (ApoE-HCR) and a liver-specific alpha1-antitrypsin promoter), human thyroxine binding globulin (TBG) promoter, and apolipoprotein A-II promoter. Examples of eye-specific promoters include opsin, and corneal epithelium-specific K12 promoters (Martin et al. (2002) Methods (28): 267-75) (Tong et al., (2007) J Gene Med, 9:956-66). These promoters, or other tissue-specific promoters known in the art, are not highly-active in HEK-293 cells and, thus, will not be expected to yield significant levels of nuclease gene expression in packaging cells when incorporated into viral vectors of the present invention. Similarly, the viral vectors of the present invention contemplate the use of other cell lines with the use of incompatible tissue specific promoters (i.e., the well-known HeLa cell line (human epithelial cell) and using the liver-specific hemopexin promoter). Other examples of tissue specific promoters include: synovial sarcomas PDZD4 (cerebellum), C6 (liver), ASB5 (muscle), PPP1R12B (heart), SLC5A12 (kidney), cholesterol regulation APOM (liver), ADPRHL1 (heart), and monogenic malformation syndromes TP73L (muscle). (Jacox et al., (2010), PLoS One v.5(8):e12274).

Alternatively, the vector can be packaged in cells from a different species in which the nuclease is not likely to be expressed. For example, viral particles can be produced in microbial, insect, or plant cells using mammalian promoters, such as the well-known cytomegalovirus- or SV40 virus-early promoters, which are not active in the non-mammalian packaging cells. In a particular embodiment, viral particles are produced in insect cells using the baculovirus system as described by Gao, et al. (Gao et al. (2007), J. Biotechnol. 131(2):138-43). A nuclease under the control of a mammalian promoter is unlikely to be expressed in these cells (Airenne et al. (2013), Mol. Ther. 21(4):739-49). Moreover, insect cells utilize different mRNA splicing motifs than mammalian cells. Thus, it is possible to incorporate a mammalian intron, such as the human growth hormone (HGH) intron or the SV40 large T antigen intron, into the coding sequence of a nuclease. Because these introns are not spliced efficiently from pre-mRNA transcripts in insect cells, insect cells will not express a functional nuclease and will package the full-length genome. In contrast, mammalian cells to which the resulting recombinant AAV particles are delivered will properly splice the pre-mRNA and will express functional nuclease protein. Haifeng Chen has reported the use of the HGH and SV40 large T antigen introns to attenuate expression of the toxic proteins barnase and diphtheria toxin fragment A in insect packaging cells, enabling the production of recombinant AAV vectors carrying these toxin genes (Chen, H (2012) Mol Ther Nucleic Acids. 1(11): e57).

The nuclease gene can be operably linked to an inducible promoter such that a small-molecule inducer is required for nuclease expression. Examples of inducible promoters include the Tet-On system (Clontech; Chen et al. (2015), BMC Biotechnol. 15(1):4)) and the RheoSwitch system (Intrexon; Sowa et al. (2011), Spine, 36(10): E623-8). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). Practicing the current invention using such ligand-inducible transcription activators includes: 1) placing the nuclease gene under the control of a promoter that responds to the corresponding transcription factor, the nuclease gene having (a) binding site(s) for the transcription factor; and 2) including the gene encoding the transcription factor in the packaged viral genome. The latter step is necessary because the nuclease will not be expressed in the target cells or tissues following recombinant AAV delivery if the transcription activator is not also provided to the same cells. The transcription activator then induces nuclease gene expression only in cells or tissues that are treated with the cognate small-molecule activator. This approach is advantageous because it enables nuclease gene expression to be regulated in a spatio-temporal manner by selecting when and to which tissues the small-molecule inducer is delivered. However, the requirement to include the inducer in the viral genome, which has significantly limited carrying capacity, creates a drawback to this approach.

In another particular embodiment, recombinant AAV particles are produced in a mammalian cell line that expresses a transcription repressor that prevents expression of the nuclease. Transcription repressors are known in the art and include the Tet-Repressor, the Lac-Repressor, the Cro repressor, and the Lambda-repressor. Many nuclear hormone receptors such as the ecdysone receptor also act as transcription repressors in the absence of their cognate hormone ligand. To practice the current invention, packaging cells are transfected/transduced with a vector encoding a transcription repressor and the nuclease gene in the viral genome (packaging vector) is operably linked to a promoter that is modified to comprise binding sites for the repressor such that the repressor silences the promoter. The gene encoding the transcription repressor can be placed in a variety of positions. It can be encoded on a separate vector; it can be incorporated into the packaging vector outside of the ITR sequences; it can be incorporated into the cap/rep vector or the adenoviral helper vector; or it can be stably integrated into the genome of the packaging cell such that it is expressed constitutively. Methods to modify common mammalian promoters to incorporate transcription repressor sites are known in the art. For example, Chang and Roninson modified the strong, constitutive CMV and RSV promoters to comprise operators for the Lac repressor and showed that gene expression from the modified promoters was greatly attenuated in cells expressing the repressor (Chang and Roninson (1996), Gene 183:137-42). The use of a non-human transcription repressor ensures that transcription of the nuclease gene will be repressed only in the packaging cells expressing the repressor and not in target cells or tissues transduced with the resulting recombinant AAV vector.

### 2.7 Engineered Nuclease Variants

Embodiments of the invention encompass the engineered nucleases described herein, and variants thereof. Further embodiments of the invention encompass polynucleotides comprising a nucleic acid sequence encoding the nucleases described herein, template nucleic acids described herein, the exogenous nucleic acid molecules described herein, and variants thereof.

As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein; for example, the ability to bind and cleave recognition sequences found in intron 1 of the transferrin gene, such as the TFN 3-4 recognition sequence (SEQ ID NO: 19) or the TFN 19-20 recognition sequence (SEQ ID NO: 21). Such variants may result, for example, from human manipulation. In some embodiments, biologically active variants of a native polypeptide of the embodiments (e.g., SEQ ID NOs: 23 or 26), or biologically active variants of the recognition half-site binding subunits described herein, will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, native subunit, native HVR1, or native HVR2 as determined by sequence alignment programs and parameters described elsewhere herein. A biologically active variant of a polypeptide or subunit of the embodiments may differ from that polypeptide or subunit by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some embodiments, engineered meganucleases of the invention can comprise variants of the HVR1 and HVR2 regions disclosed herein. Parental HVR regions can comprise, for example, residues 24-79 or residues 215-270 of the exemplified engineered meganucleases. Thus, variant HVRs can comprise an amino acid sequence having at least 80%, at least 85%, at least 90%, at least 91%, at least 92%, at least 93%, at least 94%, at least 95%, at least 96%, at least 97%, at least 98%, at least 99%, or more, sequence identity to an amino acid sequence corresponding to residues 24-79 or residues 215-270 of the engineered meganucleases exemplified herein, such that the variant HVR regions maintain the biological activity of the engineered meganuclease (i.e., binding to and cleaving the recognition sequence). Further, in some embodiments of the invention, a variant HVR1 region or variant HVR2 region can comprise residues corresponding to the amino acid residues found at specific positions within the parental HVR. In this context, “corresponding to” means that an amino acid residue in the variant HVR is the same amino acid residue (i.e., a separate identical residue) present in the parental HVR sequence in the same relative position (i.e., in relation to the remaining amino acids in the parent sequence). By way of example, if a parental HVR sequence comprises a serine residue at position 26, a variant HVR that “comprises a residue corresponding to” residue 26 will also comprise a serine at a position that is relative (i.e., corresponding) to parental position 26.

In particular embodiments, engineered meganucleases of the invention comprise an HVR1 that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 215-270 of SEQ ID NOs: 23 or 26.

In certain embodiments, engineered meganucleases of the invention comprise an HVR2 that has 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 24-79 of SEQ ID NOs: 23 or 26.

A substantial number of amino acid modifications to the DNA recognition domain of the wild-type I-CreI meganuclease have previously been identified (e.g., U.S. Pat. No. 8,021,867) which, singly or in combination, result in engineered meganucleases with specificities altered at individual bases within the DNA recognition sequence half-site, such that the resulting rationally-designed meganucleases have half-site specificities different from the wild-type enzyme. Table 4 provides potential substitutions that can be made in a engineered meganuclease monomer or subunit to enhance specificity based on the base present at each half-site position (−1 through −9) of a recognition half-site.

TABLE 2

| | Favored Sense-Strand Base | | | | | | | | | | |
|---|---|---|---|---|---|---|---|---|---|---|---|
| | | | | | | | | | | A/G/ | A/C/G/ |
| Posn. | A | C | G | T | A/T | A/C | A/G | C/T | G/T | T | T |
| −1 | | R70 | | | | | | | | | |
| | Y75 | * | K70 | Q70* | | | | T46* | | | G70 |
| | | H75 | | | | | | | | | |
| | L75* | * | E70* | C70 | | | | | | | A70 |
| | C75* | R75* | E75* | L70 | | | | | | | S70 |
| | Y139 | H46 | | | | | | | | | |
| | * | * | E46* | Y75* | | | | | | | G46* |
| | | K46 | D46 | | | | | | | | |
| | C46* | * | * | Q75* | | | | | | | |
| | A46* | R46* | | H75* | | | | | | | |
| | | | | H139 | | | | | | | |
| | | | | Q46* | | | | | | | |
| | | | | H46* | | | | | | | |
| −2 | | | | Q44 | | | | | | | |
| | Q70 | E70 | H70 | * | C44* | | | | | | |
| | | | D44 | | | | | | | | |
| | T44* | D70 | * | | | | | | | | |
| | | K44 | | | | | | | | | |
| | A44* | * | E44* | | | | | | | | |
| | V44* | R44* | | | | | | | | | |
| | I44* | | | | | | | | | | |
| | L44* | | | | | | | | | | |
| | N44* | | | | | | | | | | |
| −3 | | | | | | | | | K6 | | |
| | Q68 | E68 | R68 | M68 | | H68 | | Y68 | 8 | | |
| | C24* | F68 | | C68 | | | | | | | |
| | | K24 | | | | | | | | | |
| | I24* | * | L68 | | | | | | | | |
| | | R24* | | F68 | | | | | | | |
| −4 | A26* | E77 | R77 | | | | | S77 | | | S26* |
| | | K26 | | | | | | Q26 | | | |
| | Q77 | * | E26* | | | | | * | | | |
| −5 | | | | | | K28 | C28 | | | | |
| | | E42 | R42 | | | * | * | | | | M66 |
| | | | | | | | Q42 | | | | K66 |
| −6 | Q40 | E40 | R40 | C40 | A40 | | | | | | S40 |
| | C28* | R28* | | I40 | A79 | | | | | | S28* |
| | | | | | A28 | | | | | | |
| | | | | V40 | * | | | | | | |
| | | | | | H28 | | | | | | |
| | | | | C79 | * | | | | | | |
| | | | | I79 | | | | | | | |
| | | | | V79 | | | | | | | |
| | | | | Q28* | | | | | | | |
| −7 | N30* | E38 | K38 | I38 | | | C38 | | | | H38 |
| | | K30 | | | | | | | | | |
| | Q38 | * | R38 | L38 | | | | | | | N38 |
| | | R30* | E30* | | | | | | | | Q30* |
| −8 | F33 | E33 | F33 | L33 | | R32* | R33 | | | | |
| | Y33 | D33 | H33 | V33 | | | | | | | |
| | | | | I33 | | | | | | | |
| | | | | F33 | | | | | | | |
| | | | | C33 | | | | | | | |
| −9 | | E32 | R32 | L32 | | | | D32 | | | S32 |
| | | | K32 | V32 | | | | I32 | | | N32 |
| | | | | A32 | | | | | | | H32 |
| | | | | C32 | | | | | | | Q32 |
| | | | | | | | | | | | T32 |

Bold entries are wild-type contact residues and do not constitute “modifications” as used herein.
An asterisk indicates that the residue contacts the base on the antisense strand.

Certain modifications can be made in an engineered meganuclease monomer or subunit to modulate DNA-binding affinity and/or activity. For example, an engineered meganuclease monomer or subunit described herein can comprise a G, S, or A at a residue corresponding to position 19 of I-CreI or SEQ ID NOs: 23 or 26 (WO 2009001159), a Y, R, K, or D at a residue corresponding to position 66 of I-CreI or SEQ ID NOs: 23 or 26 and/or an E, Q, or K at a residue corresponding to position 80 of I-CreI or SEQ ID NOs: 23 or 26 (U.S. Pat. No. 8,021,867).

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode an engineered nuclease, or an exogenous nucleic acid molecule, or template nucleic acid of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its ability to preferentially bind and cleave recognition sequences found within intron 1 of a transferrin gene. (e.g., exon 1 of the human transferrin gene; SEQ ID NO: 4).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Meganucleases that Bind and Cleave TFN Recognition Sequences

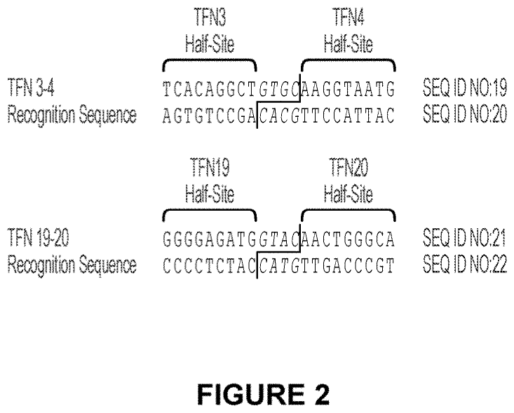

The TFN 3-4 meganuclease described herein (SEQ ID NO: 23) was engineered to bind and cleave the TFN 3-4 recognition sequence (SEQ ID NO: 19), which is present within intron 1 of the human transferrin gene. This meganuclease comprises an N-terminal nuclease-localization signal derived from SV40, a first meganuclease subunit, a linker sequence, and a second meganuclease subunit. A first subunit in the TFN 3-4 meganuclease binds to the TFN3 recognition half-site of SEQ ID NO: 19, while a second subunit binds to the TFN4 recognition half-site (see, FIG. 2). The TFN3-binding subunit and TFN4-binding subunit each comprises a 56 base pair hypervariable region, referred to as HVR1 and HVR2, respectively.

The HVR1 region of the TFN3-binding subunit consists of residues 215-270 of SEQ ID NO: 23. The HVR2 region of the TFN4-binding subunit consists of residues 24-79 of SEQ ID NO: 23.

The TFN 19-20 meganuclease described herein (SEQ ID NO: 26) was engineered to bind and cleave the TFN 19-20 recognition sequence (SEQ ID NO: 21), which is present within intron 1 of the mouse transferrin gene. This meganuclease comprises an N-terminal nuclease-localization signal derived from SV40, a first meganuclease subunit, a linker sequence, and a second meganuclease subunit. A first subunit in the TFN 19-20 meganuclease binds to the TFN19 recognition half-site of SEQ ID NO: 21, while a second subunit binds to the TFN20 recognition half-site (see, FIG. 2). The TFN19-binding subunit and TFN20-binding subunit each comprises a 56 base pair hypervariable region, referred to as HVR1 and HVR2, respectively.

The HVR1 region of the TFN19-binding subunit consists of residues 215-270 of SEQ ID NO: 26. The HVR2 region of the TFN20-binding subunit consists of residues 24-79 of SEQ ID NO: 26.

### Example 2

### Characterization of Meganucleases Having Specificity for the TFN 3-4 or TFN 19-20 Recognition Sequence

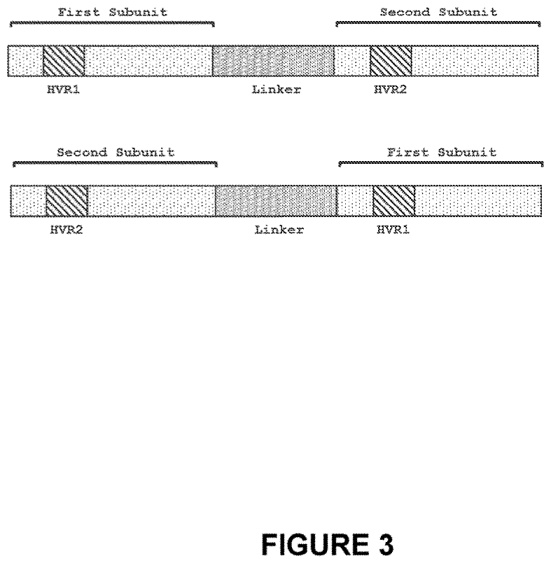

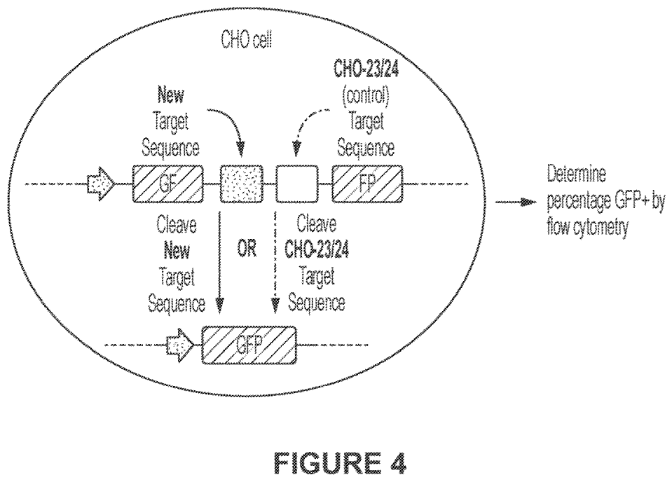

To determine whether TFN meganucleases could bind and cleave the human TFN 3-4 (SEQ ID NO: 19) or murine TFN 19-20 (SEQ ID NO: 21) recognition sequences, the TFN 3-4x.2 (SEQ ID NO: 23) and TFN 19-20x.76 (SEQ ID NO: 26) meganucleases were evaluated using the CHO cell reporter assay previously described (see WO/2012/167192, FIG. 3). To perform the assay, a pair of CHO cell reporter lines were produced that carried a nonfunctional Green Fluorescent Protein (GFP) gene expression cassette integrated into the genome of the cell. The GFP gene in each cell line was interrupted by a pair of recognition sequences such that intracellular cleavage of either recognition sequence by a meganuclease would stimulate a homologous recombination event resulting in a functional GFP gene. In both cell lines, one of the recognition sequences was derived from TFN 3-4 or TFN 19-20, and the second recognition sequence was specifically recognized by a control meganuclease called “CHO 23/24”. CHO reporter cells comprising the TFN 3-4 recognition sequence (SEQ ID NO: 19) and the CHO 23/24 recognition sequence are referred to herein as “TFN 3-4 cells.” CHO reporter cells comprising the TFN 19-20 recognition sequence (SEQ ID NO: 21) and the CHO 23/24 recognition sequence are referred to herein as “TFN 19-20 cells.”

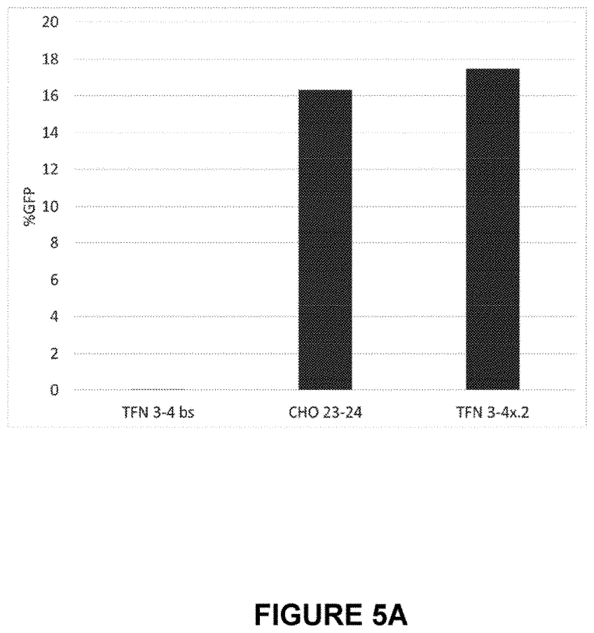

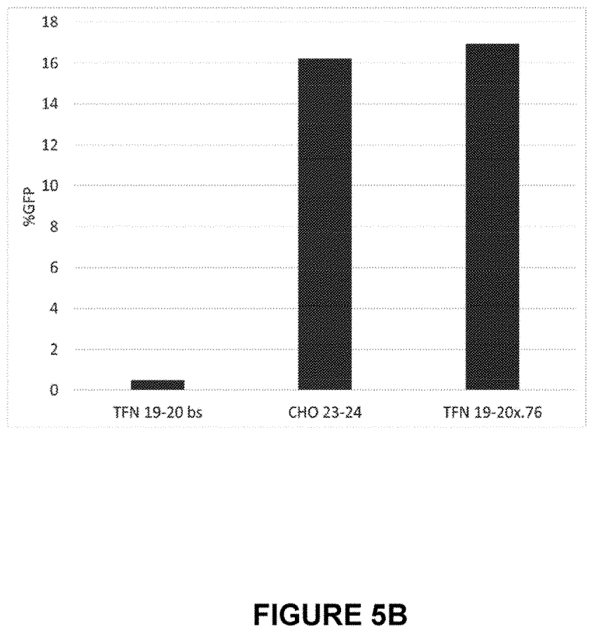

TFN 3-4 or TFN 19-20 cells were transfected with plasmid DNA encoding the TFN 3-4x.2 or TFN 19-20x.76 meganuclease, respectively, or encoding the CHO 23/34 meganuclease. 4×10⁵ CHO cells were transfected with 50 ng of plasmid DNA in a 96-well plate using Lipofectamine 2000 (ThermoFisher) according to the manufacturer's instructions. At 48 hours post-transfection, cells were evaluated by flow cytometry to determine the percentage of GFP-positive cells compared to an untransfected negative control (1-2 bs). Each TFN meganuclease was found to produce GFP-positive cells in cell lines comprising its respective recognition sequence at frequencies significantly exceeding the negative control and comparable to or exceeding the CHO 23/24 positive control, indicating that each TFN meganuclease was able to efficiently bind and cleave the intended TFN recognition sequence in a cell (FIG. 5A and FIG. 5B).

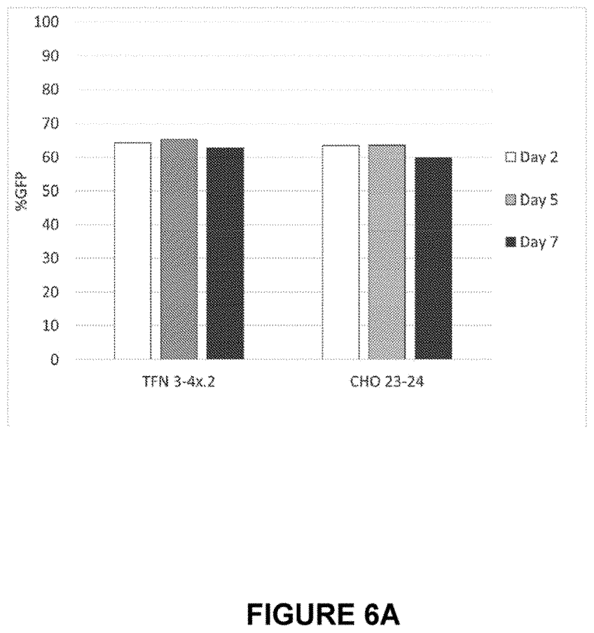

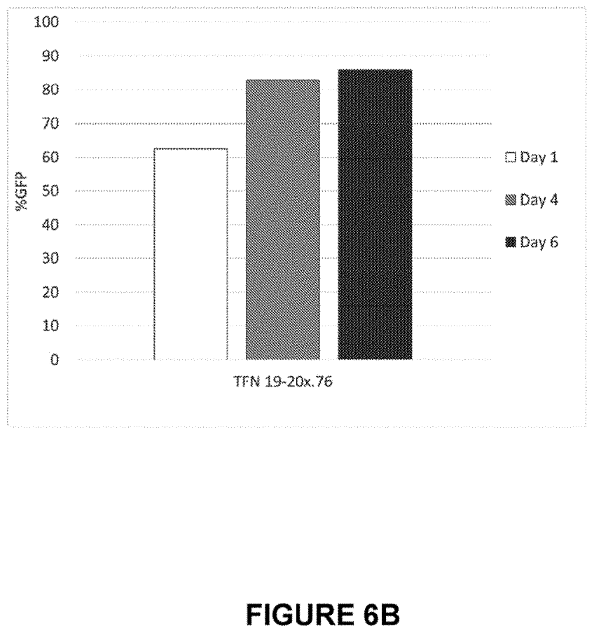

The efficacy of each TFN meganuclease was also determined in a time-dependent manner between 1 and 7 days after introduction of the meganucleases mRNA into TFN 3-4 or TFN 19-20 cells. In this study, TFN 3-4 or TFN 19-20 cells (1.0×10⁶) were electroporated with 1×10⁶ copies of meganuclease mRNA per cell using a BioRad Gene Pulser Xcell according to the manufacturer's instructions. At 48 hours post-transfection, cells were evaluated by flow cytometry to determine the percentage of GFP-positive cells. A CHO 23/24 meganuclease was also included at each time point as a positive control. Each of the meganucleases showed a comparable GFP-positive percentage relative to CHO 23-24 that was stable or increasing over time (FIGS. 6A and 6B). These results demonstrate the ability of these TFN meganucleases to bind and cleave their respective recognition sequence in the genome of a cell.

### Example 3

### In Vitro Experiments Demonstrating Insertion of a Reporter Construct into Transferrin Intron 1 and Expression of the Inserted Transgene Using the Endogenous Transferrin Promoter

### A. Method

Molecular Cloning

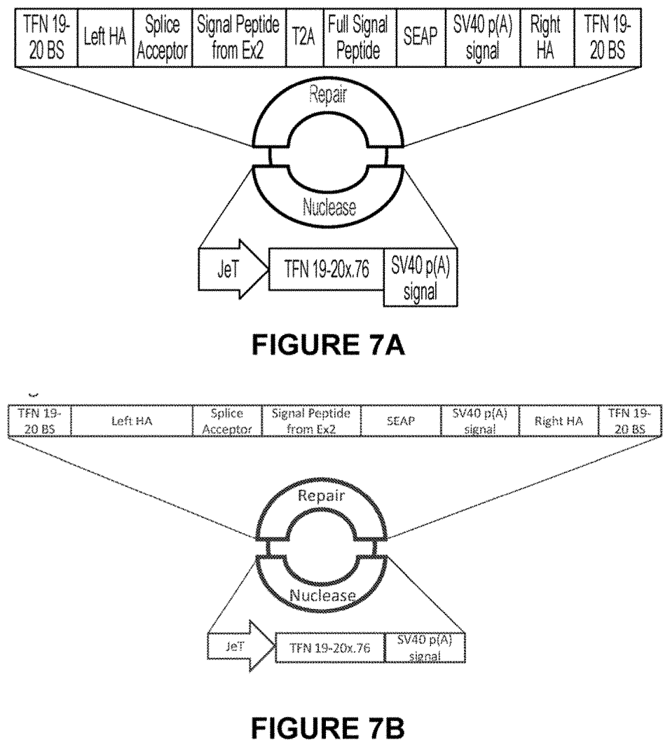

SEAP repair constructs were cloned using standard molecular biology cloning techniques. Constructs included ˜500 bp homology arms flanking a repair cassette containing the splice acceptor that precedes the endogenous transferrin exon 2, the remaining bases of the signal peptide in transferrin exon 2, and the SEAP transgene with or without a T2A ribosome-skipping peptide sequence (FIG. 7). In constructs where the T2A sequence is present, the SEAP transgene has been fused to the transferrin signal peptide and the SEAP signal peptide has been removed. In all constructs, the entire repair construct is flanked by recognition sites for the TFN 19-20x.76 nuclease. Plasmids also comprise a nuclease cassette encoding the TFN 19-20x.76 nuclease driven by a JeT promoter or a truncated TFN 19-20x.76 (truncTFN 19-20) nuclease to serve as a control. A repair cassette containing the T2A sequence is provided in SEQ ID NO: 51 and a repair cassette without the T2A sequence is provided in SEQ ID NO: 52.

Cell Culture and Transfection

FL83B (ATCC CRL-2390) cells were electroporated using the Lonza 4D Amaxa Nucleofector system using 1×10⁶ cells and 1.5 ug plasmid DNA. Cells were seeded in 6 well plates in 2 ml complete growth media. Conditioned media was collected and centrifuged at 3000 g for 5 minutes at 3 and 10 days post-electroporation. Transfection efficiency was measured 24 hours post-electroporation using a GFP transfected positive control and flow cytometry (Beckman CytoFlex S).

SEAP Assay

Conditioned media was assayed for SEAP expression using the Phospha light SEAP Reporter Gene Assay system (Thermo) according to the manufacturer's protocol. Media was diluted 1:3 and chemiluminescence was measured using a Molecular Devices Spectramax i3 plate reader.

PCR for Transgene Insertion

Genomic DNA was isolated from cells (Machery Nagel QuickPure Blood Kit). PCR (NEB Q5 Hot Start DNA Polymerase) with P1 and P2 was conducted and imaged on a 1% agarose gel to identify integrated transgenes. A product of 1113 bases (for Repair TFN SEAP) or 1233 bases (for Repair TFN T2A SEAP) correlates with insertion of the transgene by homology-directed repair (HDR).

P1: cgtggatccacatgcatctggg (SEQ ID NO: 36)

P2: cccagtgcctctgcagcttc (SEQ ID NO: 37)

### B. Results

To test whether the endogenous transferrin promoter could be used to drive transgene expression, a number of repair donor plasmids were designed (FIG. 7). Each repair plasmid included a repair cassette with homology arms to the transferrin sequence, the splice acceptor that precedes the transferrin exon 2, and the bases that encode transferrin signal peptide from exon 2. Additionally, each repair plasmid contained two target sequences for the TFN 19-20 nuclease just outside the homology arms. This allowed the plasmid to be cleaved and the repair cassette to be linearized. One repair construct (Repair TFN T2A SEAP) included a T2A ribosome-skipping peptide sequence, and a sequence encoding a full-length transferrin signal peptide, before the SEAP transgene (FIG. 7A). A second repair construct (Repair TFN SEAP) without a T2A was also included (FIG. 7B). Constructs also included the TFN 19-20x.76 nuclease driven by a JeT promoter or a JeT promoter driving a truncated, nonfunctional TFN 19-20x.76 nuclease to serve as a control (truncTFN 19-20).

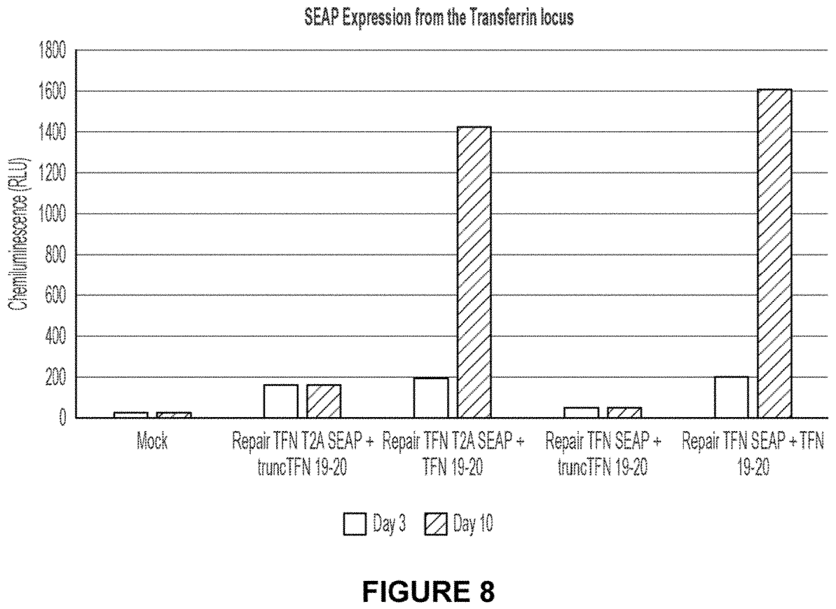

FL83B (murine liver cell line) cells were electroporated with repair constructs and assayed for SEAP expression in the conditioned media (FIG. 8). Both repair constructs that included the TFN 19-20x.76 nuclease yielded SEAP expression by ten days post electroporation. No significant difference was observed between the T2A peptide-containing construct (Repair TFN T2A SEAP; FIG. 7A) and the repair without a T2A (Repair TFN SEAP; FIG. 7B). As expected, neither of the repair constructs that contained truncated TFN 19-20x.76 nuclease (truncTFN 19-20) gave rise to significant SEAP expression.

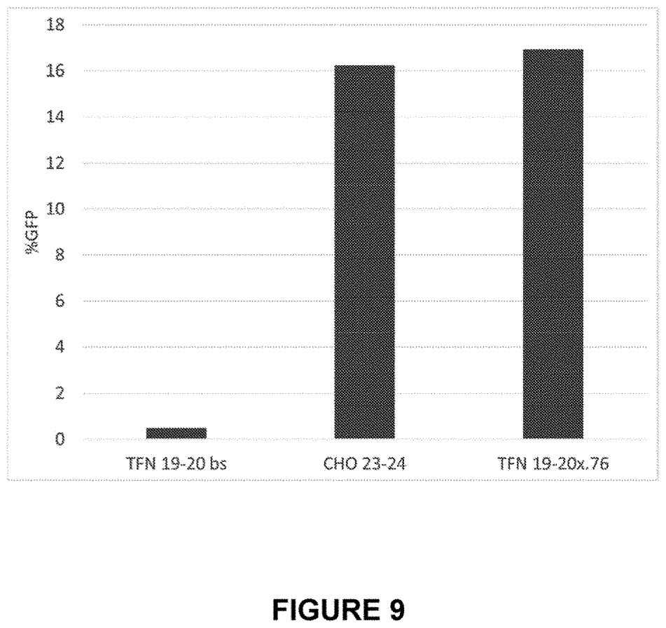

PCR was used to verify insertion of the SEAP transgene into the transferrin intron 1 (FIG. 9). The forward primer anneals to chromosomal sequence outside of the homology arm and the reverse primer anneals to the SEAP sequence. PCR yielded a distinct band that indicated integration of the repair cassette by HDR at 1113 bp for the Repair TFN SEAP construct (lane 3) or 1233 bp for the Repair TFN T2A SEAP plasmid (lane 5). These data suggest integration of the repair construct can be achieved by HDR and expression of the transgene is achieved from the transferrin promoter.

### C. Conclusions

Taken together, these data indicate the ability to insert a SEAP repair construct into the first intron of the transferrin gene. Using a splice acceptor on the 5′ end of the repair cassette allows for splicing from exon 1 to the transgene and subsequent secretion of the protein. When paired with a functional TFN 19-20x.76 nuclease, both repair constructs, with or without a T2A ribosomal skipping peptide, result in incorporation of the repair construct into the transferrin intron and SEAP expression in the conditioned media.

Interestingly, we observed a necessity for the remaining bases of the endogenous transferrin signal peptide that reside in exon 2. When these bases were excluded from repair constructs, and the signal peptide only comprised the amino acids encoded by exon 1, we observed cytotoxicity and little SEAP expression (data not shown). To our knowledge, this is the first time a transgene has been inserted and expressed from the first intron of the transferrin gene. The approach taken here has significant implications for therapeutic protein expression in vivo.

### Example 4

### In Vivo Experiment Demonstrating Use of Two AAVs to Insert a Construct into Transferrin Intron 1 and Express the Inserted Transgene in Mice

### A. Method

Molecular Cloning

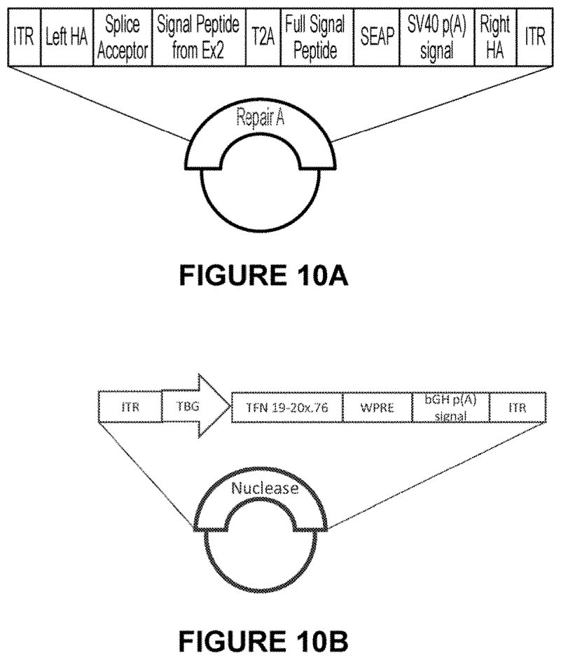

All AAV constructs were cloned using standard molecular biology cloning techniques. The repair AAV construct (referred to as “repair A AAV”; SEQ ID NO: 53; FIG. 10A) includes ˜500 bp homology arms flanking a repair cassette containing the splice acceptor sequence that precedes the endogenous transferrin exon 2, the remaining bases of the signal peptide in transferrin exon 2, a T2A ribosome-skipping peptide sequence, a sequence encoding a full-length transferrin signal peptide, and the SEAP transgene. A second AAV was cloned that carries the TFN 19-20x.76 nuclease driven by a liver-specific TBG promoter (referred to as “TFN 19-20 AAV”; SEQ ID NO: 57; FIG. 10B).

AAV Production

The adeno-associated virus (AAV) vector was generated using a tri-transfection protocol as previously described (Grieger et al., 2006, J. Nat Protocol). After iodixanol step gradient ultracentrifugation, AAV particles were purified through Q Sepharose chromatography, and the viral titer was determined by quantitative-PCR (qPCR).

Animal Husbandry and Serum Collection

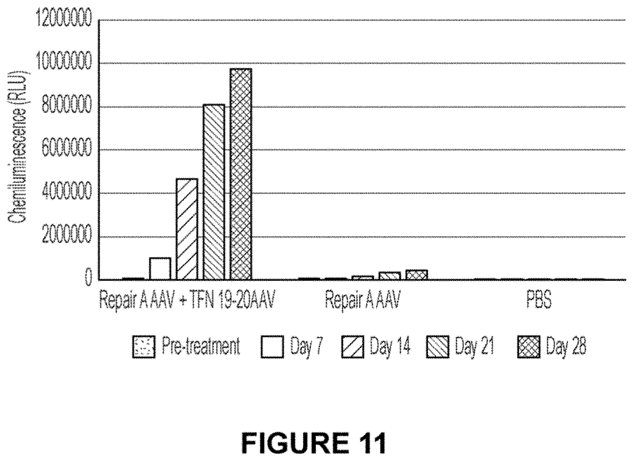

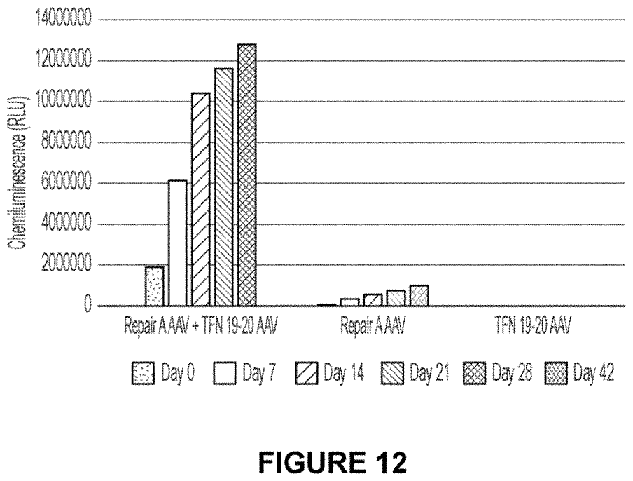

Seven-week-old FVB female mice were purchased from Jackson Laboratories (Bar Harbor, Me., USA). All animal work was performed with strict accordance with the recommendations in the Guide for the Care and Use of Laboratory Animals and all procedures were approved by the MisPro Biotech IACUC. Mice were placed in restraint with the tail positioned in a heated, lighted grove. The tail was swabbed with alcohol and using a 27-gauge insulin syringe, bevel side up, 200 μl of viral solution containing a mixture of PBS and rAAV was injected intravenously. Dosing for the cohort corresponding to data shown in FIG. 11 was 2.7×10¹² vg/mouse of the repair A AAV construct with or without 2.7×10¹¹ vg/mouse of the TFN19-20 AAV. Dosing for the cohort corresponding to data shown in FIG. 12 was 3×10¹² vg/mouse of the repair A AAV with or without 3×10¹¹ vg/mouse of the TFN19-20 AAV. PBS control mice received 200 μl PBS injections. After injection, mice were returned to their cages. Weekly blood collection was carried out by incising the submandibular vein with a sterile 4-mm lancet (MediPoint, Mineola, N.Y.), no more than 1% of animals body weight (approximately 100 ul) was collected into a serum separator tube (SST) (Becton, Dickinson and Company, Franklin Lakes, N.J.). Blood was centrifuged at 12,000 g for 5 minutes, and serum was stored at −20° C. until analysis.

SEAP Assay

Serum was assayed for SEAP expression using the Phospha light SEAP Reporter Gene Assay system (Thermo) according to the manufacturer's protocol. Serum was diluted 1:10 and chemiluminescence was measured using a Molecular Devices Spectramax i3 plate reader.

gDNA Extraction

gDNA was isolated from mouse livers 3 using the NucleoSpin Tissue kit from Machery-Nagel (ref #740952.250). The protocol was followed per kit manufacturer product manual. Briefly, a small section of liver was placed in a 1.5 ml tube. Lysis was achieved by incubation of the samples in a solution containing SDS and Proteinase K at 65° C. Appropriate conditions for binding of DNA to the silica membrane of the NucleoSpin® Tissue Columns were created by addition of large amounts of chaotropic ions and ethanol to the lysate. The binding process is reversible and specific to nucleic acids. Contaminations are removed by efficient washing with buffer. Pure genomic DNA is finally eluted under low ionic strength conditions in water.

Indel Assay (HDR ddPCR Assay)

Droplet digital PCR (ddPCR) assays were utilized to measure indels and target insertions (HDR) at the TFN19-20 binding site. The assay for indels used forward and reverse primers in the left and right homology arms and a FAM labeled probe complementary to the intact binding site. The assay to measure HDR was designed with a forward primer and FAM labelled probe in the mouse genome immediately outside of the left homology arm and a reverse primer in the SEAP gene. The reference for both assays used a primer pair that amplified a similar sized region ˜6 kb downstream of the binding site with a HEX probe for a genomic count control. Amplifications were multiplexed in a 20 μL reaction containing 1×ddPCR Supermix for Probes (no dUTP, BioRad), 250 nM of each probe, 900 nM of each primer, and ˜90 ng cellular gDNA. Droplets were generated using a QX100 droplet generator (BioRad). Amplification using previously optimized parameters was performed using a C1000 Touch thermal cycler (Bio-rad). Positive droplets were analysed using a Q200 droplet reader (Bio-Rad) and QuantaSoft Analysis Pro software (Bio-Rad). Primers used in the HDR and Indel ddPCR assays are provided in Table 5 below.

TABLE 5

| | |
|---|---|
| Binding site forward primer | CTGAGTCAGTAAGTCAGC |
| (SEQ ID NO: 38) | |
| Binding site reverse primer | GTCTTTCACTGAATCTGA |
| (SEQ ID NO: 39) | GC |
| Binding site probe (BHQ plus) | CCCAGTTGTACCATCTC |
| (SEQ ID NO: 40) | |
| HDR forward primer | TCATACCTGTATTTCTCC |
| (SEQ ID NO: 41) | TG |
| HDR reverse primer | GCTGGAGTTTCTTAGCAG |
| (SEQ ID NO: 42) | |
| HDR probe | CACAGGTCCCAGTCCTGC |
| (SEQ ID NO: 43) | ATCC |
| Indel Reference forward primer | CTAGACCTTTCAGTTCAT |
| (SEQ ID NO: 44) | GC |
| Indel Reference reverse primer | TGTCCAGATGTCAAAGAG |
| (SEQ ID NO: 45) | AT |
| Indel Reference probe | TTCAGCCTGCCACAGGCT |
| (SEQ ID NO: 46) | CAC |
| HDR Reference forward primer | TGTCTTTAACTGGTCATA |
| (SEQ ID NO: 47) | GG |
| HDR Reference reverse primer | GAACAAGAACTGAAGAGA |
| (SEQ ID NO: 48) | AC |
| HDR Reference probe | TTCAGCCTGCCACAGGCT |
| (SEQ ID NO: 49) | CAC |

### B. Results

To test whether transgene expression could be achieved using the endogenous transferrin promoter, a pair of AAV constructs was designed as described above (FIG. 10). FVB mice were dosed with the repair A AAV construct in combination with the TFN 19-20 AAV. Two different dosages of the repair A AAV and TFN 19-20 AAV were tested. The first lower dose level was 2.7×10¹² vg/mouse of the repair A AAV and 2.7×10¹¹ vg/mouse of the TFN 19-20 AAV, and the second higher dose level was 3×10¹² vg/mouse of the repair A AAV and 3×10¹¹ vg/mouse of the TFN 19-20 AAV. Additional control cohorts were included. For the first dose level, the control cohorts included a cohort of mice injected with PBS alone while the other cohort received repair A AAV alone. For the second dose level, the control cohorts included a cohort of mice injected with the TFN 19-20 AAV alone while the other cohort received repair A AAV alone. The repair alone cohort was not expected to produce SEAP unless there was expression from the repair cassette itself. The TFN 19-20 AAV was not expected to produce any expression because it did not contain a construct containing SEAP. Mouse serum was collected weekly, beginning the day prior to AAV dosing and continuing for four weeks post injections. Serum from each cohort at each timepoint was analyzed for SEAP expression. The SEAP expression results of the first and second dose levels are provided in FIG. 11 and FIG. 12, respectively. In both dose groups, control mice receiving the AAV repair alone showed very little SEAP protein, and in the first dose group, the PBS-injected mice showed no SEAP. As expected, in the second dose level mice receiving only the TFN 19-20 AAV showed no detectable SEAP expression. By contrast, mice that received both the repair A AAV and TFN 19-20 AAV produced significant SEAP in the serum as quickly as day 7 post-administration, with levels continuing to rise through day 28 of the study for dose level 1 and day 42 for dose level 2.

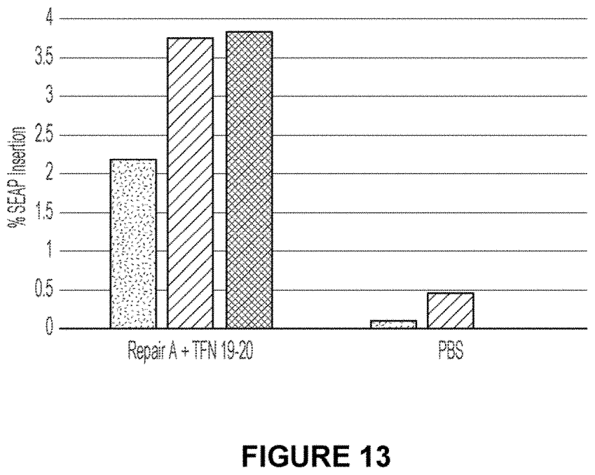

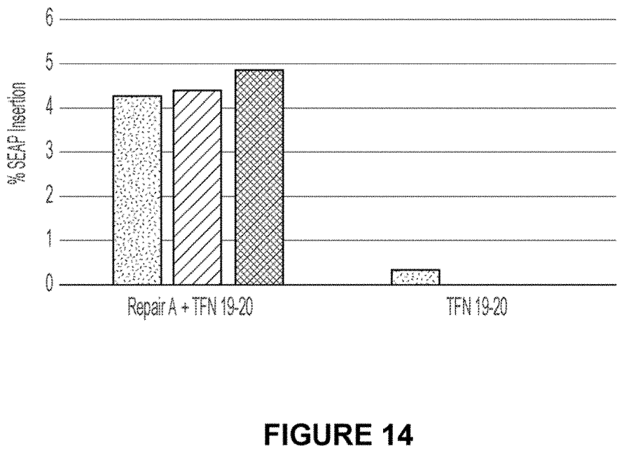

Analysis of the percentage of insertion of the repair A AAV construct at the TFN 19-20 binding site revealed between about 2% to about 4% insertion for the first dose level (FIG. 13) and between about 4% to about 5% insertion for the second dose level (FIG. 14) in three tested mice for each group. The PBS or nuclease only treated mice showed only baseline levels of the assay for SEAP insertion.

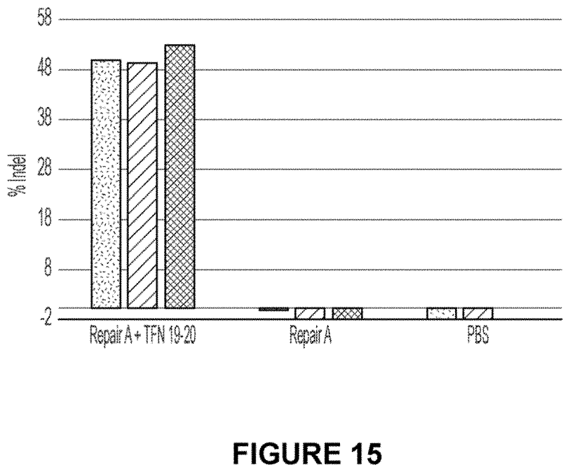

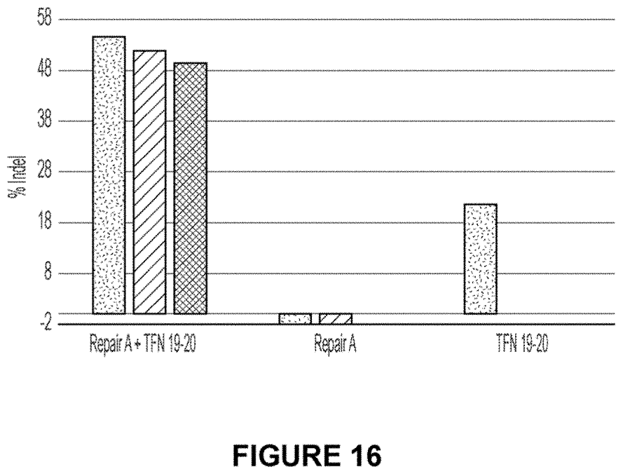

Analysis of the percentage indels at the TFN 19-20 binding site revealed between about 50% indels for the first dose level (FIG. 15) and between about 50% to about 55% indels for the second dose level (FIG. 16) in three tested mice for each group. By contrast, groups that received either the repair A AAV only or PBS showed no percent indel. One mice that received the nuclease alone at the second dose level demonstrated approximately 20% indels (FIG. 16).

### C. Conclusions

These data support the ability to express a donor transgene from the endogenous transferrin promoter at multiple doses. Additionally, in vitro data suggests it is possible to remove the T2A peptide sequence from the donor construct and use the endogenous transferrin signal peptide to drive SEAP secretion into the serum.

To our knowledge, this is the first demonstration in vivo of transgene expression driven from the endogenous transferrin promoter. Given the excess production of transferrin in humans, it is believed that utilizing the transferrin promoter to drive therapeutically-relevant protein production could be advantageous for patients with disease-causing protein aberrations (e.g., Pompe Disease).

### Example 5

### In Vivo Experiment Demonstrating Use of Additional Repair AAVs to Insert a Construct into Transferrin Intron 1 and Express the Inserted Transgene in Mice

### A. Method

Molecular Cloning

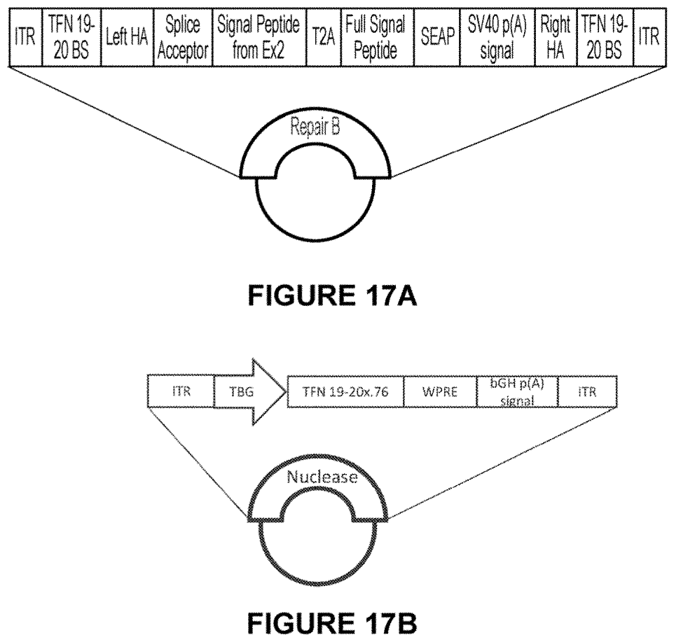

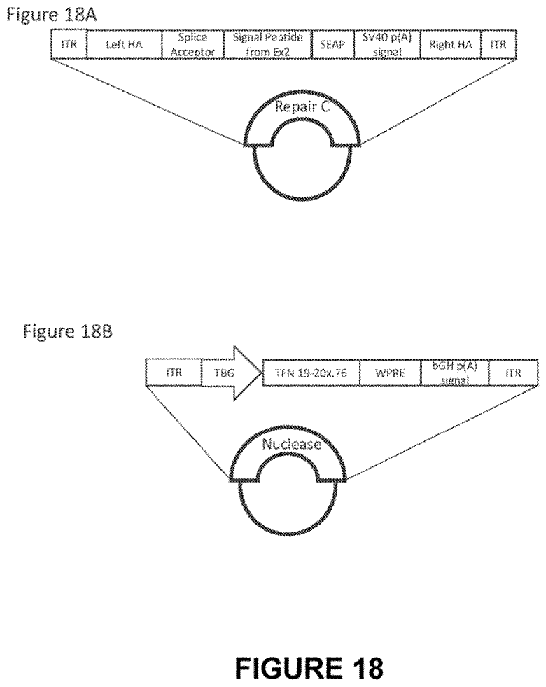

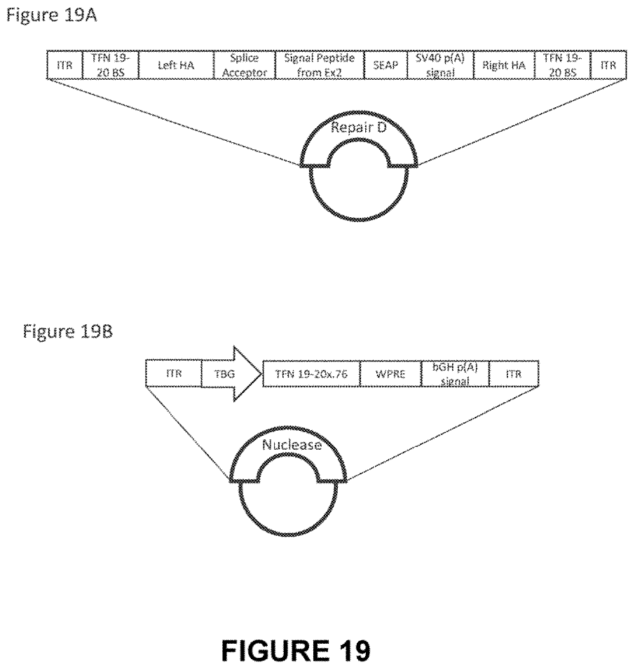

All AAV constructs were cloned using standard molecular biology cloning techniques. In this study, four different repair donor AAV constructs were tested. The first repair donor AAV construct is identical to the tested repair A AAV construct of Example 4 as shown in FIG. 10A. The second repair donor AAV construct includes inverted TFN 19-20 meganuclease binding sites flanking each of the 5′ and 3′ ˜500 bp homology arms, which flank a repair cassette containing the splice acceptor sequence that precedes the endogenous transferrin exon 2, the remaining bases of the signal peptide in transferrin exon 2, a T2A ribosome-skipping peptide sequence, a sequence encoding a full-length transferrin signal peptide, and the SEAP transgene (schematic shown as FIG. 17A and referred to as “repair B AAV”; SEQ ID NO: 54). The third repair donor AAV construct includes ˜500 bp homology arms flanking a repair cassette containing the splice acceptor sequence that precedes the endogenous transferrin exon 2, the remaining bases of the signal peptide in transferrin exon 2, and the SEAP transgene (schematic shown as FIG. 18A and referred to as “repair C AAV”; SEQ ID NO: 55). The fourth repair donor AAV construct includes inverted TFN 19-20 meganuclease binding sites flanking each of the 5′ and 3′ ˜500 bp homology arms, which flank a repair cassette containing the splice acceptor sequence that precedes the endogenous transferrin exon 2, the remaining bases of the signal peptide in transferrin exon 2, and the SEAP transgene (schematic shown as FIG. 19A and referred to as “repair D AAV”; SEQ ID NO: 56).

Animals were dosed with 3×10¹² vg/mouse of each respective repair AAV with or without 3×10¹¹ vg/mouse of the TFN19-20 AAV. PBS control mice received 200 μl PBS injections. The methods for AAV Production, Animal husbandry and serum collection, SEAP Assay, gDNA extraction, and Indel Assay (HDR ddPCR assay) were done as described in Example 4.

### B. Results

To test whether transgene expression could be achieved using the endogenous transferrin promoter, three additional repair AAV constructs (repair B AAV, repair C AAV, and repair D AAV) were designed in addition to the repair A AAV of Example 4 as described above (FIG. 10 and FIGS. 17-19).

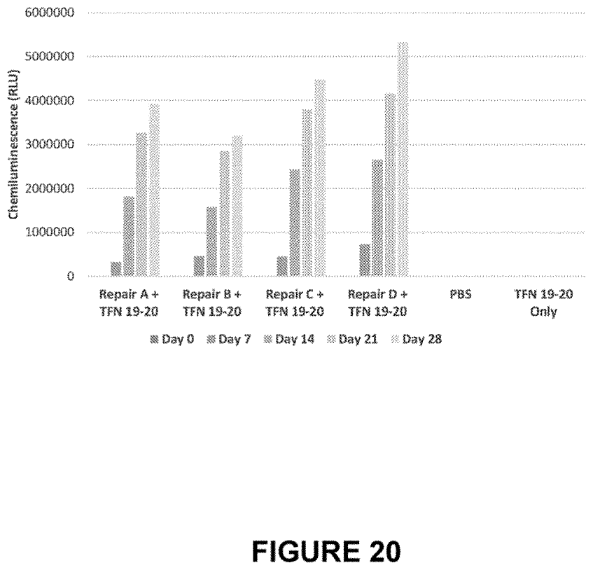

The results of SEAP expression for each repair and control cohort is provided in FIG. 20. Control mice receiving PBS or the TFN 19-20 AAV alone demonstrated no SEAP protein expression. By contrast, mice that received each of the four repair AAV constructs and the TFN 19-20 AAV produced significant SEAP in the serum as quickly as day 7 post-administration, with levels continuing to rise through day 28 with the repair D AAV demonstrating the highest levels of SEAP expression.

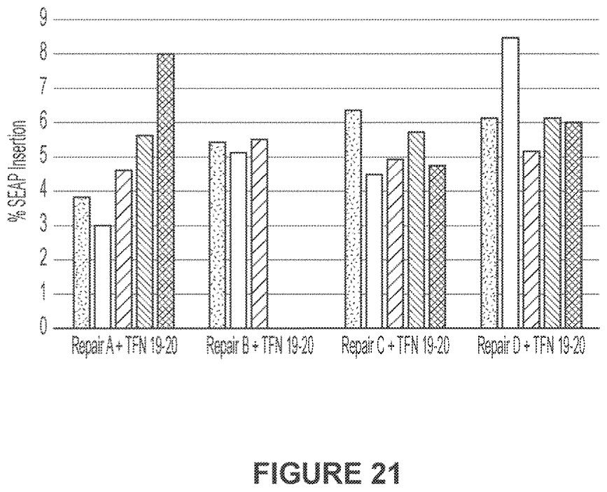

Analysis of the percentage of insertion of the repair A AAV construct at the TFN 19-20 binding site demonstrated about 3% to about 8% insertion. Repair B AAV consistently provided about 5% insertion of the repair construct. Repair C and repair D AAV provided between about 4% to about 6% or about 5% to about 8.5% insertion, respectively (FIG. 21).

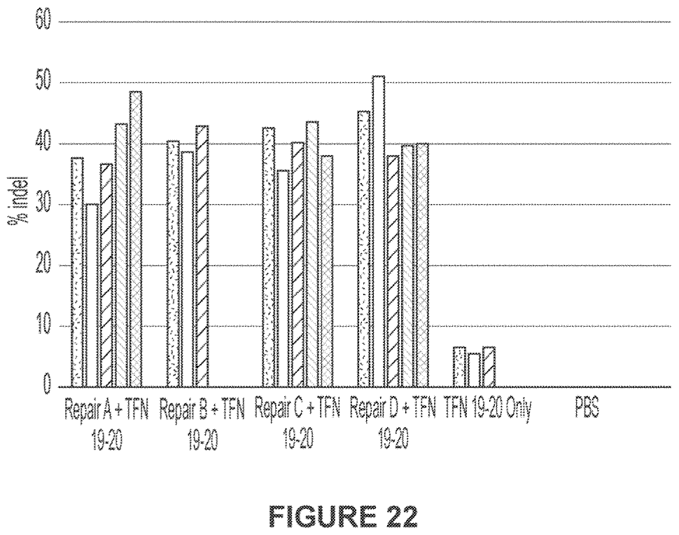

Analysis of the percent indels at the TFN 19-20 binding site revealed between about 30% to about 50% for the repair A AAV, about 40% for the repair B AAV, about 35% to about 45% for the repair C AAV, and about 40% to about 50% for the repair D AAV. Conversely, the PBS control showed no indels. Mice that received only the TFN 19-20 AAV control showed less than 10% indels (FIG. 22).

### C. Conclusions

These data further support the ability to express a donor transgene from the endogenous transferrin promoter and is consistent with the results provided in example 4. In addition, these data show that it is possible to remove the T2A peptide sequence from the repair construct and use the transferrin signal peptide from exon 2 as shown by the SEAP secretion into the serum (repair C and repair D AAV FIG. 20). Furthermore, it was demonstrated that the highest levels of SEAP protein secretion, insertion frequency, and percent indels was achieved with the repair D AAV, which includes two inverted TFN 19-20 binding sites and the transferrin signal peptide from exon 2.

1. An engineered meganuclease that binds and cleaves a recognition sequence within intron 1 of a transferrin gene, wherein said engineered meganuclease comprises a first subunit and a second subunit, wherein said first subunit binds to a first recognition half-site of said recognition sequence and comprises a first hypervariable (HVR1) region, and wherein said second subunit binds to a second recognition half-site of said recognition sequence and comprises a second hypervariable (HVR2) region.

2. The engineered meganuclease of claim 1, wherein said recognition sequence comprises SEQ ID NO: 19.

3. The engineered meganuclease of claim 1 or 2, wherein said HVR1 region comprises an amino acid sequence having at least 80% sequence identity to an amino acid sequence corresponding to residues 215-270 of SEQ ID NO: 23.

4. The engineered meganuclease of any one of claims 1-3, wherein said HVR1 region comprises one or more residues corresponding to residues 215, 217, 219, 221, 223, 224, 229, 231, 233, 235, 237, 259, 261, 266, and 268 of SEQ ID NO: 23.

5. The engineered meganuclease of any one of claims 1-4, wherein said HVR1 region comprises Y, R, K, or D at a residue corresponding to residue 257 of SEQ ID NO: 23.

6. The engineered meganuclease of any one of claims 1-5, wherein said HVR1 region comprises residues 215-270 of SEQ ID NO: 23.

7. The engineered meganuclease of any one of claims 1-6, wherein said first subunit comprises an amino acid sequence having at least 80% sequence identity to residues 198-344 of SEQ ID NO: 23.

8. The engineered meganuclease of any one of claims 1-7, wherein said first subunit comprises G, S, or A at a residue corresponding to residue 210 of any one of SEQ ID NO: 23.

9. The engineered meganuclease of any one of claims 1-8, wherein said first subunit comprises E, Q, or K at a residue corresponding to residue 271 of SEQ ID NO: 23.

10. The engineered meganuclease of any one of claims 1-9, wherein said first subunit comprises a residue corresponding to residue 271 of SEQ ID NO: 23.

11. The engineered meganuclease of any one of claims 1-10, wherein said first subunit comprises residues 198-344 of SEQ ID NO: 23.

12. The engineered meganuclease of any one of claims 1-11, wherein said HVR2 region comprises an amino acid sequence having at least 80% sequence identity to an amino acid sequence corresponding to residues 24-79 of SEQ ID NO: 23.

13. The engineered meganuclease of any one of claims 1-12, wherein said HVR2 region comprises one or more residues corresponding to residues 24, 26, 28, 30, 32, 33, 38, 40, 42, 44, 46, 68, 70, 75, and 77 of SEQ ID NO: 23.

14. The engineered meganuclease of any one of claims 1-13, wherein said HVR2 region comprises a residue corresponding to residue 41 of SEQ ID NO: 23.

15. The engineered meganuclease of any one of claims 1-14, wherein said HVR2 region comprises Y, R, K, or D at a residue corresponding to residue 66 of SEQ ID NO: 23.

16. The engineered meganuclease of any one of claims 1-15, wherein said HVR2 region comprises residues 24-79 of SEQ ID NO: 23.

17. The engineered meganuclease of any one of claims 1-16, wherein said second subunit comprises an amino acid sequence having at least 80% sequence identity to residues 7-153 of SEQ ID NO: 23.

18. The engineered meganuclease of any one of claims 1-17, wherein said second subunit comprises G, S, or A at a residue corresponding to residue 19 of SEQ ID NO: 23.

19. The engineered meganuclease of any one of claims 1-18, wherein said second subunit comprises E, Q, or K at a residue corresponding to residue 80 of SEQ ID NO: 23.

20. The engineered meganuclease of any one of claims 1-19, wherein said second subunit comprises a residue corresponding to residue 80 of SEQ ID NO: 23.

21. The engineered meganuclease of any one of claims 1-20, wherein said second subunit comprises residues 7-153 of any one of SEQ ID NO: 23.

22. The engineered meganuclease of any one of claims 1-21, wherein said engineered meganuclease comprises a linker, wherein said linker covalently joins said first subunit and said second subunit.

23. The engineered meganuclease of any one of claims 1-22, wherein said engineered meganuclease comprises the amino acid sequence of SEQ ID NO: 23.

24. The engineered meganuclease of claim 1, wherein said recognition sequence comprises SEQ ID NO: 21.

25. The engineered meganuclease of claim 24, wherein said HVR1 region comprises an amino acid sequence having at least 80% sequence identity to an amino acid sequence corresponding to residues 215-270 of SEQ ID NO: 26.

26. The engineered meganuclease of claim 24 or claim 25, wherein said HVR1 region comprises one or more residues corresponding to residues 215, 217, 219, 221, 223, 224, 229, 231, 233, 235, 237, 259, 261, 266, and 268 of SEQ ID NO: 26.

27. The engineered meganuclease of any one of claims 24-26, wherein said HVR1 region comprises Y, R, K, or D at a residue corresponding to residue 257 of SEQ ID NO: 26.

28. The engineered meganuclease of any one of claims 24-27, wherein said HVR1 region comprises residues 215-270 of SEQ ID NO: 26.

29. The engineered meganuclease of any one of claims 24-28, wherein said first subunit comprises an amino acid sequence having at least 80% sequence identity to residues 198-344 of SEQ ID NO: 26.

30. The engineered meganuclease of any one of claims 24-29, wherein said first subunit comprises G, S, or A at a residue corresponding to residue 210 of SEQ ID NO: 26.

31. The engineered meganuclease of any one of claims 24-30, wherein said first subunit comprises E, Q, or K at a residue corresponding to residue 271 of SEQ ID NO: 26.

32. The engineered meganuclease of any one of claims 24-31, wherein said first subunit comprises a residue corresponding to residue 271 of SEQ ID NO: 26.

33. The engineered meganuclease of any one of claims 24-32, wherein said first subunit comprises residues 198-344 of SEQ ID NO: 26.

34. The engineered meganuclease of any one of claims 24-33, wherein said HVR2 region comprises an amino acid sequence having at least 80% sequence identity to an amino acid sequence corresponding to residues 24-79 of SEQ ID NO: 26.

35. The engineered meganuclease of any one of claims 24-34, wherein said HVR2 region comprises one or more residues corresponding to residues 24, 26, 28, 30, 32, 33, 38, 40, 42, 44, 46, 68, 70, 75, and 77 of SEQ ID NO: 26.

36. The engineered meganuclease of any one of claims 24-35, wherein said HVR2 region comprises Y, R, K, or D at a residue corresponding to residue 66 of SEQ ID NO: 26.

37. The engineered meganuclease of any one of claims 24-36, wherein said HVR2 region comprises residues 24-79 of SEQ ID NO: 26.

38. The engineered meganuclease of any one of claims 24-37, wherein said second subunit comprises an amino acid sequence having at least 80% sequence identity to residues 7-153 of SEQ ID NO: 26.

39. The engineered meganuclease of any one of claims 24-38, wherein said second subunit comprises G, S, or A at a residue corresponding to residue 210 of SEQ IDs NO: 26.

40. The engineered meganuclease of any one of claims 24-39, wherein said second subunit comprises E, Q, or K at a residue corresponding to residue 271 of SEQ ID NOs: 26.

41. The engineered meganuclease of any one of claims 24-40, wherein said second subunit comprises a residue corresponding to residue 80 of SEQ ID NO: 26.

42. The engineered meganuclease of any one of claims 24-41, wherein said second subunit comprises residues 7-153 of any one of SEQ ID NOs: 26.

43. The engineered meganuclease of any one of claims 24-42, wherein said engineered meganuclease comprises a linker, wherein said linker covalently joins said first subunit and said second subunit.

44. The engineered meganuclease of any one of claims 24-43, wherein said engineered meganuclease comprises the amino acid sequence of SEQ ID NO: 26.

45. A polynucleotide comprising a nucleic acid sequence encoding said engineered meganuclease of any one of claims 1-44.

46. The polynucleotide of claim 45, wherein said polynucleotide is an mRNA.

47. A recombinant DNA construct comprising a nucleic acid sequence encoding said engineered meganuclease of any one of claims 1-44.

48. The recombinant DNA construct of claim 47, wherein said recombinant DNA construct encodes a viral vector comprising said nucleic acid sequence encoding said engineered meganuclease.

49. The recombinant DNA construct of claim 48, wherein said viral vector is an adenoviral vector, a lentiviral vector, a retroviral vector, or an adeno-associated viral (AAV) vector.

50. The recombinant DNA construct of claim 48 or 49, wherein said viral vector is a recombinant AAV vector.

51. A viral vector comprising a nucleic acid sequence encoding said engineered meganuclease of any one of claims 1-44.

52. The viral vector of claim 51, wherein said viral vector is an adenoviral vector, a lentiviral vector, a retroviral vector, or an adeno-associated viral (AAV) vector.

53. The viral vector of claim 52, wherein said viral vector is a recombinant AAV vector.

54. A method for producing a genetically-modified eukaryotic cell comprising an exogenous nucleic acid molecule encoding a polypeptide of interest inserted into a chromosome of said eukaryotic cell, said method comprising introducing into a eukaryotic cell one or more nucleic acids including:
(a) a nucleic acid encoding said engineered meganuclease of any one of claims 1-44, wherein said engineered meganuclease is expressed in said eukaryotic cell; and
(b) a template nucleic acid comprising said exogenous nucleic acid molecule;
wherein said engineered meganuclease produces a cleavage site in said chromosome at a recognition sequence comprising SEQ ID NO: 19 or 21;
and wherein said exogenous nucleic acid molecule is inserted into said chromosome at said cleavage site.

55. The method of claim 54, wherein said exogenous nucleic acid molecule further comprises sequences homologous to sequences flanking said cleavage site and said exogenous nucleic acid molecule is inserted at said cleavage site by homologous recombination.

56. The method of claim 54 or claim 55, wherein said eukaryotic cell is a mammalian cell.

57. The method of claim 56, wherein said mammalian cell is selected from a human cell, non-human primate cell, or a mouse cell.

58. The method of claim 56 or claim 57, wherein said mammalian cell is a hepatocyte.

59. The method of claim 58, wherein said hepatocyte is within the liver of a human, a non-human primate, or a mouse.

60. The method of any one of claims 54-59, wherein said nucleic acid encoding said engineered meganuclease is introduced into said eukaryotic cell by an mRNA or a viral vector.

61. The method of any one of claims 54-60, wherein said template nucleic acid is introduced into said eukaryotic cell by a viral vector.

62. A method for producing a genetically-modified eukaryotic cell comprising an exogenous nucleic acid molecule encoding a polypeptide of interest inserted into a chromosome of said eukaryotic cell, said method comprising:
(a) introducing said engineered meganuclease of any one of claims 1-44 into a eukaryotic cell; and
(b) introducing a template nucleic acid comprising said exogenous nucleic acid molecule into said eukaryotic cell;
wherein said engineered meganuclease produces a cleavage site in said chromosome at a recognition sequence comprising SEQ ID NO: 19 or 21;
and wherein said exogenous nucleic acid molecule is inserted into said chromosome at said cleavage site.

63. The method of claim 62, wherein said exogenous nucleic acid molecule further comprises sequences homologous to sequences flanking said cleavage site and said exogenous nucleic acid molecule is inserted at said cleavage site by homologous recombination.

64. The method of claim 62 or claim 63, wherein said eukaryotic cell is a mammalian cell.

65. The method of claim 64, wherein said mammalian cell is selected from a human cell, non-human primate cell, or a mouse cell.

66. The method of claim 64 or claim 65, wherein said mammalian cell is a hepatocyte.

67. The method of claim 66, wherein said hepatocyte is within the liver of a human, a non-human primate, or a mouse.

68. The method of any one of claims 62-67, wherein said template nucleic acid is introduced into said eukaryotic cell by a viral vector.

69. A genetically-modified eukaryotic cell prepared by the method of any one of claims 54-68.

70. A nucleic acid molecule comprising, from 5′ to 3′:
(a) an exogenous splice acceptor sequence;
(b) a first nucleic acid sequence encoding a C-terminal fragment of a signal peptide;
(c) a second nucleic acid sequence encoding an exogenous polypeptide of interest; and
(d) a polyA signal.

71. The nucleic acid molecule of claim 70, wherein said first nucleic acid sequence is capable of being joined directly to the 3′ end of SEQ ID NO: 8 to generate a coding sequence for a transferrin signal peptide having at least 80% sequence identity to SEQ ID NO: 7.

72. The nucleic acid molecule of claim 70 or claim 71, wherein said first nucleic acid sequence has at least 80% sequence identity to SEQ ID NO: 9.

73. The nucleic acid molecule of any one of claims 70-72, wherein said first nucleic acid sequence comprises SEQ ID NO: 9.

74. The nucleic acid molecule of any one of claims 70-73, further comprising a 5′ homology arm which is positioned 5′ upstream of said exogenous splice acceptor sequence, and a 3′ homology arm which is positioned 3′ downstream of said polyA signal, wherein said 5′ homology arm and said 3′ homology arm are homologous to sequences flanking an engineered nuclease cleavage site of interest within intron 1 of a transferrin gene.

75. The nucleic acid molecule of any one of claims 70-74, wherein said exogenous splice acceptor sequence has at least 80% sequence identity to SEQ ID NO: 10.

76. The nucleic acid molecule of any one of claims 70-75, wherein said exogenous splice acceptor sequence comprises SEQ ID NO: 10.

77. The nucleic acid molecule of any one of claims 71-74, wherein said exogenous splice acceptor sequence is not derived from intron 1 of said transferrin gene.

78. The nucleic acid molecule of any one of claims 70-77, wherein said nucleic acid molecule comprises, from 5′ to 3′:
(a) said exogenous splice acceptor sequence;
(b) said first nucleic acid sequence;
(c) a 2A sequence or IRES sequence;
(d) a third nucleic acid sequence encoding a signal peptide;
(e) said second nucleic acid sequence; and
(f) said polyA signal.

79. The nucleic acid molecule of claim 78, wherein said signal peptide encoded by said third nucleic acid sequence comprises an amino acid sequence having at least 80% sequence identity to SEQ ID NO: 7.

80. The nucleic acid molecule of claim 78 or claim 79, wherein said signal peptide encoded by said third nucleic acid sequence comprises an amino acid sequence of SEQ ID NO: 7.

81. The nucleic acid molecule of any one of claims 70-80, wherein said exogenous polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

82. The nucleic acid molecule of any one of claims 70-81, wherein said polyA signal comprises a nucleic acid sequence having at least 80% sequence identity to SEQ ID NO: 34.

83. The nucleic acid molecule of any one of claims 70-82, wherein said polyA signal comprises a nucleic acid sequence of SEQ ID NO: 34.

84. A genetically-modified eukaryotic cell comprising a modified transferrin gene, wherein said modified transferrin gene comprises an exogenous nucleic acid molecule within intron 1, and wherein said exogenous nucleic acid molecule comprises, from 5′ to 3′:
(a) an exogenous splice acceptor sequence;
(b) a first nucleic acid sequence encoding a C-terminal fragment of a signal peptide; and
(c) a second nucleic acid sequence encoding a polypeptide of interest; and
(d) a polyA signal.

85. The genetically-modified eukaryotic cell of claim 84, wherein said first nucleic acid sequence is capable of being joined directly to the 3′ end of SEQ ID NO: 8 to generate a coding sequence for a transferrin signal peptide having at least 80% sequence identity to SEQ ID NO: 7.

86. The genetically-modified eukaryotic cell of claim 84 or claim 85, wherein said first nucleic acid sequence has at least 80% sequence identity to SEQ ID NO: 9.

87. The genetically-modified eukaryotic cell of any one of claims 84-86, wherein said first nucleic acid sequence comprises SEQ ID NO: 9.

88. The genetically-modified eukaryotic cell of any one of claims 84-87, wherein said exogenous splice acceptor sequence has at least 80% sequence identity to SEQ ID NO: 10.

89. The genetically-modified eukaryotic cell of any one of claims 84-88, wherein said exogenous splice acceptor sequence comprises SEQ ID NO: 10.

90. The genetically-modified eukaryotic cell of any one of claims 84-87, wherein said exogenous splice acceptor sequence is not derived from intron 1 of said transferrin gene.

91. The genetically-modified eukaryotic cell of any one of claims 84-90, wherein said exogenous nucleic acid molecule comprises, from 5′ to 3′:
(a) said exogenous splice acceptor sequence;
(b) said first nucleic acid sequence;
(c) a 2A sequence or IRES sequence;
(d) a third nucleic acid sequence encoding a signal peptide;
(e) said second nucleic acid sequence; and
(f) said polyA signal.

92. The genetically-modified eukaryotic cell of claim 91, wherein said signal peptide encoded by said third nucleic acid sequence comprises an amino acid sequence having at least 80% sequence identity to SEQ ID NO: 7.

93. The genetically-modified eukaryotic cell of claim 91 or claim 92, wherein said signal peptide encoded by said third nucleic acid sequence comprises an amino acid sequence of SEQ ID NO: 7.

94. The genetically-modified eukaryotic cell of any one of claims 84-93, wherein said polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

95. The genetically-modified eukaryotic cell of any one of claims 84-94, wherein said polyA signal comprises a nucleic acid sequence having at least 80% sequence identity to SEQ ID NO: 34.

96. The genetically-modified eukaryotic cell of any one of claims 84-95, wherein said polyA signal comprises a nucleic acid sequence of SEQ ID NO: 34.

97. The genetically-modified eukaryotic cell of any one of claims 84-96, wherein an endogenous promoter of said modified transferrin gene is operably linked to said exogenous nucleic acid molecule.

98. The genetically-modified eukaryotic cell of any one of claims 84-97, wherein said endogenous promoter of said transferrin gene drives expression of said exogenous nucleic acid molecule.

99. The genetically-modified eukaryotic cell of any one of claims 84-98, wherein said genetically-modified eukaryotic cell expresses a polypeptide comprising:
(a) a transferrin signal peptide having at least 80% sequence identity to SEQ ID NO: 7; and
(b) said polypeptide of interest;
wherein said polypeptide of interest is secreted by said genetically-modified eukaryotic cell.

100. The genetically-modified eukaryotic cell of any one of claims 84-99, wherein said exogenous nucleic acid molecule is positioned within intron 1 at an engineered nuclease cleavage site.

101. The genetically-modified eukaryotic cell of claim 100, wherein said engineered nuclease cleavage site is within an engineered meganuclease recognition sequence, a TALEN recognition sequence, a compact TALEN recognition sequence, a megaTAL recognition sequence, a zinc finger nuclease recognition sequence, or a CRISPR system nuclease recognition sequence.

102. The genetically-modified eukaryotic cell of claim 100 or claim 101, wherein said engineered nuclease cleavage site is within an engineered meganuclease recognition sequence.

103. The genetically-modified eukaryotic cell of claim 102, wherein said engineered meganuclease recognition sequence comprises SEQ ID NO: 19 or 21.

104. The genetically-modified eukaryotic cell of claim 100 or 101, wherein said engineered nuclease cleavage site is a TALEN cleavage site within a TALEN spacer sequence.

105. The genetically-modified eukaryotic cell of claim 100 or 101, wherein said engineered nuclease cleavage site is a zinc finger nuclease cleavage site within a zinc finger nuclease spacer sequence.

106. The genetically-modified eukaryotic cell of claim 100 or 101, wherein said engineered nuclease cleavage site is within a CRISPR system nuclease recognition sequence.

107. The genetically-modified eukaryotic cell of any one of claims 84-106, wherein said eukaryotic cell is a mammalian cell.

108. The genetically-modified eukaryotic cell of claim 107, wherein said mammalian cell is selected from a human cell, non-human primate cell, or a mouse cell.

109. The genetically-modified eukaryotic cell of claim 107 or claim 108, wherein said mammalian cell is a hepatocyte.

110. The genetically-modified eukaryotic cell of claim 109, wherein said hepatocyte is within the liver of a human, a non-human primate, or a mouse.

111. A pharmaceutical composition comprising a pharmaceutically-acceptable carrier and a therapeutically effective amount of:
(a) a nucleic acid encoding an engineered nuclease having specificity for a recognition sequence within intron 1 of a transferrin gene; and
(b) a template nucleic acid comprising an exogenous nucleic acid molecule, wherein said exogenous nucleic acid molecule comprises, from 5′ to 3′:
(i) an exogenous splice acceptor sequence;
(ii) a first nucleic acid sequence encoding a C-terminal fragment of a signal peptide;
(iii) a second nucleic acid sequence encoding a polypeptide of interest; and
(iv) a polyA signal.

112. The pharmaceutical composition of claim 111, wherein said first nucleic acid sequence is capable of being joined directly to the 3′ end of SEQ ID NO: 8 to generate a coding sequence for a transferrin signal peptide having at least 80% sequence identity to SEQ ID NO: 7.

113. The pharmaceutical composition of claim 111 or claim 112, wherein said first nucleic acid sequence has at least 80% sequence identity to SEQ ID NO: 9.

114. The pharmaceutical composition of any one of claims 111-113, wherein said first nucleic acid sequence comprises SEQ ID NO: 9.

115. The pharmaceutical composition of any one of claims 111-114, wherein said exogenous nucleic acid molecule further comprises a 5′ homology arm which is positioned 5′ upstream of said exogenous splice acceptor sequence, and a 3′ homology arm which is positioned 3′ downstream of said polyA signal, wherein said 5′ homology arm and said 3′ homology arm are homologous to sequences flanking a cleavage site generated by said engineered nuclease within intron 1 of said transferrin gene.

116. The pharmaceutical composition of any one of claims 111-115, wherein said exogenous splice acceptor sequence has at least 80% sequence identity to SEQ ID NO: 10.

117. The pharmaceutical composition of claim 116, wherein said exogenous splice acceptor sequence comprises SEQ ID NO: 10.

118. The pharmaceutical composition of any one of claims 111-116, wherein said exogenous splice acceptor sequence is not derived from intron 1 of said transferrin gene.

119. The pharmaceutical composition of any one of claims 111-118, wherein said exogenous nucleic acid molecule comprises, from 5′ to 3′:
(a) said exogenous splice acceptor sequence;
(b) said first nucleic acid sequence;
(c) a 2A sequence or IRES sequence;
(d) a third nucleic acid sequence encoding a signal peptide;
(e) said second nucleic acid sequence; and
(f) said polyA signal.

120. The pharmaceutical composition of claim 119, wherein said signal peptide encoded by said third nucleic acid sequence comprises an amino acid sequence having at least 80% sequence identity to SEQ ID NO: 7.

121. The pharmaceutical composition of claim 120, wherein said signal peptide encoded by said third nucleic acid sequence comprises an amino acid sequence of SEQ ID NO: 7.

122. The pharmaceutical composition of any one of claims 111-121, wherein said polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

123. The pharmaceutical composition of any one of claims 111-122, wherein said polyA signal comprises a nucleic acid sequence having at least 80% sequence identity to SEQ ID NO: 34.

124. The pharmaceutical composition of any one of claims 111-123, wherein said polyA signal comprises a nucleic acid sequence of SEQ ID NO: 34.

125. The pharmaceutical composition of any one of claims 111-124, wherein said nucleic acid encoding said engineered nuclease is an mRNA.

126. The pharmaceutical composition of claim 125, wherein said mRNA is packaged within a lipid nanoparticle.

127. The pharmaceutical composition of any one of claims 111-126, wherein a viral vector comprises said template nucleic acid.

128. The pharmaceutical composition of claim 127, wherein a recombinant AAV vector comprises said template nucleic acid.

129. The pharmaceutical composition of any one of claims 111-128, wherein said pharmaceutical composition comprises a therapeutically effective amount of:
(a) an mRNA encoding said engineered nuclease, wherein said mRNA is packaged within a lipid nanoparticle; and
(b) a recombinant AAV vector comprising said template nucleic acid.

130. The pharmaceutical composition of claim 129, wherein said pharmaceutical composition comprises:
(a) only one population of lipid nanoparticles comprising said mRNA encoding said engineered nuclease; and
(b) only one population of recombinant AAV vectors comprising said template nucleic acid.

131. The pharmaceutical composition of any one of claims 111-124, wherein a first viral vector comprises said nucleic acid encoding said engineered nuclease.

132. The pharmaceutical composition of claim 131, wherein a first recombinant AAV vector comprises said nucleic acid encoding said engineered nuclease.

133. The pharmaceutical composition of claim 131 or 132, wherein a second viral vector comprises said template nucleic acid.

134. The pharmaceutical composition of claim 133, wherein a second recombinant AAV vector comprises said template nucleic acid.

135. The pharmaceutical composition of any one of claims 111-124 and 131-134, wherein said pharmaceutical composition comprises only two populations of viral vectors, wherein a first population of viral vectors comprises said nucleic acid encoding said engineered nuclease, and wherein a second population of viral vectors comprises said template nucleic acid.

136. The pharmaceutical composition of claim 135, wherein said pharmaceutical composition comprises:
(a) a first population of recombinant AAV vectors comprising said nucleic acid encoding said engineered nuclease; and
(b) a second population of recombinant AAV vectors comprising said template nucleic acid.

137. The pharmaceutical composition of any one of claims 111-136, wherein said engineered nuclease is an engineered meganuclease, a TALEN, a compact TALEN, a megaTAL, a zinc finger nuclease, or a CRISPR system nuclease.

138. The pharmaceutical composition of claim 137, wherein said engineered nuclease is an engineered meganuclease having specificity for a meganuclease recognition sequence within intron 1 of said transferrin gene.

139. The pharmaceutical composition of claim 138, wherein said meganuclease recognition sequence comprises SEQ ID NO: 19 or 21.

140. The pharmaceutical composition of claim 139, wherein said engineered nuclease is said engineered meganuclease of any one of claims 1-44.

141. The pharmaceutical composition of claim 137, wherein said engineered nuclease is a TALEN having specificity for a TALEN recognition sequence within intron 1 of said transferrin gene.

142. The pharmaceutical composition of claim 137, wherein said engineered nuclease is a zinc finger nuclease having specificity for a zinc finger nuclease recognition sequence within intron 1 of said transferrin gene.

143. The pharmaceutical composition of claim 137, wherein said engineered nuclease is a CRISPR system nuclease having specificity for a recognition sequence within intron 1 of said transferrin gene.

144. The pharmaceutical composition of any one of claims 111-143, wherein said polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

145. A method for producing a genetically-modified eukaryotic cell comprising a modified transferrin gene, said method comprising introducing into a eukaryotic cell:
(a) a nucleic acid encoding an engineered nuclease having specificity for a recognition sequence within intron 1 of a transferrin gene, wherein said engineered nuclease is expressed in said eukaryotic cell; and
(b) a template nucleic acid comprising an exogenous nucleic acid molecule, wherein said exogenous nucleic acid molecule comprises, from 5′ to 3′:
(i) an exogenous splice acceptor sequence;
(ii) a first nucleic acid sequence encoding a C-terminal fragment of a signal peptide;
(iii) a second nucleic acid sequence encoding a polypeptide of interest; and
(iv) a polyA signal;
wherein said engineered nuclease produces a cleavage site at said recognition sequence, and wherein said exogenous nucleic acid molecule is inserted into intron 1 of said transferrin gene at said cleavage site, thereby generating said modified transferrin gene in said eukaryotic cell.

146. The method of claim 145, wherein said first nucleic acid sequence is capable of being joined directly to the 3′ end of SEQ ID NO: 8 to generate a coding sequence for a transferrin signal peptide having at least 80% sequence identity to SEQ ID NO: 7.

147. The method of claim 145 or claim 146, wherein said first nucleic acid sequence has at least 80% sequence identity to SEQ ID NO: 9.

148. The method of any one of claims 145-147, wherein said first nucleic acid sequence comprises SEQ ID NO: 9.

149. The method of any one of claims 145-148, wherein said exogenous nucleic acid molecule further comprises a 5′ homology arm which is positioned 5′ upstream of said exogenous splice acceptor sequence, and a 3′ homology arm which is positioned 3′ downstream of said polyA signal, wherein said 5′ homology arm and said 3′ homology arm are homologous to sequences flanking said cleavage site, and wherein said exogenous nucleic acid molecule is inserted into said cleavage site by homologous recombination.

150. The method of any one of claims 145-149, wherein upon generation of said modified transferrin gene, the endogenous promoter of said transferrin gene is operably linked to said exogenous nucleic acid molecule.

151. The method of any one of claims 145-150, wherein said endogenous promoter of said transferrin gene drives expression of said exogenous nucleic acid molecule.

152. The method of any one of claims 145-151, wherein said genetically-modified eukaryotic cell expresses a polypeptide comprising:
(a) a transferrin signal peptide having at least 80% sequence identity to SEQ ID NO: 7; and
(b) said polypeptide of interest;
wherein said polypeptide of interest is secreted by said genetically-modified eukaryotic cell.

153. The method of any one of claims 145-152, wherein said exogenous splice acceptor sequence has at least 80% sequence identity to SEQ ID NO: 10.

154. The method of any one of claims 145-153, wherein said exogenous splice acceptor sequence comprises SEQ ID NO: 10.

155. The method of any one of claims 145-152, wherein said exogenous splice acceptor sequence is not derived from intron 1 of said transferrin gene.

156. The method of any one of claims 145-155, wherein said exogenous nucleic acid molecule comprises, from 5′ to 3′:
(a) said exogenous splice acceptor sequence;
(b) said first nucleic acid sequence;
(c) a 2A sequence or IRES sequence;
(d) a third nucleic acid sequence encoding a signal peptide;
(e) said second nucleic acid sequence; and
(f) said polyA signal.

157. The method of claim 156, wherein said signal peptide encoded by said third nucleic acid sequence comprises an amino acid sequence having at least 80% sequence identity to SEQ ID NO: 7.

158. The method of claim 157, wherein said signal peptide encoded by said third nucleic acid sequence comprises an amino acid sequence of SEQ ID NO: 7.

159. The method of any one of claims 145-158, wherein said polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

160. The method of any one of claims 145-159, wherein said polyA signal comprises a nucleic acid sequence having at least 80% sequence identity to SEQ ID NO: 34.

161. The method of any one of claims 145-160, wherein said polyA signal comprises a nucleic acid sequence of SEQ ID NO: 34.

162. The method of any one of claims 145-161, wherein said nucleic acid encoding said engineered nuclease is an mRNA.

163. The method of claim 162, wherein said mRNA is packaged within a lipid nanoparticle.

164. The method of any one of claims 145-163, wherein a viral vector comprises said template nucleic acid.

165. The method of claim 164, wherein a recombinant AAV vector comprises said template nucleic acid.

166. The method of any one of claims 145-165, said method comprising contacting said eukaryotic cell with:
(a) a lipid nanoparticle comprising an mRNA encoding said engineered nuclease; and
(b) a recombinant AAV vector comprising said template nucleic acid.

167. The method of claim 166, wherein said eukaryotic cell is contacted with:
(a) only one population of lipid nanoparticles comprising said mRNA encoding said engineered nuclease; and
(b) only one population of recombinant AAV vectors comprising said template nucleic acid.

168. The method of any one of claims 145-161, wherein a first viral vector comprises said nucleic acid encoding said engineered nuclease.

169. The method of claim 168, wherein a first recombinant AAV vector comprises said nucleic acid encoding said engineered nuclease.

170. The method of claim 168 or 169, wherein a second viral vector comprises said template nucleic acid.

171. The method of claim 170, wherein a second recombinant AAV vector comprises said template nucleic acid.

172. The method of any one of claims 145-161 and 168-171, wherein said method comprises contacting said eukaryotic cell with only two populations of viral vectors, wherein a first population of viral vectors comprises said nucleic acid encoding said engineered nuclease, and wherein a second population of viral vectors comprises said template nucleic acid.

173. The method of claim 172, wherein said eukaryotic cell is contacted with:
(a) a first population of recombinant AAV vectors comprising said nucleic acid encoding said engineered nuclease; and
(b) a second population of recombinant AAV vectors comprising said template nucleic acid.

174. The method of any one of claims 145-173, wherein said engineered nuclease is an engineered meganuclease, a TALEN, a compact TALEN, a megaTAL, a zinc finger nuclease (ZFN), or a CRISPR system nuclease.

175. The method of claim 174, wherein said engineered nuclease is an engineered meganuclease having specificity for a recognition sequence within intron 1 of said transferrin gene.

176. The method of claim 175, wherein said meganuclease recognition sequence comprises SEQ ID NO: 19 or 21.

177. The method of claim 176, wherein said engineered nuclease is said engineered meganuclease of any one of claims 1-44.

178. The method of claim 174, wherein said engineered nuclease is a TALEN having specificity for a TALEN recognition sequence within intron 1 of said transferrin gene.

179. The method of claim 174, wherein said engineered nuclease is a zinc finger nuclease having specificity for a zinc finger nuclease recognition sequence within intron 1 of said transferrin gene.

180. The method of claim 174, wherein said engineered nuclease is a CRISPR system nuclease having specificity for a recognition sequence within intron 1 of said transferrin gene.

181. The method of any one of claims 145-180, wherein said eukaryotic cell is a mammalian cell.

182. The method of claim 181, wherein said mammalian cell is selected from a human cell, non-human primate cell, or a mouse cell.

183. The method of claim 181 or claim 182, wherein said mammalian cell is a hepatocyte.

184. The method of claim 183, wherein said hepatocyte is within the liver of a human, a non-human primate, or a mouse.

185. A method for producing a genetically-modified cell in a mammalian subject, wherein said genetically-modified cell comprises a modified transferrin gene, said method comprising delivering to a target cell in said subject:
(a) a nucleic acid encoding an engineered nuclease having specificity for a recognition sequence within intron 1 of a transferrin gene, wherein said engineered nuclease is expressed in said target cell; and
(b) a template nucleic acid comprising an exogenous nucleic acid molecule, wherein said exogenous nucleic acid molecule comprises, from 5′ to 3′:
(i) an exogenous splice acceptor sequence;
(ii) a first nucleic acid sequence encoding a C-terminal fragment of a signal peptide;
(iii) a second nucleic acid sequence encoding a polypeptide of interest; and
(iv) a polyA signal;
wherein said engineered nuclease produces a cleavage site at said recognition sequence within intron 1 of said transferrin gene, and wherein said exogenous nucleic acid molecule is inserted into intron 1 of said transferrin gene at said cleavage site, thereby generating a modified transferrin gene in said target cell in said subject.

186. The method of claim 185, wherein said first nucleic acid sequence is capable of being joined directly to the 3′ end of SEQ ID NO: 8 to generate a coding sequence for a transferrin signal peptide having at least 80% sequence identity to SEQ ID NO: 7.

187. The method of claim 185 or claim 186, wherein said first nucleic acid sequence has at least 80% sequence identity to SEQ ID NO: 9.

188. The method of any one of claims 185-187, wherein said first nucleic acid sequence comprises SEQ ID NO: 9.

189. The method of any one of claims 185-188, wherein said exogenous nucleic acid molecule further comprises a 5′ homology arm which is positioned 5′ upstream of said exogenous splice acceptor sequence, and a 3′ homology arm which is positioned 3′ downstream of said polyA signal, wherein said 5′ homology arm and said 3′ homology arm are homologous to sequences flanking said cleavage site, and wherein said exogenous nucleic acid molecule is inserted into said cleavage site by homologous recombination.

190. The method of claim 189, wherein upon generation of said modified transferrin gene, the endogenous promoter of said transferrin gene is operably linked to said exogenous nucleic acid molecule.

191. The method of any one of claims 185-190, wherein said endogenous promoter of said transferrin gene drives expression of said exogenous nucleic acid molecule.

192. The method of any one of claims 185-191, wherein said genetically-modified cell expresses a polypeptide comprising:
(a) a transferrin signal peptide having at least 80% sequence identity to SEQ ID NO: 7; and
(b) said polypeptide of interest;
wherein said polypeptide of interest is secreted by said genetically-modified cell.

193. The method of any one of claims 185-192, wherein said exogenous splice acceptor sequence has at least 80% sequence identity to SEQ ID NO: 10.

194. The method of any one of claims 185-193, wherein said exogenous splice acceptor sequence comprises SEQ ID NO: 10.

195. The method of any one of claims 185-192, wherein said exogenous splice acceptor sequence is not derived from intron 1 of said transferrin gene.

196. The method of any one of claims 185-195, wherein said exogenous nucleic acid molecule comprises, from 5′ to 3′:
(a) said exogenous splice acceptor sequence;
(b) said first nucleic acid sequence;
(c) a 2A sequence or IRES sequence;
(d) a third nucleic acid sequence encoding a signal peptide;
(e) said second nucleic acid sequence; and
(f) said polyA signal.

197. The method of claim 196, wherein said signal peptide encoded by said third nucleic acid sequence comprises an amino acid sequence having at least 80% sequence identity to SEQ ID NO: 7.

198. The method of claim 197, wherein said signal peptide encoded by said third nucleic acid sequence comprises an amino acid sequence of SEQ ID NO: 7.

199. The method of any one of claims 185-198, wherein said polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

200. The method of any one of claims 185-199, wherein said polyA signal comprises a nucleic acid sequence having at least 80% sequence identity to SEQ ID NO: 34.

201. The method of any one of claims 185-200, wherein said polyA signal comprises a nucleic acid sequence of SEQ ID NO: 34.

202. The method of any one of claims 185-201, wherein said nucleic acid encoding said engineered nuclease is an mRNA.

203. The method of claim 202, wherein said mRNA is packaged within a lipid nanoparticle.

204. The method of any one of claims 185-203, wherein a viral vector comprises said template nucleic acid.

205. The method of claim 204, wherein a recombinant AAV vector comprises said template nucleic acid.

206. The method of any one of claims 185-205, said method comprising delivering to said target cell:
(a) a lipid nanoparticle comprising an mRNA encoding said engineered nuclease; and
(b) a recombinant AAV vector comprising said template nucleic acid.

207. The method of claim 206, said method comprising delivering to said target cell:
(a) only one population of lipid nanoparticles comprising said mRNA encoding said engineered nuclease; and
(b) only one population of recombinant AAV vectors comprising said template nucleic acid.

208. The method of any one of claims 185-201, wherein a first viral vector comprises said nucleic acid encoding said engineered nuclease.

209. The method of claim 208, wherein a first recombinant AAV vector comprises said nucleic acid encoding said engineered nuclease.

210. The method of claim 208 or 209, wherein a second viral vector comprises said template nucleic acid.

211. The method of claim 210, wherein a second recombinant AAV vector comprises said template nucleic acid.

212. The method of any one of claims 185-201 and 208-211, said method comprising delivering only two populations of viral vectors to said target cell, wherein a first population of viral vectors comprises said nucleic acid encoding said engineered nuclease, and wherein a second population of viral vectors comprises said template nucleic acid.

213. The method of claim 212, said method comprising delivering to said target cell:
(a) a first population of recombinant AAV vectors comprising said nucleic acid encoding said engineered nuclease; and
(b) a second population of recombinant AAV vectors comprising said template nucleic acid.

214. The method of any one of claims 185-213, wherein said engineered nuclease cleavage site is generated by an engineered meganuclease, a TALEN, a compact TALEN, a megaTAL a zinc finger nuclease (ZFN), or a CRISPR system nuclease.

215. The method of claim 214, wherein said engineered nuclease is an engineered meganuclease having specificity for a recognition sequence within intron 1 of said transferrin gene.

216. The method of claim 215, wherein said meganuclease recognition sequence comprises SEQ ID NO: 19 or 21.

217. The method of claim 216, wherein said engineered nuclease is said engineered meganuclease of any one of claims 1-44.

218. The method of claim 214, wherein said engineered nuclease is a TALEN having specificity for a TALEN recognition sequence within intron 1 of said transferrin gene.

219. The method of claim 214, wherein said engineered nuclease is a zinc finger nuclease having specificity for a zinc finger nuclease recognition sequence within intron 1 of said transferrin gene.

220. The method of claim 214, wherein said engineered nuclease is a CRISPR system nuclease having specificity for a recognition sequence within intron 1 of said transferrin gene.

221. The method of any one of claims 185-220, wherein said mammalian subject is selected from a human, a non-human primate, or a mouse.

222. The method of any one of claims 185-221, wherein said target cell is a hepatocyte.

223. The method of claim 222, wherein said hepatocyte is within the liver of a human, a non-human primate, or a mouse.

224. A method for treating a disease in a subject in need thereof, said method comprising administering to said subject an effective amount of said pharmaceutical composition of any one of claims 111-144.

225. The method of claim 224, wherein said engineered nuclease produces a cleavage site at a recognition sequence within intron 1 of said transferrin gene, and wherein said exogenous nucleic acid molecule is inserted into intron 1 of said transferrin gene at said cleavage site, thereby generating a modified transferrin gene in said target cell in said subject.

226. The method of claim 224 or claim 225, wherein said method is effective to generate in said subject a genetically-modified target cell in vivo comprising a modified transferrin gene, wherein said modified transferrin gene comprises said exogenous nucleic acid molecule inserted within intron 1 of said transferrin gene.

227. The method of any one of claim 225 or 226, wherein upon generation of said modified transferrin gene, the endogenous promoter of said transferrin gene is operably linked to said exogenous nucleic acid molecule.

228. The method of any one of claims 225-227, wherein said endogenous promoter of said transferrin gene drives expression of said exogenous nucleic acid molecule.

229. The method of any one of claims 225-228, wherein said genetically-modified target cell expresses a polypeptide comprising:
(a) a transferrin signal peptide having at least 80% sequence identity to SEQ ID NO: 7; and
(b) said polypeptide of interest;
wherein said polypeptide of interest is secreted by said genetically-modified target cell.

230. The method of any one of claims 224-229, wherein said polypeptide of interest is acid alpha-glucosidase (GAA), alpha-galactosidase, glucosylceramidase beta, iduronate-2-sulfatase, arylsulfatase B, N-acetylgalactosamine-6-sulfatase, lysosomal acid lipase, alpha-1-antitrypsin, adenosine deaminase, or alpha-L-iduronidase.

231. The method of any one of claims 224-230, wherein said disease is Pompe disease, Fabry disease, Gaucher disease, Hunter syndrome, Marateaux-Lamy syndrome, Marquio A syndrome, lysosomal acid lipase deficiency, alpha-1-antitrypsin deficiency, adenosine deaminase deficiency, or Hurler syndrome.

232. The method of any one of claims 224-231, wherein said method is effective to treat said disease.

233. The method of any one of claims 224-232, wherein the method is effective to produce levels of said polypeptide of interest in said subject that are therapeutically beneficial or curative for said disease.

234. The engineered nuclease of any one of claims 1-44, for use as a medicament.

235. The engineered nuclease for use according to claim 234, wherein said medicament is useful for treating a disease in a subject in need thereof, such as a subject having Pompe disease, Fabry disease, Gaucher disease, Hunter syndrome, Marateaux-Lamy syndrome, Marquio A syndrome, lysosomal acid lipase deficiency, alpha-1-antitrypsin deficiency, adenosine deaminase deficiency, or Hurler syndrome.

236. The engineered nuclease of any one of claims 1-44, for use in manufacturing a medicament for treating a disease in a subject in need thereof, such as a subject having Pompe disease, Fabry disease, Gaucher disease, Hunter syndrome, Marateaux-Lamy syndrome, Marquio A syndrome, lysosomal acid lipase deficiency, alpha-1-antitrypsin deficiency, adenosine deaminase deficiency, or Hurler syndrome.


SEQUENCE LISTING (62 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 9 aa, Chlamydomonas reinhardtii
LAGLIDADG

SEQ ID NO: 3 - DNA, 43 bp, Homo sapiens
atgaggctcgccgtgggagccctgctggtctgcgccgtcctgg

SEQ ID NO: 4 - DNA, 1928 bp, Homo sapiens
gtgagtgcgggcacggggtagcaccgcagagtcgctggcccgcgcgttccctgcaacccgggcggccaccgcgcagcctgcatgcactccgcgctcaggctggaagcctgggtgtctgggtgcctttccattgcctctctacgccccacccctggcctttgcttgggcttctagacacctttcactgcactcactgtcttctccccctcctcctctttgtccagtaaatcttgcagcagtgaaatacaataaatcagagcacgtctaaccaggttatcaaaaccctttgctgctgcctcttgtcaagtccgaactccttagcatggcattcaaggcctcccgggtctgaccattcccctttcagaggctgatgtcccctgggtgccctgggagctgtagatgtcctgccaaggaagcggtgccatcgagcggtcagagcatggggtttgaagccagacttcttggatccaagtcctggccggctcctcaccagggagggggtcatcacagcacttgcctgggagggtcaaatgagggtcagcgaggtggcagatgctgagtaccagtggcagatgttgcacacatcctgctatggggcagtggctgacgccacactccccgacctcctgaggtggggcccccttcatactctaacttctgcctgccattcatggggcatttgtcacactgttggggcttccacgtttagttttctctccccataagctagcaattccttgagagaggaccccacgttacttgcctttatccccgcacagagcacttcacaggctgtgcaaggtaatgctccactgaggacacattctcgcctatgggaactctggaggcagggctttgtggaggggctgccacaggcttatgttgcctcctaggatttcccatgggccaagagggaaaatgggggtcgctggggtggccatccctggtggcccctcctcatgcatcctggagagctgtgggcctcctctccacaggctttgtggatcttcttcagagccagtgagtcagccttgctgtggtggcccacaaggagtggagatgacagagggccttggggacagggcaaaagctcatgtgatagggatgctgtctctgcaaccacatggattttaatagttacccatggctggcttcagctttttttgtgaatggcagccaaaaagtgtggtgaaaatggagggatagttcagtggagacagaggggagactgttgaatttgtggagtttttagttcttcctaaacagagagcaacagacaagagttacagacaaattaattcttttcgttaatttaccctcaactactggccaagaaggggtgtaactcagaatgttttcttcttttgtgccctgagtagcagacccagagtctggagacccagattctaggcctggcttggccaacgacaagcagggtgaccttgggttaaaaaaaaaaaaaggcatcattctctgagtctcactccctcttctcaaaaagatggcaattcctcccccgctggttttccatagttgaggtagcaagccaatgtgttggggtagctgaaaacgccctgtgcatactgaggcttatgttccatggggggccaggggccaggacagtgatgggaggagacaaggcggatacagaggagcagattgtcatctccagctgggaggtgatggccatgcctgcacccctctggccagcagagggtggtcagtaggaaactgggaggccattagggcaaccttctattggctcagactcagaatgcagtagaacttgtgccctgtagtgttcatggacaggagtgagaggaggacaggactgaggggatgtggctgtcaaggcctttctaggggcgatgctgtctctccctcagcatagggagtgggcccttccacctctggcctctctcccccag

SEQ ID NO: 5 - DNA, 173 bp, Homo sapiens
ggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcg

SEQ ID NO: 6 - DNA, 57 bp, Homo sapiens
atgaggctcgccgtgggagccctgctggtctgcgccgtcctggggctgtgtctggct

SEQ ID NO: 7 - protein, 19 aa, Homo sapiens
MRLAVGALLVCAVLGLCLA

SEQ ID NO: 8 - DNA, 43 bp, Homo sapiens; identical to SEQ ID NO: 3

SEQ ID NO: 9 - DNA, 14 bp, Homo sapiens
ggctgtgtctggct

SEQ ID NO: 10 - DNA, 49 bp, Homo sapiens
ccctcagcatagggagtgggcccttccacctctggcctctctcccccag

SEQ ID NO: 11 - DNA, 43 bp, Mus musculus
atgaggctcaccgtgggtgccctgctggcctgcgctgccctgg

SEQ ID NO: 12 - DNA, 2105 bp, Mus musculus
gtgagtgtgcgtgcaggggatgactggctggctatgtatgcaacccttaggagctaaagctggaagtcccagcttccagccccttgcttcttctctgtggtatcttcatcttccatagcgtaggaacaacgtggatccacatgcatctggggctctgattcagtctctacctttcactatcctcatcacgctgtctcccctatgtccagtaatttgaagatatggtatacgcgatgttagaaaacttttaaccagtttacccaggcgctatcccggggcctcagtatagtccaaacagcatggtgtggtagtcacaacttcctgagatctaatcctgctatctctcctttgtctgatgcctccttggatgcctttggcgctggtggcttagactagggtgtgggatttgaagccagaattctggggttcaaatcatacctgtatttctcctgcgacgggtcttgcttgttaccgcacatgcctgggagggtcaggtgagggtgaggtcacaggtcccagtcctgcatcctgctgactcctcaccagagggggggggggtctcaaagacccaaatgcctgctccagttggactgtagttagcatttgtcacagtgttggggctgtctagtttgagtgatttgtcatccctaacaactgtcagcctgaaacaggcttccgtgcttgttagctcaggacaccttgtgtggctatgcacagtgacaccggaccaaggtcaaaatttacaagttagacgaactagtttgaagaggctagagaggtcgtctatccctctggccacggtgaactaaagggaaggagctggggacgatcagtctccctgttgtccttcctcatccatcctgaggtccattggcctcacccaccccgatccatgcttagtgatttgtgggcttgctgagtcagtaagtcagccctgctgaattgcccgaaagggaagggatgtgacaagagctaggagtgccaggtcccacagacggcatctctccatcgtgtggatttcagtggtgacccagctggggagatggtacaactgggcagatgcttagcatgcaaacaccaggacctaaattcagaaccccagcaaccatgtaaaaagctgggtaccgcagtgccctggggaagcagacacaggaagatcccagggaacccacagtgccctggggaagcaaacacaggaagatcccagggactttctggccagacaatctagtggaaccagcaaggctcagattcagtgaaagacactgtctccaaaaaaatcaggcagagagtaatagcgggagacaccgcacgttgccctttgtcccccacccccaccccccatgtgttcccatctgcacatgcacacaccacatgggagcccgattatataagcacatacacacatcgtgatacacgtaattgtcttcagctgattctctaaatggcggcccaaagggggtaaagcatccagggaaggtttggtagaaacagaagcctgctgcatagaagggagttcctagccttttctcaacagaagacagctgaagagtatcacaggtgaactcgtgatcaaactgtcgctcagagagacgcaaagcttctaagatcgttatcttcccctggggcctggccatcagacctagtgtctgggctgctaacaaacatgatgacgtaggacaaacctgcttcctcccccaaggccagctttctcatttctaaaagacggtatgcctttcctagagttggtgtctagagccttgtctgaaggtggctgaggatggtgtttgcccactgaagttcacattttgatgtggaccttcagccaaggcaaaagaagagcaggttgtcaccttcagctaccccagcatctctctaagcagcccagggttaggtgggaacatggcagaccgttggggcgaacggattcatctgttcccaggatcggagtgcagcagagccctgtgctctgtcatgactgccaccagggatgagaggagggtggaacagaggagaggtggccattaagacccttcgtaaagtggtgcgttctctccccaggcacggggagcgggtcctttatgcctccgccttctctcctgcag

SEQ ID NO: 13 - DNA, 173 bp, Mus musculus
ggctgtgtctggctgtccctgacaaaacggtcaaatggtgcgcagtgtcagagcacgagaataccaagtgcatcagcttccgtgaccacatgaagaccgtccttccgcctgatggcccccggcttgcctgtgtgaagaaaacctcctatccggattgcatcaaggccatttct

SEQ ID NO: 14 - DNA, 57 bp, Mus musculus
atgaggctcaccgtgggtgccctgctggcctgcgctgccctggggctgtgtctggct

SEQ ID NO: 15 - protein, 19 aa, Mus musculus
MRLTVGALLACAALGLCLA

SEQ ID NO: 16 - DNA, 43 bp, Mus musculus; identical to SEQ ID NO: 11

SEQ ID NO: 17 - DNA, 14 bp, Mus musculus
ggctgtgtctggct

SEQ ID NO: 18 - DNA, 51 bp, Mus musculus
tccccaggcacggggagcgggtcctttatgcctccgccttctctcctgcag

SEQ ID NO: 19 - DNA, 22 bp, Homo sapiens
tcacaggctgtgcaaggtaatg

SEQ ID NO: 20 - DNA, 22 bp, Homo sapiens
agtgtccgacacgttccattac

SEQ ID NO: 21 - DNA, 22 bp, Mus musculus
ggggagatggtacaactgggca

SEQ ID NO: 22 - DNA, 22 bp, Mus musculus
cccctctaccatgttgacccgt

SEQ ID NO: 23 - protein, 354 aa, Artificial sequence
  Synthetic polypeptide
MNTKYNKEFLLYLAGFVDGDGSICASILPAQNRKFKHALLIRFKVSQKTQRRWFLDKLVDEIGVGYVHDLGSVSEYTLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYACIRPDQRAKFKHHLQLNFNVTQKTQRRWFLDKLVDEIGVGYVRDTGSVSQYLLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 24 - protein, 147 aa, Artificial sequence
  Synthetic polypeptide
KEFLLYLAGFVDGDGSIYACIRPDQRAKFKHHLQLNFNVTQKTQRRWFLDKLVDEIGVGYVRDTGSVSQYLLSEIKPLHNFLTQLQPFLKLKQKQANLVLKIIEQLPSAKESPDKFLEVCTWVDQIAALNDSKTRKTTSETVRAVLD

SEQ ID NO: 25 - protein, 147 aa, Artificial sequence
  Synthetic polypeptide
KEFLLYLAGFVDGDGSICASILPAQNRKFKHALLIRFKVSQKTQRRWFLDKLVDEIGVGYVHDLGSVSEYTLSEIKPLHNFLTQLQPFLKLKQKQANLVLKIIEQLPSAKESPDKFLEVCTWVDQIAALNDSKTRKTTSETVRAVLD

SEQ ID NO: 26 - protein, 354 aa, Artificial sequence
  Synthetic polypeptide
MNTKYNKEFLLYLAGFVDGDGSIWASIRPSQHRKFKHELRLCFQVTQKTQRRWFLDKLVDEIGVGYVADKGSVSHYSLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSISACIAPCQHRKFKHKLRLFFDVWQKTQRRWFLDKLVDEIGVGYVGDCGSVSRYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 27 - protein, 147 aa, Artificial sequence
  Synthetic polypeptide
KEFLLYLAGFVDGDGSISACIAPCQHRKFKHKLRLFFDVWQKTQRRWFLDKLVDEIGVGYVGDCGSVSRYRLSQIKPLHNFLTQLQPFLKLKQKQANLVLKIIEQLPSAKESPDKFLEVCTWVDQIAALNDSKTRKTTSETVRAVLD

SEQ ID NO: 28 - protein, 147 aa, Artificial sequence
  Synthetic polypeptide
KEFLLYLAGFVDGDGSIWASIRPSQHRKFKHELRLCFQVTQKTQRRWFLDKLVDEIGVGYVADKGSVSHYSLSEIKPLHNFLTQLQPFLKLKQKQANLVLKIIEQLPSAKESPDKFLEVCTWVDQIAALNDSKTRKTTSETVRAVLD

SEQ ID NO: 29 - DNA, 581 bp, Artificial sequence
  Synthetic polynucleotide
cgcccctctccctcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggcc

SEQ ID NO: 30 - DNA, 54 bp, Artificial sequence
  Synthetic polynucleotide
gagggcagaggaagtctgctaacatgcggtgacgtcgaggagaatcctggacct

SEQ ID NO: 31 - DNA, 57 bp, Artificial sequence
  Synthetic polynucleotide
gctactaacttcagcctgctgaagcaggctggagacgtggaggagaaccctggacct

SEQ ID NO: 32 - DNA, 60 bp, Artificial sequence
  Synthetic polynucleotide
cagtgtactaattatgctctcttgaaattggctggagatgttgagagcaaccctggacct

SEQ ID NO: 33 - DNA, 66 bp, Artificial sequence
  Synthetic polynucleotide
gtgaaacagactttgaattttgaccttctcaagttggcgggagacgtggagtccaaccctggacct

SEQ ID NO: 34 - DNA, 122 bp, Artificial sequence
  Synthetic polynucleotide
aacttgtttattgcagcttataatggttacaaataaagcaatagcatcacaaatttcacaaataaagcatttttttcactgcattctagttgtggtttgtccaaactcatcaatgtatctta

SEQ ID NO: 35 - protein, 4 aa, Homo sapiens
LCLA

SEQ ID NO: 36 - DNA, 22 bp, Artificial sequence
  Synthetic polynucleotide
cgtggatccacatgcatctggg

SEQ ID NO: 37 - DNA, 20 bp, Artificial sequence
  Synthetic polynucleotide
cccagtgcctctgcagcttc

SEQ ID NO: 38 - DNA, 18 bp, Artificial sequence
  Synthetic polynucleotide
ctgagtcagtaagtcagc

SEQ ID NO: 39 - DNA, 20 bp, Artificial sequence
  Synthetic polynucleotide
gtctttcactgaatctgagc

SEQ ID NO: 40 - DNA, 17 bp, Artificial sequence
  Synthetic polynucleotide
cccagttgtaccatctc

SEQ ID NO: 41 - DNA, 20 bp, Artificial sequence
  Synthetic polynucleotide
tcatacctgtatttctcctg

SEQ ID NO: 42 - DNA, 18 bp, Artificial sequence
  Synthetic polynucleotide
gctggagtttcttagcag

SEQ ID NO: 43 - DNA, 22 bp, Artificial sequence
  Synthetic polynucleotide
cacaggtcccagtcctgcatcc

SEQ ID NO: 44 - DNA, 20 bp, Artificial sequence
  Synthetic polynucleotide
ctagacctttcagttcatgc

SEQ ID NO: 45 - DNA, 20 bp, Artificial sequence
  Synthetic polynucleotide
tgtccagatgtcaaagagat

SEQ ID NO: 46 - DNA, 21 bp, Artificial sequence
  Synthetic polynucleotide
ttcagcctgccacaggctcac

SEQ ID NO: 47 - DNA, 20 bp, Artificial sequence
  Synthetic polynucleotide
tgtctttaactggtcatagg

SEQ ID NO: 48 - DNA, 20 bp, Artificial sequence
  Synthetic polynucleotide
gaacaagaactgaagagaac

SEQ ID NO: 49 - DNA, 21 bp, Artificial sequence
  Synthetic polynucleotide
ttcagcctgccacaggctcac

SEQ ID NO: 50 - protein, 42 aa, Artificial sequence
  Synthetic polypeptide
SLPGSVGGLSPSQASSAASSASSSPGSGISEALRAGAGSGTG

SEQ ID NO: 51 - DNA, 2974 bp, Artificial sequence
  Synthetic polynucleotide
ggggagatggtacaactgggcatgttttaaggggggggtctcaaagacccaaatgcctgctccagttggactgtagttagcatttgtcacagtgttggggctgtctagtttgagtgatttgtcatccctaacaactgtcagcctgaaacaggcttccgtgcttgttagctcaggacaccttgtgtggctatgcacagtgacaccggaccaaggtcaaaatttacaagttagacgaactagtttgaagaggctagagaggtcgtctatccctctggccacggtgaactaaagggaaggagctggggacgctcagtctccctgttgtccttcctcatccatcctgaggtccattggcctcacccaccccgatccatgcttagtgatttgtggacttgctgagtcagtaagtcagccctgctgaattgcccgaaagggaagggatgggacaagagctaggagtgccaggtcccacagacggcatctctccatcgtgtggatttcagtggtgacccagctggggagatggtacaccggtcctaggggtggaacagaggagaggtggccattaagacccttcgtaaagtggtgcgttctctccccaggcacggggagcgggtcctttatgcctccgccttctctcctgcagggctgtgtctggctggaagcggagagggcagaggaagtctgctaacatgcggtgacgtcgaggagaatcctggacctaacaggctcgccgtgggagccctgctggtctgcgccgtcctggggctgtgtctggctatcattcccgtggaggaagagaacccagacttctggaatcgggaagctgcagaggcactgggagctgctaagaaactccagcctgcacaaaccgccgctaagaacctgatcatcttcctcggagatggcatgggagtcagcacagtgactgccgcaaggatcctgaaaggccagaagaaagacaagctcggacccgaaattccactggctatggatcggttcccttacgtcgcactctccaagacctacaacgtggacaagcacgtccccgatagcggagctacagctactgcatacctgtgcggagtgaaaggcaacttccagaccatcggactctccgcagctgcaaggttcaatcagtgtaacacaactcggggcaatgaggtcattagcgtgatgaacagggccaagaaagctggaaagtccgtcggcgtggtcaccacaactcgggtgcaacatgcaagcccagcaggaacctatgctcacacagtcaataggaactggtactccgacgcagatgtgcctgccagcgctcggcaggaaggctgccaagacatcgcaactcagctgatctccaacatggatatcgacgtcattctcggaggcggaaggaagtacatgttccggatgggaaccccagatccagagtaccctgacgattatagccaaggaggcacaaggctggacggaaagaacctcgtgcaggaatggctggccaaacggcaaggcgctaggtacgtctggaatcggactgagctcatgcaggcatccctggatcccagcgtgacccatctcatgggactgttcgaaccaggcgacatgaagtatgagatccacagggattccacactcgaccctagcctgatggaaatgactgaggccgctctccggctgctctccaggaaccctaggggatttttcctgtttgtcgaaggcggaaggattgatcatggccaccatgagagccgggcatacagggccctcaccgagaccatcatgttcgacgatgctattgagcgggcaggacaactgacttccgaagaggacaccctcagcctggtgacagccgatcactcccatgtctttagcttcggcggatatccactcaggggctccagcatctttggactggctcctggcaaagcacgggacaggaaggcctacactgtgctcctgtatggaaatggccccggatacgtcctcaaagatggagctaggccagacgtgaccgaatccgagagcggatcccctgaatataggcagcaaagcgcagtccccctggatgaggaaacacacgctggagaggacgtggctgtcttcgcacggggaccacaggcccatctcgtgcacggcgtccaagaacagactttcatcgctcacgtgatggcattcgccgcttgtctggagccttacaccgcatgcgatctcgcacctccagctggaacaactgacgcagcccaccctggctattccagggtcggagcagctggccggttcgagcaaacctgagtcgacaacttgtttattgcagcttataatggttacaaataaagcaatagcatcacaaatttcacaaataaagcatttttttcactgcattctagttgtggtttgtccaaactcatcaatgtatcttaagatctgtacaactgggcagatgcttagcatgcaaacaccaggacctaaattcagaaccccagcaaccatgtaaaaagctgggtaccgcagtgccctggggaagcagacacaggaagatcccagggaacccacagtgccctggggaagcaaacacaggaagatcccagggactttctggccagacaatctagtggaaccagcaaggctcagattcagtgaaagacactgtctccaaaaaaatcaggcagagagtaatagcgggagacaccgcacgttgccctttgtcccccacccccaccccccatgtgttcccatctgcacatgcacacaccacatgggagcccgattatataagcacatacacacatcgtgatacacgtaattgtcttcagctgattctctaaatggcggcccaaagggggtaaagcatccagggaaggtttggtagaaacagaagcctgctgcatagaagggagttcctagccttttctcaacagaagacagctgaagagtatcacaggtgaactcccgggcggggagatggtacaactgggca

SEQ ID NO: 52 - DNA, 4518 bp, Artificial sequence
  Synthetic polynucleotide
ggggagatggtacaactgggcatgttttaaggggggggtctcaaagacccaaatgcctgctccagttggactgtagttagcatttgtcacagtgttggggctgtctagtttgagtgatttgtcatccctaacaactgtcagcctgaaacaggcttccgtgcttgttagctcaggacaccttgtgtggctatgcacagtgacaccggaccaaggtcaaaatttacaagttagacgaactagtttgaagaggctagagaggtcgtctatccctctggccacggtgaactaaagggaaggagctggggacgctcagtctccctgttgtccttcctcatccatcctgaggtccattggcctcacccaccccgatccatgcttagtgatttgtggacttgctgagtcagtaagtcagccctgctgaattgcccgaaagggaagggatgggacaagagctaggagtgccaggtcccacagacggcatctctccatcgtgtggatttcagtggtgacccagctggggagatggtacaccggtcctaggggtggaacagaggagaggtggccattaagacccttcgtaaagtggtgcgttctctccccaggcacggggagcgggtcctttatgcctccgccttctctcctgcagggctgtgtctggctatcattcccgtggaggaagagaacccagacttctggaatcgggaagctgcagaggcactgggagctgctaagaaactccagcctgcacaaaccgccgctaagaacctgatcatcttcctcggagatggcatgggagtcagcacagtgactgccgcaaggatcctgaaaggccagaagaaagacaagctcggacccgaaattccactggctatggatcggttcccttacgtcgcactctccaagacctacaacgtggacaagcacgtccccgatagcggagctacagctactgcatacctgtgcggagtgaaaggcaacttccagaccatcggactctccgcagctgcaaggttcaatcagtgtaacacaactcggggcaatgaggtcattagcgtgatgaacagggccaagaaagctggaaagtccgtcggcgtggtcaccacaactcgggtgcaacatgcaagcccagcaggaacctatgctcacacagtcaataggaactggtactccgacgcagatgtgcctgccagcgctcggcaggaaggctgccaagacatcgcaactcagctgatctccaacatggatatcgacgtcattctcggaggcggaaggaagtacatgttccggatgggaaccccagatccagagtaccctgacgattatagccaaggaggcacaaggctggacggaaagaacctcgtgcaggaatggctggccaaacggcaaggcgctaggtacgtctggaatcggactgagctcatgcaggcatccctggatcccagcgtgacccatctcatgggactgttcgaaccaggcgacatgaagtatgagatccacagggattccacactcgaccctagcctgatggaaatgactgaggccgctctccggctgctctccaggaaccctaggggatttttcctgtttgtcgaaggcggaaggattgatcatggccaccatgagagccgggcatacagggccctcaccgagaccatcatgttcgacgatgctattgagcgggcaggacaactgacttccgaagaggacaccctcagcctggtgacagccgatcactcccatgtctttagcttcggcggatatccactcaggggctccagcatctttggactggctcctggcaaagcacgggacaggaaggcctacactgtgctcctgtatggaaatggccccggatacgtcctcaaagatggagctaggccagacgtgaccgaatccgagagcggatcccctgaatataggcagcaaagcgcagtccccctggatgaggaaacacacgctggagaggacgtggctgtcttcgcacggggaccacaggcccatctcgtgcacggcgtccaagaacagactttcatcgctcacgtgatggcattcgccgcttgtctggagccttacaccgcatgcgatctcgcacctccagctggaacaactgacgcagcccaccctggctattccagggtcggagcagctggccggttcgagcaaacctgagtcgacaacttgtttattgcagcttataatggttacaaataaagcaatagcatcacaaatttcacaaataaagcatttttttcactgcattctagttgtggtttgtccaaactcatcaatgtatcttaagatctgtacaactgggcagatgcttagcatgcaaacaccaggacctaaattcagaaccccagcaaccatgtaaaaagctgggtaccgcagtgccctggggaagcagacacaggaagatcccagggaacccacagtgccctggggaagcaaacacaggaagatcccagggactttctggccagacaatctagtggaaccagcaaggctcagattcagtgaaagacactgtctccaaaaaaatcaggcagagagtaatagcgggagacaccgcacgttgccctttgtcccccacccccaccccccatgtgttcccatctgcacatgcacacaccacatgggagcccgattatataagcacatacacacatcgtgatacacgtaattgtcttcagctgattctctaaatggcggcccaaagggggtaaagcatccagggaaggtttggtagaaacagaagcctgctgcatagaagggagttcctagccttttctcaacagaagacagctgaagagtatcacaggtgaactcccgggcggggagatggtacaactgggcacccggggtttaaactgcagcctaggcgcgcctcaataatcaatgccaacatggctgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccgctggtaccttagggggacgacttcttcttctcggagagactgtctagaacggcgcggacggtttcggaagtggtcttgcgggtcttggagtcgttcagagcggcgatctggtccacccaggtgcacacctccaggaacttgtccggggattccttggcggagggcagctgctcgatgatcttcagcacgaggttggcctgcttctgcttgagcttcaggaagggctggagctgggtcaggaagttgtgcagaggcttgatctggctcagacggtaacgggagacgctgccacagtcccccacgtaacccacaccgatctcgtccaccagcttgtcgaggaaccaacggcgctgtgtcttctgccagacatcgaaaaagagcctcagcttgtgcttgaacttccgatgttgacacggagcgatacaggcactgatggagccgtccccgtcgacgaagcccgccaggtagagcaggaattccttgttgtatccagtgccggaacctgctccagctctgagtgcttcggagatccctgaacccgggcttgaggaagccgaggatgcggcgctggatgcctgagatggcgatagacctcccacggatcctggtaaactgtctagcacagcacgaacggtttcagaagttgttttacgcgtcttcgaatcattcagagctgcaatttgatccacccatgtacaaacttctaagaatttgtccggggattcttttgctgacggaagttgttcaataatcttcaaaaccaagtttgcttgcttttgctttagcttcagaaaaggttgcagttgtgtcaaaaaattatgcaaaggcttgatctcggacagagagtaatgggagacgctgcccttgtcagccacgtaacccacaccgatctcgtccaccagcttgtcgaggaaccaacggcgctgtgtcttctgcgtgacctggaaacagagacgcagctcgtgcttgaacttccgatgttgcgaaggacggatagaggcccagatggaaccgtcaccgtctacaaaccctgctaagtagagtaagaactcctgtggagagaaaggcaaagtggatgtcagtaagaccaataggtgcctatcagaaacgcaagagtcttctctgtctcgacaagccaagcttctattggcgccttaaacctgtcttgtaaccttgatacttaccttgttgtacttggtgttcatatgcaccttgcgcttcttcttcggtgccatggtggcaattgtgagcggccaacatggcggtcatattggacatgagccaatataaatacaaacaagaaccgcgacccaaatcccggctgcgacggaactagctgtgccacacccggcgcgtccttatataatcatcggcgttcaccgcccattctccgcccagccataaaaggcaactttcggaacggcgcacgctgattggctccgccctaactccgccc

SEQ ID NO: 53 - DNA, 3342 bp, Artificial sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggggggggtctcaaagacccaaatgcctgctccagttggactgtagttagcatttgtcacagtgttggggctgtctagtttgagtgatttgtcatccctaacaactgtcagcctgaaacaggcttccgtgcttgttagctcaggacaccttgtgtggctatgcacagtgacaccggaccaaggtcaaaatttacaagttagacgaactagtttgaagaggctagagaggtcgtctatccctctggccacggtgaactaaagggaaggagctggggacgctcagtctccctgttgtccttcctcatccatcctgaggtccattggcctcacccaccccgatccatgcttagtgatttgtggacttgctgagtcagtaagtcagccctgctgaattgcccgaaagggaagggatgggacaagagctaggagtgccaggtcccacagacggcatctctccatcgtgtggatttcagtggtgacccagctggggagatggtacaccggtcctaggggtggaacagaggagaggtggccattaagacccttcgtaaagtggtgcgttctctccccaggcacggggagcgggtcctttatgcctccgccttctctcctgcagggctgtgtctggctggaagcggagagggcagaggaagtctgctaacatgcggtgacgtcgaggagaatcctggacctaacaggctcgccgtgggagccctgctggtctgcgccgtcctggggctgtgtctggctatcattcccgtggaggaagagaacccagacttctggaatcgggaagctgcagaggcactgggagctgctaagaaactccagcctgcacaaaccgccgctaagaacctgatcatcttcctcggagatggcatgggagtcagcacagtgactgccgcaaggatcctgaaaggccagaagaaagacaagctcggacccgaaattccactggctatggatcggttcccttacgtcgcactctccaagacctacaacgtggacaagcacgtccccgatagcggagctacagctactgcatacctgtgcggagtgaaaggcaacttccagaccatcggactctccgcagctgcaaggttcaatcagtgtaacacaactcggggcaatgaggtcattagcgtgatgaacagggccaagaaagctggaaagtccgtcggcgtggtcaccacaactcgggtgcaacatgcaagcccagcaggaacctatgctcacacagtcaataggaactggtactccgacgcagatgtgcctgccagcgctcggcaggaaggctgccaagacatcgcaactcagctgatctccaacatggatatcgacgtcattctcggaggcggaaggaagtacatgttccggatgggaaccccagatccagagtaccctgacgattatagccaaggaggcacaaggctggacggaaagaacctcgtgcaggaatggctggccaaacggcaaggcgctaggtacgtctggaatcggactgagctcatgcaggcatccctggatcccagcgtgacccatctcatgggactgttcgaaccaggcgacatgaagtatgagatccacagggattccacactcgaccctagcctgatggaaatgactgaggccgctctccggctgctctccaggaaccctaggggatttttcctgtttgtcgaaggcggaaggattgatcatggccaccatgagagccgggcatacagggccctcaccgagaccatcatgttcgacgatgctattgagcgggcaggacaactgacttccgaagaggacaccctcagcctggtgacagccgatcactcccatgtctttagcttcggcggatatccactcaggggctccagcatctttggactggctcctggcaaagcacgggacaggaaggcctacactgtgctcctgtatggaaatggccccggatacgtcctcaaagatggagctaggccagacgtgaccgaatccgagagcggatcccctgaatataggcagcaaagcgcagtccccctggatgaggaaacacacgctggagaggacgtggctgtcttcgcacggggaccacaggcccatctcgtgcacggcgtccaagaacagactttcatcgctcacgtgatggcattcgccgcttgtctggagccttacaccgcatgcgatctcgcacctccagctggaacaactgacgcagcccaccctggctattccagggtcggagcagctggccggttcgagcaaacctgagtcgacaacttgtttattgcagcttataatggttacaaataaagcaatagcatcacaaatttcacaaataaagcatttttttcactgcattctagttgtggtttgtccaaactcatcaatgtatcttaagatctgtacaactgggcagatgcttagcatgcaaacaccaggacctaaattcagaaccccagcaaccatgtaaaaagctgggtaccgcagtgccctggggaagcagacacaggaagatcccagggaacccacagtgccctggggaagcaaacacaggaagatcccagggactttctggccagacaatctagtggaaccagcaaggctcagattcagtgaaagacactgtctccaaaaaaatcaggcagagagtaatagcgggagacaccgcacgttgccctttgtcccccacccccaccccccatgtgttcccatctgcacatgcacacaccacatgggagcccgattatataagcacatacacacatcgtgatacacgtaattgtcttcagctgattctctaaatggcggcccaaagggggtaaagcatccagggaaggtttggtagaaacagaagcctgctgcatagaagggagttcctagccttttctcaacagaagacagctgaagagtatcacaggtgaactctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 3456 bp, Artificial sequence
  Synthetic polynucleotide
ccttaattaggctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctaggaagatcggaattcgcccttaagctagcattggcgcgccggccggcctgcccagttgtaccatctcccctgttttcttaaggggggggtctcaaagacccaaatgcctgctccagttggactgtagttagcatttgtcacagtgttggggctgtctagtttgagtgatttgtcatccctaacaactgtcagcctgaaacaggcttccgtgcttgttagctcaggacaccttgtgtggctatgcacagtgacaccggaccaaggtcaaaatttacaagttagacgaactagtttgaagaggctagagaggtcgtctatccctctggccacggtgaactaaagggaaggagctggggacgctcagtctccctgttgtccttcctcatccatcctgaggtccattggcctcacccaccccgatccatgcttagtgatttgtggacttgctgagtcagtaagtcagccctgctgaattgcccgaaagggaagggatgggacaagagctaggagtgccaggtcccacagacggcatctctccatcgtgtggatttcagtggtgacccagctggggagatggtacaccggtcctaggggtggaacagaggagaggtggccattaagacccttcgtaaagtggtgcgttctctccccaggcacggggagcgggtcctttatgcctccgccttctctcctgcagggctgtgtctggctggaagcggagagggcagaggaagtctgctaacatgcggtgacgtcgaggagaatcctggacctaacaggctcgccgtgggagccctgctggtctgcgccgtcctggggctgtgtctggctatcattcccgtggaggaagagaacccagacttctggaatcgggaagctgcagaggcactgggagctgctaagaaactccagcctgcacaaaccgccgctaagaacctgatcatcttcctcggagatggcatgggagtcagcacagtgactgccgcaaggatcctgaaaggccagaagaaagacaagctcggacccgaaattccactggctatggatcggttcccttacgtcgcactctccaagacctacaacgtggacaagcacgtccccgatagcggagctacagctactgcatacctgtgcggagtgaaaggcaacttccagaccatcggactctccgcagctgcaaggttcaatcagtgtaacacaactcggggcaatgaggtcattagcgtgatgaacagggccaagaaagctggaaagtccgtcggcgtggtcaccacaactcgggtgcaacatgcaagcccagcaggaacctatgctcacacagtcaataggaactggtactccgacgcagatgtgcctgccagcgctcggcaggaaggctgccaagacatcgcaactcagctgatctccaacatggatatcgacgtcattctcggaggcggaaggaagtacatgttccggatgggaaccccagatccagagtaccctgacgattatagccaaggaggcacaaggctggacggaaagaacctcgtgcaggaatggctggccaaacggcaaggcgctaggtacgtctggaatcggactgagctcatgcaggcatccctggatcccagcgtgacccatctcatgggactgttcgaaccaggcgacatgaagtatgagatccacagggattccacactcgaccctagcctgatggaaatgactgaggccgctctccggctgctctccaggaaccctaggggatttttcctgtttgtcgaaggcggaaggattgatcatggccaccatgagagccgggcatacagggccctcaccgagaccatcatgttcgacgatgctattgagcgggcaggacaactgacttccgaagaggacaccctcagcctggtgacagccgatcactcccatgtctttagcttcggcggatatccactcaggggctccagcatctttggactggctcctggcaaagcacgggacaggaaggcctacactgtgctcctgtatggaaatggccccggatacgtcctcaaagatggagctaggccagacgtgaccgaatccgagagcggatcccctgaatataggcagcaaagcgcagtccccctggatgaggaaacacacgctggagaggacgtggctgtcttcgcacggggaccacaggcccatctcgtgcacggcgtccaagaacagactttcatcgctcacgtgatggcattcgccgcttgtctggagccttacaccgcatgcgatctcgcacctccagctggaacaactgacgcagcccaccctggctattccagggtcggagcagctggccggttcgagcaaacctgagtcgacaacttgtttattgcagcttataatggttacaaataaagcaatagcatcacaaatttcacaaataaagcatttttttcactgcattctagttgtggtttgtccaaactcatcaatgtatcttaagatctgtacaactgggcagatgcttagcatgcaaacaccaggacctaaattcagaaccccagcaaccatgtaaaaagctgggtaccgcagtgccctggggaagcagacacaggaagatcccagggaacccacagtgccctggggaagcaaacacaggaagatcccagggactttctggccagacaatctagtggaaccagcaaggctcagattcagtgaaagacactgtctccaaaaaaatcaggcagagagtaatagcgggagacaccgcacgttgccctttgtcccccacccccaccccccatgtgttcccatctgcacatgcacacaccacatgggagcccgattatataagcacatacacacatcgtgatacacgtaattgtcttcagctgattctctaaatggcggcccaaagggggtaaagcatccagggaaggtttggtagaaacagaagcctgctgcatagaagggagttcctagccttttctcaacagaagacagctgaagagtatcacaggtgaactcccgggctgcccagttgtaccatctcccccccggggtttaaactgcagcctctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 55 - DNA, 3233 bp, Artificial sequence
  Synthetic polynucleotide
ccttaattaggctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctaggaagatcggaattcgcccttaagctagcggggggggtctcaaagacccaaatgcctgctccagttggactgtagttagcatttgtcacagtgttggggctgtctagtttgagtgatttgtcatccctaacaactgtcagcctgaaacaggcttccgtgcttgttagctcaggacaccttgtgtggctatgcacagtgacaccggaccaaggtcaaaatttacaagttagacgaactagtttgaagaggctagagaggtcgtctatccctctggccacggtgaactaaagggaaggagctggggacgctcagtctccctgttgtccttcctcatccatcctgaggtccattggcctcacccaccccgatccatgcttagtgatttgtggacttgctgagtcagtaagtcagccctgctgaattgcccgaaagggaagggatgggacaagagctaggagtgccaggtcccacagacggcatctctccatcgtgtggatttcagtggtgacccagctggggagatggtacaccggtcctaggggtggaacagaggagaggtggccattaagacccttcgtaaagtggtgcgttctctccccaggcacggggagcgggtcctttatgcctccgccttctctcctgcagggctgtgtctggctatcattcccgtggaggaagagaacccagacttctggaatcgggaagctgcagaggcactgggagctgctaagaaactccagcctgcacaaaccgccgctaagaacctgatcatcttcctcggagatggcatgggagtcagcacagtgactgccgcaaggatcctgaaaggccagaagaaagacaagctcggacccgaaattccactggctatggatcggttcccttacgtcgcactctccaagacctacaacgtggacaagcacgtccccgatagcggagctacagctactgcatacctgtgcggagtgaaaggcaacttccagaccatcggactctccgcagctgcaaggttcaatcagtgtaacacaactcggggcaatgaggtcattagcgtgatgaacagggccaagaaagctggaaagtccgtcggcgtggtcaccacaactcgggtgcaacatgcaagcccagcaggaacctatgctcacacagtcaataggaactggtactccgacgcagatgtgcctgccagcgctcggcaggaaggctgccaagacatcgcaactcagctgatctccaacatggatatcgacgtcattctcggaggcggaaggaagtacatgttccggatgggaaccccagatccagagtaccctgacgattatagccaaggaggcacaaggctggacggaaagaacctcgtgcaggaatggctggccaaacggcaaggcgctaggtacgtctggaatcggactgagctcatgcaggcatccctggatcccagcgtgacccatctcatgggactgttcgaaccaggcgacatgaagtatgagatccacagggattccacactcgaccctagcctgatggaaatgactgaggccgctctccggctgctctccaggaaccctaggggatttttcctgtttgtcgaaggcggaaggattgatcatggccaccatgagagccgggcatacagggccctcaccgagaccatcatgttcgacgatgctattgagcgggcaggacaactgacttccgaagaggacaccctcagcctggtgacagccgatcactcccatgtctttagcttcggcggatatccactcaggggctccagcatctttggactggctcctggcaaagcacgggacaggaaggcctacactgtgctcctgtatggaaatggccccggatacgtcctcaaagatggagctaggccagacgtgaccgaatccgagagcggatcccctgaatataggcagcaaagcgcagtccccctggatgaggaaacacacgctggagaggacgtggctgtcttcgcacggggaccacaggcccatctcgtgcacggcgtccaagaacagactttcatcgctcacgtgatggcattcgccgcttgtctggagccttacaccgcatgcgatctcgcacctccagctggaacaactgacgcagcccaccctggctattccagggtcggagcagctggccggttcgagcaaacctgagtcgacaacttgtttattgcagcttataatggttacaaataaagcaatagcatcacaaatttcacaaataaagcatttttttcactgcattctagttgtggtttgtccaaactcatcaatgtatcttaagatctgtacaactgggcagatgcttagcatgcaaacaccaggacctaaattcagaaccccagcaaccatgtaaaaagctgggtaccgcagtgccctggggaagcagacacaggaagatcccagggaacccacagtgccctggggaagcaaacacaggaagatcccagggactttctggccagacaatctagtggaaccagcaaggctcagattcagtgaaagacactgtctccaaaaaaatcaggcagagagtaatagcgggagacaccgcacgttgccctttgtcccccacccccaccccccatgtgttcccatctgcacatgcacacaccacatgggagcccgattatataagcacatacacacatcgtgatacacgtaattgtcttcagctgattctctaaatggcggcccaaagggggtaaagcatccagggaaggtttggtagaaacagaagcctgctgcatagaagggagttcctagccttttctcaacagaagacagctgaagagtatcacaggtgaactctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - DNA, 3295 bp, Artificial sequence
  Synthetic polynucleotide
ccttaattaggctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctaggaagatcggaattcgcccttaagctagctgcccagttgtaccatctcccctgttttcttaaggggggggtctcaaagacccaaatgcctgctccagttggactgtagttagcatttgtcacagtgttggggctgtctagtttgagtgatttgtcatccctaacaactgtcagcctgaaacaggcttccgtgcttgttagctcaggacaccttgtgtggctatgcacagtgacaccggaccaaggtcaaaatttacaagttagacgaactagtttgaagaggctagagaggtcgtctatccctctggccacggtgaactaaagggaaggagctggggacgctcagtctccctgttgtccttcctcatccatcctgaggtccattggcctcacccaccccgatccatgcttagtgatttgtggacttgctgagtcagtaagtcagccctgctgaattgcccgaaagggaagggatgggacaagagctaggagtgccaggtcccacagacggcatctctccatcgtgtggatttcagtggtgacccagctggggagatggtacaccggtcctaggggtggaacagaggagaggtggccattaagacccttcgtaaagtggtgcgttctctccccaggcacggggagcgggtcctttatgcctccgccttctctcctgcagggctgtgtctggctatcattcccgtggaggaagagaacccagacttctggaatcgggaagctgcagaggcactgggagctgctaagaaactccagcctgcacaaaccgccgctaagaacctgatcatcttcctcggagatggcatgggagtcagcacagtgactgccgcaaggatcctgaaaggccagaagaaagacaagctcggacccgaaattccactggctatggatcggttcccttacgtcgcactctccaagacctacaacgtggacaagcacgtccccgatagcggagctacagctactgcatacctgtgcggagtgaaaggcaacttccagaccatcggactctccgcagctgcaaggttcaatcagtgtaacacaactcggggcaatgaggtcattagcgtgatgaacagggccaagaaagctggaaagtccgtcggcgtggtcaccacaactcgggtgcaacatgcaagcccagcaggaacctatgctcacacagtcaataggaactggtactccgacgcagatgtgcctgccagcgctcggcaggaaggctgccaagacatcgcaactcagctgatctccaacatggatatcgacgtcattctcggaggcggaaggaagtacatgttccggatgggaaccccagatccagagtaccctgacgattatagccaaggaggcacaaggctggacggaaagaacctcgtgcaggaatggctggccaaacggcaaggcgctaggtacgtctggaatcggactgagctcatgcaggcatccctggatcccagcgtgacccatctcatgggactgttcgaaccaggcgacatgaagtatgagatccacagggattccacactcgaccctagcctgatggaaatgactgaggccgctctccggctgctctccaggaaccctaggggatttttcctgtttgtcgaaggcggaaggattgatcatggccaccatgagagccgggcatacagggccctcaccgagaccatcatgttcgacgatgctattgagcgggcaggacaactgacttccgaagaggacaccctcagcctggtgacagccgatcactcccatgtctttagcttcggcggatatccactcaggggctccagcatctttggactggctcctggcaaagcacgggacaggaaggcctacactgtgctcctgtatggaaatggccccggatacgtcctcaaagatggagctaggccagacgtgaccgaatccgagagcggatcccctgaatataggcagcaaagcgcagtccccctggatgaggaaacacacgctggagaggacgtggctgtcttcgcacggggaccacaggcccatctcgtgcacggcgtccaagaacagactttcatcgctcacgtgatggcattcgccgcttgtctggagccttacaccgcatgcgatctcgcacctccagctggaacaactgacgcagcccaccctggctattccagggtcggagcagctggccggttcgagcaaacctgagtcgacaacttgtttattgcagcttataatggttacaaataaagcaatagcatcacaaatttcacaaataaagcatttttttcactgcattctagttgtggtttgtccaaactcatcaatgtatcttaagatctgtacaactgggcagatgcttagcatgcaaacaccaggacctaaattcagaaccccagcaaccatgtaaaaagctgggtaccgcagtgccctggggaagcagacacaggaagatcccagggaacccacagtgccctggggaagcaaacacaggaagatcccagggactttctggccagacaatctagtggaaccagcaaggctcagattcagtgaaagacactgtctccaaaaaaatcaggcagagagtaatagcgggagacaccgcacgttgccctttgtcccccacccccaccccccatgtgttcccatctgcacatgcacacaccacatgggagcccgattatataagcacatacacacatcgtgatacacgtaattgtcttcagctgattctctaaatggcggcccaaagggggtaaagcatccagggaaggtttggtagaaacagaagcctgctgcatagaagggagttcctagccttttctcaacagaagacagctgaagagtatcacaggtgaactcccgggctgcccagttgtaccatctccccctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 57 - DNA, 3155 bp, Artificial sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatctcctaggcgccaattgccaccatggcaccgaagaagaagcgcaaggtgcatatgaacaccaagtacaacaaggtaagtatcaaggttacaagacaggtttaaggcgccaatagaagcttggcttgtcgagacagagaagactcttgcgtttctgataggcacctattggtcttactgacatccactttgcctttctctccacaggagttcttactctacttagcagggtttgtagacggtgacggttccatctgggcctctatccgtccttcgcaacatcggaagttcaagcacgagctgcgtctctgtttccaggtcacgcagaagacacagcgccgttggttcctcgacaagctggtggacgagatcggtgtgggttacgtggctgacaagggcagcgtctcccattactctctgtccgagatcaagcctttgcataattttttgacacaactgcaaccttttctgaagctaaagcaaaagcaagcaaacttggttttgaagattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatcagtgcctgtatcgctccgtgtcaacatcggaagttcaagcacaagctgaggctctttttcgatgtctggcagaagacacagcgccgttggttcctcgacaagctggtggacgagatcggtgtgggttacgtgggggactgtggcagcgtctcccgttaccgtctgagccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtaccagcggccgatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 58 - DNA, 22 bp, Artificial sequence
  Synthetic polynucleotide
cattaccttgcacagcctgtga

SEQ ID NO: 59 - DNA, 22 bp, Artificial sequence
  Synthetic polynucleotide
gtaatggaacgtgtcggacact

SEQ ID NO: 60 - DNA, 22 bp, Artificial sequence
  Synthetic polynucleotide
tgcccagttgtaccatctcccc

SEQ ID NO: 61 - DNA, 22 bp, Artificial sequence
  Synthetic polynucleotide
acgggtcaacatggtagagggg

SEQ ID NO: 62 - DNA, 583 bp, Artificial sequence
  Synthetic polynucleotide
cgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggcc